(19) United States
(12) Patent Application Publication
(10) Pub. No.: US 2023/0034263 A1
(43) Pub. Date: Feb. 2, 2023

(54) COMPOSITIONS AND METHODS FOR SPATIAL PROFILING OF BIOLOGICAL MATERIALS USING TIME-RESOLVED LUMINESCENCE MEASUREMENTS

(71) Applicant: The Regents of the University of California, Oakland, CA (US)

(72) Inventors: Weian ZHAO, Irvine, CA (US); Enrico GRATTON, San Clemente, CA (US); Jan ZIMAK, Irvine, CA (US); Tam VU, San Jose, CA (US); Alexander VALLMITJANA, Irvine, CA (US); Joshua GU, Irvine, CA (US); Per Niklas HEDDE, Irvine, CA (US); Lorenzo SCIPIONI, Irvine, CA (US)

(21) Appl. No.: 17/778,302

(22) PCT Filed: Nov. 19, 2020
(86) PCT No.: PCT/US2020/061282

§ 371 Date: May 19, 2022

Related U.S. Application Data
(60) Provisional application No. 62/937,422, filed on Nov. 19, 2019.

(51) Int. Cl. C12Q 1/6841; C12Q 1/682; C12Q 1/6818

(52) U.S. Cl. CPC C12Q 1/6841; C12Q 1/682; C12Q 1/6818

(57) ABSTRACT

In alternative embodiments, provided are compositions, including products of manufacture and kits, and methods, for in situ spatial profiling of biological materials such as DNA, RNA and protein in cells, tissues, and organisms for investigating biology and for conducting biomarker/drug discovery and development, and for clinical pathology and diagnosis. In alternative embodiments, provided are compositions, including products of manufacture and kits, and methods, for spatially determining, visualizing or quantifying target biological materials comprising in situ staining of a biological sample with one or a plurality of probes that are labeled with light-emitting moieties that exhibit or are encoded with distinct luminescence lifetime (and, optionally, spectrum) characteristics; followed by time-resolved luminescence imaging, measurement and analysis.

### RELATED APPLICATIONS

This Patent Convention Treaty (PCT) International Application claims the benefit of priority to U.S. Provisional Application Ser. No. 62/937,422, filed Nov. 19, 2019. The aforementioned application is expressly incorporated herein by reference in its entirety and for all purposes.

### STATEMENT AS TO FEDERALLY SPONSORED RESEARCH

This invention was made with government support under National Institutes of Health (NIH), DHHS, grant nos. 1U54CA217378-01A1 and P41-GM103540. The government has certain rights in the invention.

### TECHNICAL FIELD

This invention generally relates to processes for in situ spatial profiling of biological materials such as DNA, RNA and protein in cells, tissues, and organisms for investigating biology and for conducting biomarker/drug discovery and development, and for clinical pathology and diagnosis. In alternative embodiments, provided are compositions, including products of manufacture and kits, and methods, for spatially determining, visualizing or quantifying target biological materials comprising in situ staining of a biological sample with one or a plurality of probes that are labeled with light-emitting moieties that exhibit or are encoded with distinct luminescence lifetime (and, optionally, spectrum) characteristics; followed by time-resolved luminescence imaging, measurement and analysis.

### BACKGROUND

A major unmet need in biology and clinical diagnosis is rapid, high resolution, and cost-effective identification, quantification and validation of molecular markers associated with genomics, epigenomics, transcriptomics, proteomics and metabolomics. In particular, tools which can spatially determine, validate and integrate all the molecular information will rapidly accelerate the pace of progress in many important fields such as cancer, immunology, tissue engineering, stem cell, developmental biology, biomarker/drug discovery and development, disease diagnosis, prognosis, companion diagnostics, patient stratification, and precision and personalized medicine.

The complete multi-omics detection and quantification of biological materials such as DNA, RNA, and protein elements in cells, tissues and organisms therefore become critical to research and clinical applications. Of particular importance is the in situ spatial analysis of these biomaterials that are key to determine their presence, numbers, locations, structural relationships, dynamics, and interactions. Such analyses, requiring advanced microscopy techniques, can provide information about gene and protein expression, cell type, cell state, cellular processes, cell-cell and cell-niche communications on the community- and tissue-scale in heterogeneous samples. Such tools will also be useful to validate data obtained from other existing technologies such as single-cell RNA sequencing.

Conventional in situ spatial detection include immunohistochemistry (for example, for protein detection) and in situ hybridization (ISH) including fluorescence in situ hybridization (FISH) for DNA or RNA analyses. However, they require intensive individual optimization. The fluorescence intensity-based measurement can only analyze a small number of target analytes due to limited spectral channels of conventional epi-fluorescence or confocal microscopes. In addition, it has been a great challenge to effectively detect these molecules in conventional immunohistochemistry and FISH due to low signal-to-noise (SNR) ratio. Recent strategies such as RNAscope® (Advanced Cell Diagnostics) can boost signal by using collections of many oligonucleotide probes via additional rounds of hybridization to adaptor sequences. However, these methods are complicated and costly and do not easily scale up nor are easily automatable.

### SUMMARY

In alternative embodiments, provided are methods for spatially determining, visualizing or quantifying target biological materials, comprising:

(a) providing a biological sample;

(b) in situ staining of the sample with one or a plurality of probes labeled with light-emitting moieties that exhibit or are encoded with distinct or defined luminescence lifetime characteristics, wherein the one or the plurality of probes specifically bind to the target biological materials,

and optionally the one or a plurality of probes also exhibit or are encoded with a distinct spectrum,

and optionally the distinct or defined luminescence lifetime characteristics or properties of the light-emitting moieties of the plurality of probes comprise or are defined by characteristics, numbers, orders, positions, patterns, configurations, orientations, and interactions modulated by distance, structural and/or architectural relations of the plurality of probes; and

(c) imaging of the biological sample using a time-resolved luminescence, and

(d) measuring the spatial profiles of the target biological materials in the biological sample.

In alternative embodiments or aspects of methods as provided herein:
the biological sample comprises cells, a tissue, a fresh frozen tissue, a formalin-fixed paraffin-embedded (FFPE) tissue, an optimum cutting temperature (OCT) preserved tissue, a biopsy or an organism;
the cells comprise mammalian cells, and optionally the mammalian cells comprise human or mouse cells, or are derived from human or mouse cells;
the target biological materials comprise an RNA, and optionally the RNA comprises an mRNA;
the target biological materials comprise a DNA, and optionally the DNA comprises a chromosomal DNA or a genomic DNA;
the target biological materials comprise a protein or a peptide, and optionally the protein or peptide comprises an epitope;
the target biological materials comprise multiple types of omics markers, wherein optionally the omics markers comprise nucleic acids and proteins, and optionally the omics markers are detected simultaneously;
the one or the plurality of probes comprise an nucleic acid probes or a plurality of nucleic acid probes, or an oligonucleotide or a plurality of pooled oligonucleotides, and optionally the nucleic acid or oligonucleotide probes have an average length of between about 6 and 300 nucleotides, or between about between about 10 and 200 nucleotides, or between about between about 20 and 100 nucleotides;
the one or the plurality of probes comprises an antibody-oligonucleotide conjugate; or, the one or the plurality of probes comprise a readout domain or domains that allow further binding of a plurality of additional probes, and optionally the readout domain or domains are generated through a target-binding mediated event, and optionally the target-binding mediated event comprises an enzymatic or a branched amplification event;
the target biological materials comprise a plurality of target molecules, and each target molecule is stained with (or is specifically bound by) 1 probe, at least about 2 probes, at least about 3 probes, at least about 4 probes, at least about 5 probes, at least about 10 probes, at least about 20 probes, at least about 30 probes, at least about 40 probes, at least about 50 probes, at least about 100 probes or more, or wherein each target molecule is stained with (or is specifically bound by) between about 2 and 100 probes;
the biological sample is stained with a plurality of same or different probes simultaneously or sequentially, or wherein the in situ staining of the biological sample comprises staining with a plurality of probes simultaneously or sequentially;
the light-emitting moieties comprise fluorophores that exhibit lifetime ranging from between about 0.2 nanoseconds to about 20 nanoseconds;
the time-resolved luminescence comprises a Fluorescence Lifetime Imaging Microscope (FLIM) comprising:
   (a) irradiating the stained sample with a modulated light source;
   (b) detecting photons emitted by the sample using a detector or a set of detectors;
   (c) measuring and analyzing a multitude of emitting species comprising use of a (spectral) phasor approach, wherein optionally the analyzing comprises use of spectra-phasor;
   (d) analyzing multiple lifetime and spectral components in single pixels using an algorithm; and
   (e) identifying and quantitating the target biological molecules at single-molecule resolution from a static or time-lapse 2D image or 3D z-stack, optionally using an image-processing component;
the multi-component analysis phasor algorithm allows unmixing multiple lifetime and spectral components in the same pixel of an image and is used to ensure fidelity of target detection and to decode a plurality of target moieties within the same diffraction-limited voxel;
the time-resolved luminescence imaging and analysis are further combined with spectral or hyperspectral imaging comprising parallel Digital Frequency Domain (DFD) electronics or camera-based system light sheet imaging with a multidimensional phasor;
the hyperspectral imaging and/or lifetime imaging system is equipped with sine/cosine filters;
one, two, three, four, five, six, seven, eight, nine, ten, 100, 1,000, or 10,000 or more different nucleic acid or protein molecules are simultaneously detected or imaged on the same sample in a multiplex fashion, wherein optionally the nucleic acid comprises an RNA or a DNA; and/or
the method further comprises placing the biological sample in a compartment that allows fluid flow for processing the sample, and optionally the compartment that allows fluid flow comprises a microfluidic system.

In alternative embodiments, provided are methods for designing combinatory, luminescence spectrum and/or lifetime encoded probes and using them to detect target molecules, comprising:
(a) providing a target molecule or a plurality of target molecules in a sample. wherein optionally the sample is a biological sample, and optionally the biological sample comprises a cell, and optionally the cell is a mammalian or a human cell;
(b) providing a plurality of probes that:
   (i) specifically bind to the target molecule(s), and
   (ii) comprise a label comprising a light-emitting moiety that exhibits a distinct luminescence lifetime characteristic, and optionally also comprising a spectrum characteristic;
(c) contacting the plurality of probes with the target molecule or the plurality of target molecules under conditions wherein the plurality of probes can specifically bind to the target molecule or the plurality of target molecules, thereby combinatorially labeling the target molecule or the plurality of target molecules; and
(d) detecting and measuring the specific binding of the plurality of probes with the target molecule or the plurality of target molecules using a time-resolved luminescence method,
wherein when measured and analyzed using the time-resolved luminescence method, each combinatorially labeled target molecule or molecules can elicit a unique luminescence lifetime (and optionally also spectrum) signature on a phasor or a spectra-phasor plot, which can identify x, y or x, y, z coordinates of the target molecule or molecules at a single-molecule resolution in the sample,
and optionally further comprising (e), a codebook or index library to decode and identify a target of interest.

In alternative embodiments, provided are methods for designing combinatory spectrum encoded probes and using them to detect target molecules, comprising:

(a) providing a target molecule or a plurality of target molecules in a sample. wherein optionally the sample is a biological sample, and optionally the biological sample comprises a cell, and optionally the cell is a mammalian or a human cell;

(b) providing a plurality of probes that:
(i) specifically bind to the target molecule(s), and
(ii) comprise a label comprising a light-emitting moiety that exhibits a distinct spectrum characteristic;

(c) contacting the plurality of probes with the target molecule or the plurality of target molecules under conditions wherein the plurality of probes can specifically bind to the target molecule or the plurality of target molecules, thereby combinatorially labeling the target molecule or the plurality of target molecules; and

(d) detecting and measuring the specific binding of the plurality of probes with the target molecule or the plurality of target molecules using a hyperspectral imaging comprising parallel Digital Frequency Domain (DFD) electronics or camera-based system light sheet imaging with a multidimensional phasor,

wherein when measured and analyzed using the spectrally resolved luminescence method, each combinatorially labeled target molecule or molecules can elicit a unique spectrum signature on a phasor plot, which can identify x, y or x, y, z coordinates of the target molecule or molecules at a single-molecule resolution in the sample,

and optionally further comprising (e), a codebook or index library to decode and identify a target of interest.

In alternative embodiments, the luminescence lifetime and/or spectrum characteristics are encoded through a combinatorial combination of light-emitting moieties' characteristics, numbers, orders, positions, patterns, configurations, orientations, and interactions modulated by distance, structural and architectural relations.

In alternative embodiments, the interactions modulated by distance, structural and architectural relations, or the interactions between light-emitting moieties, are modulated using Förster resonance energy transfer (FRET) comprising use of a FRET pair of dyes, wherein optionally the distance between the FRET pair of dyes range from 2 nm to 10 nm,

and optionally the FRET phenomena are used as an error correction mechanism at the nanometer level to resolve multiple target molecules in the same voxel.

In alternative embodiments, provided are compositions or products of manufacture comprising:

(a) a plurality of primary target molecule probes, each primary target molecule probe comprising:
(i) a biorecognition motif with a complementary region which can selectively bind to a specific portion or region of the target molecule in the sample, and
(ii) an extension element or a “read-out” or “adapter” element that can selectively bind to a specific portion or region of a secondary probe;

(b) a second plurality of secondary probes, each secondary probe comprising:
(i) a region which binds specifically to the corresponding extension element on the primary probe, and optionally further comprising a signal amplification or a signal amplification component, and
(ii) a light-emitting moiety or moieties conjugated to one or both ends of the secondary probe with each light-emitting moiety comprising a signal that is distinctly different from each other light-emitting moiety in luminescence spectrum and/or lifetime characteristic.

In alternative embodiments, provided are compositions or products of manufacture:
at least one light-emitting moiety comprises a fluorophore;
at least one of the plurality of primary target molecule probes comprises an oligonucleotide; and/or
at least one of the plurality of primary target molecule probes comprises an antibody or antibody binding fragment thereof.

In alternative embodiments, provided are kits comprising:
(a) at least one set of probes capable of binding to a target molecule or a plurality of target molecules;
(b) at least one set of probes conjugated to or that can bind to a light-emitting moiety or moieties; and
(c) at least one agent used for sample fixation, permeabilization, hybridization, blocking, washing, buffering and/or mounting,
and optionally further comprising a signal amplification or a signal amplification component, wherein optionally the signal amplification comprises tyramide signal amplification (TSA) and other peroxidase-based signal amplification or rolling circle amplification.

In alternative embodiments of kits as provided herein, the target molecule or the plurality of target molecules comprise a target biological material or a biological molecule, wherein optionally the target biological material or the biological molecule comprises a nucleic acid, and optionally the nucleic acid comprises an RNA or an mRNA, or a DNA, wherein optionally the DNA comprises a chromosomal DNA or a genomic DNA, and optionally the target biological material or the biological molecule comprises a protein or a peptide, and optionally the protein or peptide comprises an epitope. In alternative embodiments, the at least one set of probes comprise nucleic acid or oligonucleotide probes that can bind to the plurality of target molecules, or the biological materials, by specifically hybridizing to a target sequence. In alternative embodiments, the at least one set of probes comprise antibody-oligonucleotide conjugates. In alternative embodiments, the nucleic acid or oligonucleotide probes have an average length of between about 6 and 300 nucleotides. In alternative embodiments, the kits as provided herein comprise instructions for practicing methods as provided herein.

In alternative embodiments, provided are computer-implemented methods comprising: a computer-implemented method comprising a subset of, substantially all, or all of the steps as set forth in the flow chart of FIG. 21.

In alternative embodiments, provided are computer program products for processing data, the computer program product comprising: computer-executable logic contained on a computer-readable medium and configured for causing the following computer-executed steps to occur: executing a computer-implemented method as provided herein.

In alternative embodiments, provided are Graphical User Interface (GUI) computer program products comprising: program instructions for running, processing and/or implementing: (a) a computer-implemented method as provided herein; (b) a computer program product as provided herein.

In alternative embodiments, provided are computer systems comprising a processor and a data storage device wherein said data storage device has stored thereon: (a) a computer-implemented method as provided herein; (b) a computer program product as provided herein; (c) a Graphical User Interface (GUI) computer program product as provided herein; or, (d) a combination thereof.

In alternative embodiments, provided are non-transitory memory medium comprising program instructions for running, processing and/or implementing: (a) a computer-implemented method as provided herein; (b) a computer program product as provided herein; (c) a Graphical User Interface (GUI) computer program product as provided herein; (d) a computer system as provided herein; or (e) a combination thereof.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The drawings set forth herein are illustrative of exemplary embodiments provided herein and are not meant to limit the scope of the invention as encompassed by the claims.

Figures are described in detail herein.

FIG. 1A-D schematically illustrate an exemplary process of disclosed time-resolved spatial analysis:

FIG. 1A illustrates sample(s) to be labeled and imaged can be alive or fixed. The sample(s) comprise cells and target molecules to be analyzed;

FIG. 1B illustrates primary label probes are added to the sample to bind to targets of interest (for example nucleic acids, proteins);

FIG. 1C illustrates the optional step of using secondary label probes which can be added to bind to the primary labels, often through a “readout” domain;

FIG. 1D illustrates how labeled targets can be measured and imaged under a microscope that interrogates the lifetime of the labeled light-emitting moieties, often along with their other characteristics such as intensity, emission wavenumbers, etc.

FIG. 1E illustrates exemplary analysis tools such as a phasor plot can be used to analyze the lifetime and/or intensity, and the like, of the labeled targets; and

FIG. 1F illustrates exemplary labeled targets eliciting the encoded lifetime (optionally together with intensity or spectrum) signature are identified to indicate presence of targets, often in a multiplexed fashion.

FIG. 2A-H schematically illustrate exemplary general lifetime barcoding probe designs and target labeling strategies:

FIG. 2A illustrates exemplary single labeling: targets are labeled with only one type of probe, and the said probes are generally tethered with a luminophore or light-emitting moiety.

FIG. 2B illustrates exemplary dual FRET labeling: targets are labeled with a pair of different luminophores such as the Förster resonance energy transfer (FRET) fluorophore pairs or fluorophore-quencher pairs;

FIG. 2C illustrates exemplary distance-based FRET dual labeling: targets are labeled can be labeled with the same FRET pair but with varying distances to modulate interactions between the fluorophores;

FIG. 2D illustrates exemplary amplification-based labeling: targets are labeled with a moiety such as an enzyme which can react with a substrate to produce light and induce signal amplification;

FIG. 2E illustrates exemplary bioluminescence Resonance Energy Transfer (BRET)-based labeling: targets are labeled with a moiety which can react with a substrate to produce bioluminescence, and a corresponding donor moiety label will react to this induced signal for BRET to occur;

FIG. 2F illustrates exemplary branch-based labeling: targets are labeled with a series of labeling steps to create a larger branch-like structure that allows additional labels to be attached, targets can then be decorated with more labels to increase signal.

FIG. 2G illustrates exemplary combinatorial-based labeling: targets are labeled with a different combination of labels, this example barcoding strategy can help identify the target and allow high level multiplexing; and

FIG. 2H illustrates exemplary molecular Beacon-based labeling: targets are labeled with molecular beacons or hairpins that open up and fluoresce upon binding to the target.

FIG. 3 schematically illustrates an exemplary instrument or multiplexed setup that can be used to conduct the lifetime measurement and analyses as provided herein.

FIG. 4A-C illustrate an exemplary lifetime-based multiplex detection using distance-based FRET:

FIG. 4A illustrates an exemplary representative intensity-based image of a labeled sample which has been excited at the same wavelength and collected with a single detector is shown;

FIG. 4B illustrates that each pixel in a representative image may contribute to a position on the phasor plot, where, in this case, 10 different populations may be segmented, and each population may represent a different target with a unique encoding label based on molecular interactions such as FRET, BRET, combinatorial, and the like, and this barcode labeling scheme can permit enormous simultaneous multiplexing capabilities while using only a minimal number of probes; and

FIG. 4C illustrates that each target may be analyzed for its lifetime and/or intensity signature for identification, and 10 different targets can be identified in this field of view;

FIG. 5A-D illustrate an exemplary method comprising multiplexing by combinatorial labeling using fluorescence lifetime imaging:

FIG. 5A illustrates a schematic of an experiment demonstrating combinatorial labeling and multiplexed detection of mRNA transcripts (mNeon Green in this illustration) using fluorophores excitable at the same wavelength, three samples were labeled with Alexa 647 only, Atto 647 only, or both Alexa 647 and Atto 647;

FIG. 5B illustrates that the sample labeled with only Atto 647, gating the pixels of the corresponding image by the expected lifetime of Atto 647 revealed only the labeled mRNA targets while gating the pixels by any other lifetime revealed only background;

FIG. 5C illustrates that for the sample labeled with Alexa 647 only, gating the pixels by the expected lifetime of Alexa 647 revealed only the labeled mRNA targets; and

FIG. 5D illustrates that for the sample labeled with both Atto 647 and Alexa 647, gating the pixels by the expected lifetime of the linear combination (blend of fluorophore lifetimes) of Atto 647 and Alexa 647 revealed only the dual fluorophore labeled mRNA targets.

FIG. 6A-D illustrate images of detected mRNA transcripts in optimum cutting temperature (OCT) preserved mouse skin tissue using exemplary methods as provided herein, where UBC mRNA transcripts from mouse skin tissue preserved via OCT medium were processed and labeled with Alexa 647:

FIG. 6A illustrates an intensity image of a mouse skin tissue sample with transcripts labeled with Alexa 647;

FIG. 6B illustrates a Phasor plot of the pixels from both images FIG. 6A and FIG. 6C;

FIG. 6C illustrates an intensity image of a mouse skin tissue sample not stained with any primary labels targeting its UBC transcripts to serve as a negative control;

FIG. 6D illustrates that when gated for the expected lifetime of Alexa 647, only the pixels constituting the labeled UBC mRNA transcripts in FIG. 6A are highlighted; and

FIG. 6E illustrates that when gated for any other lifetime, only pixels constituting the highly fluctuating autofluorescence background are highlighted.

FIG. 7A-E illustrate images of detected mRNA transcripts in formalin-fixed paraffin-embedded (FFPE) preserved mouse colon tissue using exemplary methods as provided herein, UBC mRNA transcripts from mouse colon tissue preserved via FFPE medium were processed and labeled with Alexa 647:

FIG. 7A illustrates an intensity image of a mouse colon tissue sample with transcripts labeled with Alexa 647;

FIG. 7B illustrates a Phasor plot of the pixels from both images FIG. 7A and FIG. 7C;

FIG. 7C illustrates an intensity image of a mouse colon tissue sample not stained with any primary labels targeting its UBC transcripts to serve as a negative control;

FIG. 7D illustrates that when gated for the expected lifetime of Alexa 647, only the pixels constituting the labeled UBC mRNA transcripts in FIG. 7A are highlighted; and

FIG. 7E illustrates that when gated for any other lifetime, only pixels constituting the highly fluctuating autofluorescence background are highlighted.

FIG. 8A-D illustrate images of time-resolved detection used in combination of super-resolution imaging (stimulated emission depletion (STED) is used as an illustration) of mRNA transcripts using exemplary methods as provided herein:

FIG. 8A illustrates an image of a sample containing UBC mRNA transcripts stained with Alexa 647 is shown under regular confocal imaging;

FIG. 8B illustrates a region of interest from the same confocal image is shown; and

FIG. 8C and FIG. 8D illustrate this same region of interest but with STED imaging; increasing the depletion laser strength leads to an increase in resolution (left to right); particular points are marked where the increase in resolution allows individual structures which is a blur in the confocal image to be resolved in the STED image.

FIG. 9A-D illustrate images using an automated phasor-FLIM (Fluorescence Lifetime Imaging) target segmentation and counting software using exemplary methods as provided herein:

FIG. 9A illustrates a representative image of a sample containing three types of mRNA transcripts labeled with a different fluorophore is taken on a microscope with FLIM capabilities;

FIG. 9B illustrates inputting a representative image into an exemplary program as provided herein, as illustrated in the flow diagram of FIG. 21, allows the software to register and phasor transform each pixel photon arrival time for a position on the phasor plot;

FIG. 9C illustrates that afterward, populations of pixels with distinct lifetimes may be resolved and segmented automatically based on the chosen fluorophores used in the experiment;

FIG. 9D illustrates that each population on the phasor plot may correspond to a different gene expression target and may be processed via a different mask allowing individual puncta to be detected and identified; and

FIG. 9E illustrates that the software then can potentially remap the original image with each transcript highlighted with its corresponding unique shape or color code for target and spatial identification.

FIG. 10A-C illustrate 12-plex mRNA detection in samples of SW480 colon cancer cells tagged combinatorically with lifetime and spectrum encoded probes:

FIG. 10A illustrates the combinatorial example of 12 different genes (DCLK1, SEMA3D, LGR5, EGFR, MERTK, MAFB, NCOA3, POLR2A, MTOR, MKI67, BRCA1, and NCOA2) tagged with combinations of 6 probes (image is a z-projection of the entire stack);

FIG. 10B illustrates the resulting number of counts of each transcript after assignation; and

FIG. 10C illustrates the location of individual transcripts in 3D, scale bar is 10 μm.

FIG. 11A-I illustrate automated analysis in phasor space and detection of puncta:

FIG. 11A illustrates the intensity image of a cell labeled with fluorescent probes;

FIG. 11B illustrates the mapping of the pixels in the phasor plot with three distinct populations identified;

FIG. 11C, FIG. 11D, FIG. 11E illustrate the three lifetime populations unmixed in the phasor space can be mapped back to the original image;

FIG. 11F illustrates that image stacks can be acquired to map a sample in all 3 dimensions;

FIG. 11G illustrates a color-coded population overlay in a single plane unmixed by phasor;

FIG. 11H illustrates data showing that individual cell puncta counts can be quantified; and

FIG. 11I illustrates quantification showing puncta relative intensity, lifetime, x, y, z coordinate in the sample, decoded label, and corresponding gene; note that the panels here are for illustrative purposes and are not necessarily corresponding to each other.

FIG. 12A-D illustrate 6-plex mRNA detection with an exemplary method as provided herein:

FIG. 12A illustrates a sample containing microglia cells labeled and detected for the presence of 6 types of mRNA (TGFB, MDM2, P2RY12, LPL, MERTK, and MAFB), with each labeled with a different fluorophore; a composite intensity image of 5 spectra channels (488, 532, 565, 590, and 647 nm) indicate all but two target genes can be differentiated by their intensity wavelength;

FIG. 12B illustrates that when detecting and analyzing puncta at the 647 nm spectra, the two genes, MERTK (ATTO 647) and MAFB (ALEXA 647) could not be distinguished because both fluorophores exhibited the same spectra (red); and

FIG. 12C illustrates that when analyzed using lifetime, magenta (MAFB) and red (MERTK) can now be separated, and a phasor mapped image with its representative phasor plot (FIG. 12D) are shown.

FIG. 13A-C schematically illustrate an exemplary process of protein and mRNA codetection:

FIG. 13A schematically illustrates that sample(s) to be labeled and imaged can be alive or fixed, and the sample(s) comprise cells and target molecules including both proteins and mRNAs to be analyzed;

FIG. 13B schematically illustrates that primary label oligo probes and antibodies (or antibody-oligo conjugates) are added, optionally sequentially, to the sample to bind to targets of interest including mRNAs and proteins, respectively; and

FIG. 13C schematically illustrates that optionally, secondary label probes are added to bind to the primary labels or antibodies (or antibody-oligo conjugates), often through a “readout” domain, and while this schematic only shows the symbols of two mRNA targets and two protein targets, it should be understood that in alternative embodiments, exemplary technology as provided herein can profile two or more different target molecules in each species category simultaneously.

FIG. 14A-D schematically illustrate an exemplary process of protein labeling and detection:

FIG. 14A schematically illustrates that proteins can be directly detected using antibody-dye conjugates;

FIG. 14B, FIG. 14C, and FIG. 14D schematically illustrate that proteins are stained with antibodies that are conjugated with oligonucleotides or nucleic acid strands on which secondary dye-conjugated probes are hybridized:

FIG. 14B schematically illustrates that the same secondary dye-conjugated probes can be used;

FIG. 14C schematically illustrates that two or more different secondary dye-conjugated probes can be used to barcode a protein target in a combinatorial fashion; and,

FIG. 14D schematically illustrates an example where the oligonucleotide stand tethered on antibodies can initiate an amplification reaction (for example, rolling circle amplification (RCA)) to generate a long nucleic acid strand on which secondary dye-conjugated probes are hybridized.

FIG. 15A-H illustrate images of simultaneous 4-plex co-detection of protein and mRNA in colon cancer SW480 cells using exemplary methods as provided herein:

FIG. 15A-C: FIG. 15A illustrates images of nucleus staining DAPI, FIG. 15B illustrates images of the proteins tubulin, and FIG. 15C illustrates images of vimentin, where tubulin and vimentin were labeled with TUBB4A mouse and VIM rabbit primary antibody respectively, and secondary antibodies goat anti-mouse Alexa 488 and donkey anti-rabbit TRITC were then used respectively.

FIG. 15D-F illustrates images showing that, using this exemplary method, the two targets within the 647 nm spectral channel (FIG. 15D, both mRNA mTOR and mRNA POLR2A) were separated as seen in FIG. 15E (mRNA POLR2A) and FIG. 15F (mRNA mTOR), and mRNA targets POLR2A and mTOR labeled with target specific primary probes were hybridized, then secondary probes with Alexa 647 and Atto 647 were hybridized to the primary probes respectively;

FIG. 15G illustrates an image showing the merge of proteins (Tubulin and Vimentin) and RNA targets (POLR2A and mTOR); and

FIG. 15H graphically illustrates the Signal-to-Noise Ratio (SNR) and puncta count analysis as performed for the mRNA targets.

FIG. 16A-B illustrate detecting mRNA transcripts in highly scattering and autofluorescent tissues (image legend on the right):

FIG. 16A top image schematically illustrates how human FFPE skin sections were labeled with probes targeting POLR2A with ALEXA 647, and the two images (intensity 647 nm is the upper image and FLIM 647 nm is the lower image) show that FLIM (Fluorescence Lifetime Imaging) effectively discriminates the labeled puncta (green circles) against autofluorescent moieties (red circles) with similar Signal-to-Noise Ratio (SNR); and

FIG. 16B top image schematically illustrates how a scrambled control non-complementary towards POLR2A served as a negative control to highlight the diverse autofluorescent moieties which can be present in highly autofluorescent tissues, and the two images are: intensity 647 nm is the upper image and FLIM 647 nm is the lower image.

FIG. 17A-C illustrates combinatorial labeling of mRNA transcripts in highly scattering and autofluorescent human skin FFPE tissues improving detection efficiency and fidelity, where POLR2A was labeled with ATTO 565 (FIG. 17C) and ALEXA 647 (FIG. 17A), the puncta that appear in both the 565 nm and 647 nm channel (green circles) are classified as POLR2A puncta while autofluorescent moieties (red circles) with similar SNR are separated, and the 590 nm channel (FIG. 17B) served as a negative control to demonstrate the specificity of the POLR2A labeling only appearing in the channels it was intended to be in.

FIG. 18 illustrates exemplary 4-plex combinatorial mRNA detection in highly scattering and autofluorescent human skin FFPE tissues, where the upper images schematically illustrates the protocol used, which used a total of 4 fluorophores, BRCA1 (red circles), NCOA2 (green circles), and MKI67 (purple circles) mRNA were each labeled with 2 fluorophores and UBC was labeled with a single fluorophore (ATTO 565), the targets combinatorially labeled with puncta circled appeared in both channels it was labeled in, where the left lower image is FLIM at 647 nm, the middle lower image is FLIM at 590 nm and the right lower image is FLIM at 565 nm.

FIG. 19A-D illustrates combining lifetime measurements with FRET for fluorescence barcoding/decoding:

FIG. 19A graphically illustrates the theoretical behavior on the lifetime phasor as one reduces the distance between the FRET probes;

FIG. 19B schematically illustrates the tagging mRNA transcripts with FRET probe pairs at different distances;

FIG. 19C illustrates real images of transcripts tagged with only the donor, and the probe pair at two different distances, where the left image is no acceptor, the middle image is d (distance) at 25 base pairs (bp), and the right image is d (distance) at 12 base pairs (bp); and

FIG. 19D graphically illustrates a phasor plot that resolves the different cases of the images in FIG. 19C.

FIG. 20 schematically illustrates the workflow of an exemplary automated high-throughput probe design pipeline as provided herein, where the input sequence is screened for user defined parameters and the list of candidate probes is then aligned to the genome; unique probes and the next-generation sequencing (NGS) data for sample are aligned to the genome to filter for probes that bind to high expression regions; see Table 1, below and FIG. 22, for a final list of probes (SEQ ID NOs:1-173 generated with specifications of each probe (for example, location, sequencing read count, and alignment percentage), and as illustrated in FIG. 20 an additional list of NGS-validated probes: cgaccaagccgcttctccacagacg (SEQ ID NO:212), gaaagcgactaaacaggcaggaccc (SEQ ID NO:213), cttccatggtgacggtcgtgaaggg (SEQ ID NO:214), and cggagcaaaatatgttccaattgtgtt (SEQ ID NO: 215), where the figure also shows that some sequences were filtered out (SEQ ID NOs:174-211), see Table 2 and below for explanations as to why these sequences were removed.

FIG. 21 schematically illustrates an exemplary workflow of image processing and analysis pipeline, algorithm or software for target molecule (shown as “puncta”) detection and classification following our spectral/FLIM imaging.

FIG. 22 illustrates Table 1, which shows: mTOR NGS (next generation sequencing) Aligned Result, a table of NGS validated mTOR probes generated by the exemplary BLAT_Aligner script which removes probes that are nonspecific using BLAT and aligns the NGS data with probes for this gene to obtain the read count for each probe region, where each probe includes the following information: number of base pairs that align, sequenceID, probe size, chromosome number, chromosome size, chromosome start position, chromosome end position, probe start, sequence, match percentage, read count average from NGS dataset 1, and read count average from NGS dataset 2 (if available).

The patent or application file contains at least one drawing executed in color. Copies of this patent or patent application publication with color drawing(s) will be provided by the Office upon request and payment of the necessary fee.

Like reference symbols in the various drawings indicate like elements.

### DETAILED DESCRIPTION

In alternative embodiments, provided are compositions, including products of manufacture and kits, and methods, for in situ spatial profiling of biological materials such as DNA, RNA and protein in cells, tissues, and organisms for investigating biology, conducting biomarker/drug discovery and development, and for clinical pathology and diagnosis.

In alternative embodiments, provided are methods for designing luminescence lifetime encoded probes for in situ spatial profiling of biological materials by using time-resolved luminescence techniques. In one aspect, methods are provided for spatial profiling of biological materials in cells, tissues, and organisms comprising: a) a sample, b) in situ staining or binding of the target analysts with one or a plurality of probes that are labeled with light-emitting moieties that exhibit or are encoded with distinct or defined luminescence lifetime characteristics, and c) subsequent time-resolved imaging, measurement and analysis to determine, visualize and quantify the said biological materials. In alternative embodiments, the said spatial profiling methodology, when performed at different timepoints of a biological process or disease progression, can collect additional temporal information of the biological system as well.

In another aspect, the said sample can be cells, tissues, spheroids, neurospheres, organoids, 3-dimensional (3-D) cell culture, tumoroids, and organisms that can be from any species. In some aspects, these samples are live or living. In yet other aspects, the samples are fixed and preserved. In some aspects, the sample can be a biopsy. In some aspects, the samples are formalin-fixed, paraffin-embedded (FFPE). In another embodiment, the samples are optimum cutting temperature (OCT) preserved tissue. And another embodiment, the samples are fresh frozen tissue.

In some aspects, the target molecules or molecular processes are or involve deoxyribonucleic acid (DNA). In another aspect, the target molecules or molecular processes are or involve ribonucleic acid (RNA) such as messenger RNAs or mRNAs. In some aspects, the target molecules or molecular processes are or involve proteins or (poly)peptides. In yet other aspects, the target molecules or molecular processes are or involve any other types of cellular constituents or externally administered moieties including, but not limited to, lipids, carbohydrates, small molecules, biologics, and pharmaceuticals. In yet another aspect, the target molecules or molecular processes are or involve pathogenic materials such as DNA, RNA or protein from a bacterium, a virus, a fungus, a parasite, and a pathogen.

In some aspects, probes as provided herein, when analyzed through luminescence lifetime imaging, can spatially detect or report the presence and dynamics of biological molecule(s) or biological process(es). Often, the probes collectively possess at least two functions: a) target binding, and b) light-emitting. Often, upon staining, each given target molecule can carry one probe molecule or a plurality of same or different probe molecules. In alternative aspects, the said probes are oligonucleotides. In some aspects, the oligonucleotides are modified. In alternative aspects, the said oligonucleotide probes comprise domains or target sequences that specifically hybridize with the target nucleic acids. In some aspects, the said oligonucleotide probes comprise readout or read sequences which additional probes can bind to. In alternative aspects, the said probes comprise at least two domains with one binding to the target molecules and the other serving as a readout to further binding to additional probes. In alternative aspects, a set of additional (for example secondary, tertiary, etc.) probes are added to the sample which generally bind to their corresponding readout elements on the primary probes or target binding-mediated (amplification) products. In some aspects, the staining process may involve sequential binding of additional probes or multiple around of binding and unbinding steps. In alternative aspects, the said probes comprise moieties for specific target biorecognition including, but not limited to, antibodies or antigen binding fragments thereof (for example, Fab fragments or single-domain antibodies (sdAb), also known as nanobodies) and their derivatives, nucleic acid or peptide aptamers, carbohydrates, proteins, CRISPR-associated (Cas) proteins, or synthetic binders. In alternative aspects, the said probes comprise moieties for additional functions including, for example, biotin-(strept)avidin for conjugation, or horseradish peroxidase (HRP) for signal amplification.

In alternative embodiments, at least one set of the said probes are labeled with, conjugated to, or complexed with light-emitting moieties including, for example dyes, fluorophores, chromophore(s), phosphorescent element(s), bioluminescent element(s), or inorganic materials that exhibit one or a plurality of lifetime characteristics. In alternative aspects, one target molecule is labeled with one or multiple light-emitting moieties at a time. In alternative aspects, the light-emitting moieties are complexed with the target molecules indirectly through “adaptor” molecules such as a nucleic acid sequence, hapten, secondary antibody or an engineered, orthogonal tag.

In alternative embodiments, provided are methods and concepts to design and use luminescence lifetime encoded (or barcoding) probes. In alternative aspects, the said luminescence is fluorescence and the said light-emitting moieties are fluorophores.

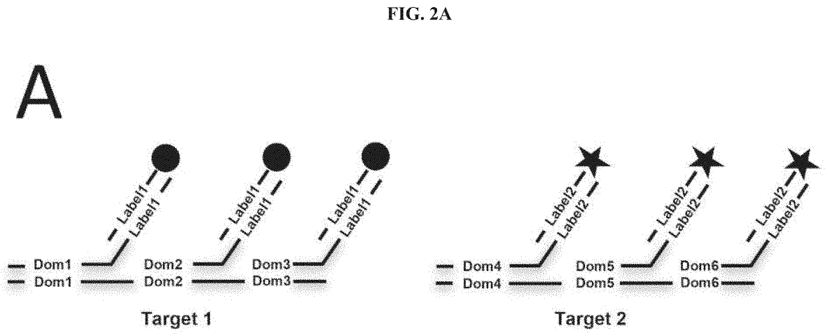

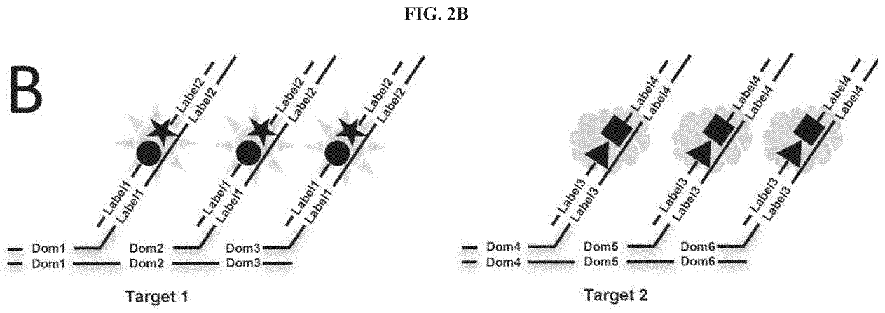

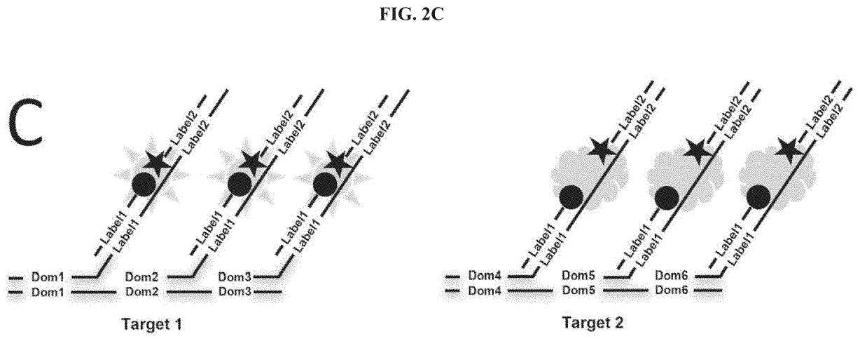

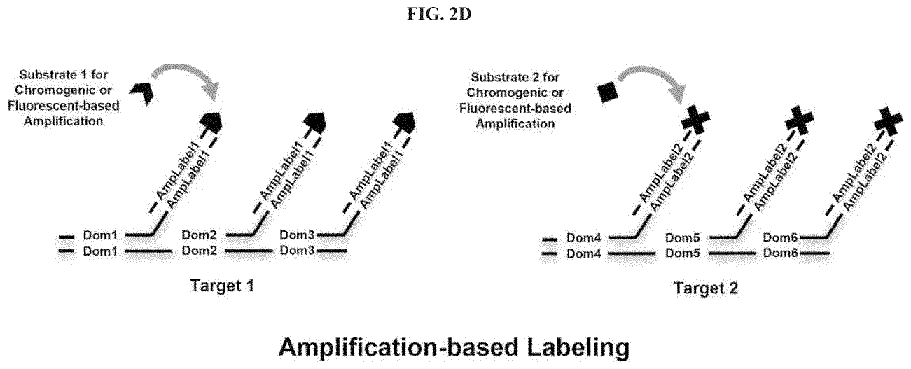

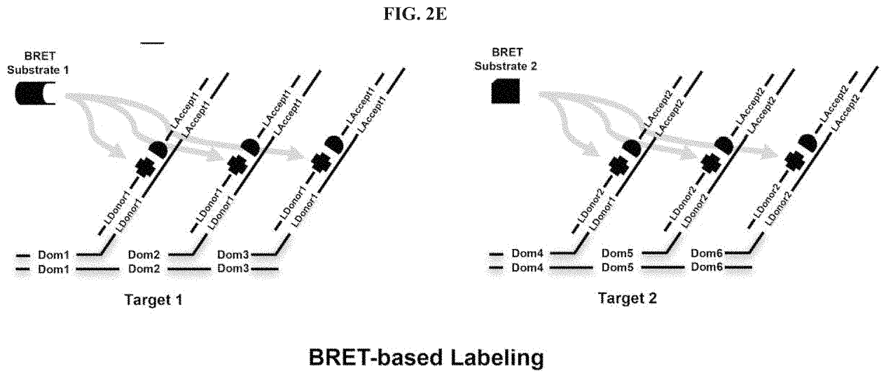

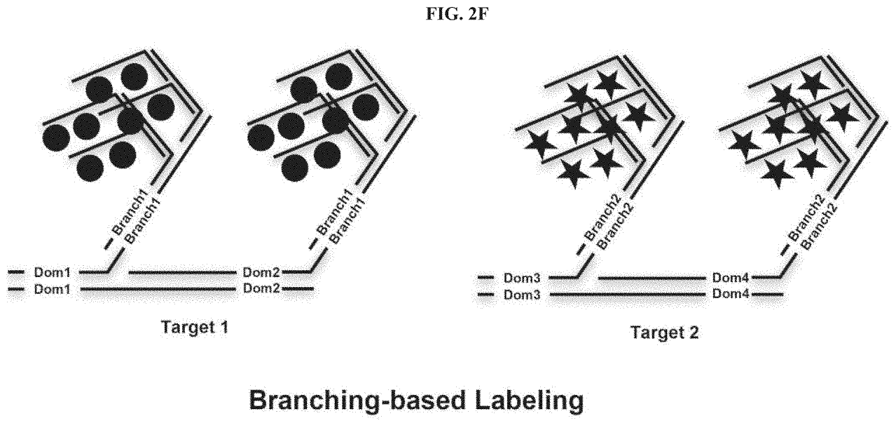

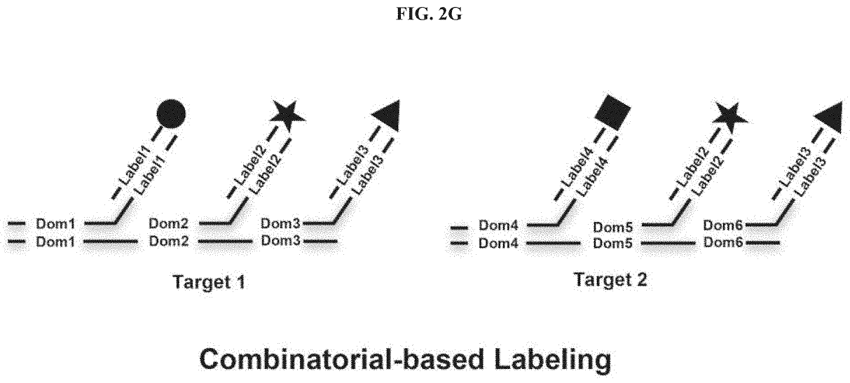

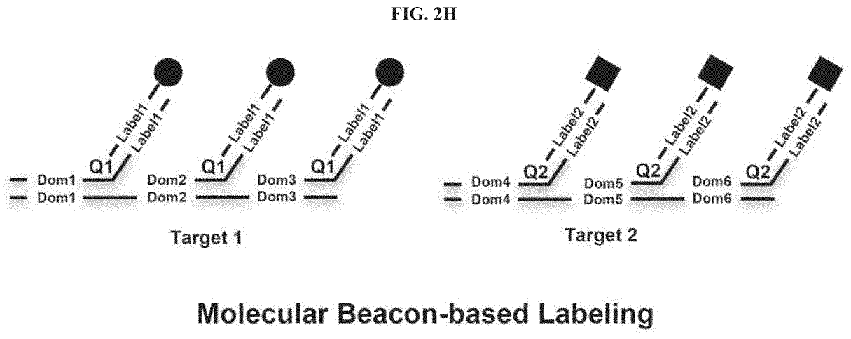

In alternative embodiments provided are various choices of fluorophores, molecular configurations, orientations or interactions of fluorophore-labeled probes that combinatorially encode or barcode distinct lifetime signatures. Some of these designs are illustrated in FIG. 2. In alternative aspects, the said lifetime ranges from about 0.2 nanoseconds to about 20 nanoseconds. In alternative aspects, the target molecule is labeled with one type of probes whereas in other cases, it is labeled with two or more different probes, optionally, with same or different luminescence lifetime signatures. In alternative aspects, the probes are labeled with one fluorophore on each probe. In some other cases, the probes are labeled with two or more fluorophores on each probe. In alternative aspects, the fluorophores are designed to interact with each other using mechanisms such as Förster resonance energy transfer (FRET) and (de)quenching to modulate lifetime. In another embodiment, targets are labeled with probes that comprise one, dual, or multiple FRET pairs that can generate a FRET response upon residing in close proximity to each other. Each FRET pair has a different molecular configuration which can elicit a different detectable lifetime signature, which permits high degree multiplexing capabilities. In further embodiments, different probe orientations (for example head to tail, head to head, etc.) can be adapted when assembled on the target or readout domains to further modulate the luminescence lifetime properties of the light-emitting moieties. In alternative aspects, a spatial pattern of labeled spots (spatial barcoding) to yield a unique optical signature (for example lifetime, lifetime/spectral combination) for each target in spatial analysis.

In alternative embodiments, provided are kits for detecting one or multiple target biological materials. In some aspects, the said kit comprises a series of primary, secondary, tertiary etc. probes, either used singly or in combinations, for target detection, signal reading and amplification, multiplexing, or barcoding purposes. The kit may also comprise various other components such as agents for sample fixation, permeabilization, hybridization, blocking, washing, buffering, mounting, etc.

In some embodiments, the sample(s) stained with probes are imaged or analyzed using a microscope that is equipped for lifetime measurement and analysis. In some aspects, the said time-resolved analyses with encoded lifetime probes are used in combination of existing techniques such as intensity and spectral analysis, superresolution imaging and multiphoton imaging to enhance performance on target identification, resolution, quantitation, SNR, speed, and/or tissue depth. In some aspects, the said time-resolved techniques or methods include, but not limited to, Fluorescence Lifetime Imaging Microscope (FLIM), FRET-FLIM, Fluorescence Lifetime Cross-Correlation Spectroscopy, Phosphorescence Lifetime Imaging Microscope (PLIM), (Bio)luminescence Lifetime Imaging Microscope (BLIM), and their related variations.

In alternative embodiments, a FLIM system used for the methods disclosed herein, which can comprise: a) a modulated light source that irradiates the stained sample, b) a detector or a set of detectors for detecting photons emitted by the sample, and, c) a phasor approach to analyze lifetime data and decode the lifetime information encoded in the probe design to detect, quantify and spatially visualize the target molecules in the sample, and optionally, d) a spectral phasor approach to measure and analyze the multitude of emitting species separated on the basis of their emission spectrum.

In some aspects, a major enabling technical advance to embodiments as provided herein is the use of the phasor method to rapidly and precisely measure fluorescence and phosphorescence lifetimes in samples as described in this application. The phasor method derives from the digital frequency domain hardware and software that permits using all the photons detected from a sample with a simple and inexpensive hardware for example FLIMbox and the representation of the decay data using polar coordinates. This method allows the precise measurement of many lifetime components simultaneously without performing fits of the decay data, making automatic detection of a plurality of molecular species in the same field of view possible as shown in some of the figures of this application.

In some embodiments, another major enabling capability is the “spectral phasor” analysis from the same samples with respect to both lifetime and spectrum characteristics. For instance, recent technologies, for example based on the Sin-Cos filter approach, allow rapid and precise hyperspectral measurements in the same microscope (or optical device) and the same sample. This capability is important for the methods described in this application because it allows determination of both lifetime and spectrum simultaneously, increasing the combinatorial of probes that can be employed in the sample.

In another embodiment, the disclosed methods are further enabled by multi-component analysis in the same pixel. This technical feature allows determination of multiple lifetime and spectral components in the same pixel of a sample. The method is based on the law of linear combination of components valid after transformation of the decay curves to phasors. In principle, the linear combination rule is valid for an arbitrary number of components. This technical advance allows us to rapidly examine a large area of the sample at low resolution to determine which molecular species are present in this area and then, if the components of interest are present, we can zoom in in the sample to determine the exact spatial location of these components.

Furthermore, the high resolution for multiple component analysis in one pixel allows further decoding or resolving of encoded lifetime (optionally and spectral) information to effectively detect and quantify target molecules.

We point out that the phasor approach to lifetime and spectral component and the resolution of multiple species in a large area of the sample are new technologies not used before for the purposes described in this application. These techniques are applicable to transparent as well as highly scattering sample such as deep tissues. Equally important is that these techniques allow rapid and unsupervised analysis of large samples and that the techniques are amenable to artificial intelligence that can further improve quantification.

Some capabilities, features, specifications or advantages of the disclosed embodiments for spatial profiling of biological materials compared to existing technologies include, but not limited to, a) improving multiplex capability (for example can detect one molecule or 10s, 100s, 1,000s, or 10,000s target molecules simultaneously) by adding the time dimension to the conventional intensity-spectra based measurements. This is particularly useful to profile multiple targets or the whole transcriptome or proteome in a cell, b) reducing sample background or autofluorescence and therefore improving detection sensitivity, SNR, and efficiency. In alternative aspects, sample and tissue autofluorescence can be used in conjunction with external probes to effectively identify and quantify various target molecules or biological processes, c) capable of spatially (for example molecular location, distribution) determine and quantify biomaterials and their dynamics in a three dimensional (3D) fashion, d) high resolution to visualize single cells, subcellular features or single molecules, e) broad dynamic range from one molecule to 10s, 100s, 1,000s, and to 10,000s molecules per cell, f) highly robust, accurate and quantitative, g) high throughput, or can analyze a large number of samples quickly), h) high generalizability, or the disclosed methods can be used to detect any biological targets. Minimal optimization is needed to design from one target to another. In fact, probe design can be streamlined using computational tools, and i) low cost.

These exemplary disclosed embodiments can have broad utilities and applications in areas including, but not limited to, research, biology (for example synthetic biology), immunology, immunotherapy, biomarker/drug discovery and development, pathology, disease screening, diagnosis, prognosis, companion diagnostics, precision medicine, cell engineering, cancer, neurological disorders, infectious diseases, neuroscience, brain and neurological disorders, development and stem cell biology, diabetes, metabolic disorders, autoimmune disorders, and inflammation.

In alternative embodiments, high throughput and high-plex spatial profiling technology as provided herein can broadly enable scientists and clinicians to better study cancer biology and to develop precision diagnostics and treatments for cancer. Cancer biologists have started to realize, only recently, how heterogeneous gene (and protein) expression is and how many different cell identities/states there are in tumors. In other words, the dynamic cell fate is defined spatiotemporally by the expression of multiple (rather than a single) genes. Therefore, to fully characterize cells in situ we need to be able to assess multiple transcripts (and proteins) within the same cell, which can be readily addressed by using methods as provided herein, which use through direct, highly multiplexing, in situ biomarker profiling in a single round of staining and imaging.

Several exemplary applications requiring high-plex in situ analysis that are broadly representative in both basic cancer biology and clinical companion diagnostics (CDx) for stratified care, including for example:
1) Examining within-cell correlations and location in gene expression sampled among heterogeneous cells will inform gene regulatory mechanisms, which we cannot get from bulk measurements.
2) Single-cell RNA sequencing (scRNAseq) returns cell identities in the form of rather long “differentially expressed gene lists” that “define” cell types. However, the clustering process is subjective, variable and error-prone. The only way to validate whether a pattern of gene expression really defines a cell type, or conflates multiple cell types, is through multiplex spatial transcriptomics.
3) Patient derived materials are often available in limiting quantity and generating hundreds of sections to test for many markers is tedious and non-feasible.

Multiplexing is the only efficient way of doing this. In particular, in cancer diagnosis, prognosis, and patient stratification for combination therapy, especially in immunotherapy, physicians would now want to analyze tumor biopsies for a large number of markers. In alternative embodiments, provided are methods able to analyze liquid biopsies and suspension cells that are coated onto a substrate such as peripheral blood mononuclear cell (PBMC), circulating tumor cells (CTCs) and cytospin slides of bone marrow aspirates. In alternative embodiments, multiplexing biomarker analysis of CTCs using technology as provided herein can find applications for basic research, cancer detection, surveillance and recurrence monitoring and drug response evaluation.

Isolation and preparation of suspension cells including, but not limited to, CTCs on a substrate for imaging purposes are established in the art. For example, briefly, a typical CTC preparation workflow can comprise a) a patient peripheral blood sample (for example, 7.5 ml) is collected via venipuncture into an appropriate collection tube which optionally contains fixative to stabilize the blood sample; b) the blood tube can be shipped at room temperature; c) the samples can be processed to separate CTCs using e.g. gradient centrifugation, immunomagnetic cell separation, or a microfluidic device; d) separated CTC cells can be resuspended and spread onto positive-charged glass slides as monolayer. The slides can be analyzed immediately or air dried and stored in −80° C. for long period of time. The prepared CTC sample can then be stained and analyzed using spatialomic technology as provided herein.

While we focus much on research, medical and clinical applications, it should be understood that the disclosed methods are not limited to these applications. For example, methods as provided herein can find various utilities in agriculture and environmental applications, and only a few examples are mentioned herein. The disclosed embodiments can be used to investigate the following biological molecules, events, dynamics or processes including for example cell metabolism, cell state/status (for example division, proliferation, differentiation), molecular interactions (for example protein-protein interactions, protein-nucleic acid interactions, receptor-ligand binding), transcription, translation, modification, cellular environment, biomolecule and bioparticle mobility or rigidity, trafficking, movement, cell migration, chromosome dynamics, nuclear structures, biomolecule activity, conformation, orientation, alignment, nuclear organization, gene expression and activation in a temporal and spatial fashion, transcript abundance, predicting or identification of target expressing cell type, transcription, mRNA alternate initiation, splicing, translation, post-translational modifications, structural, conformational changes, molecular folding or any other biological functions. Furthermore, the disclosed embodiments can find many applications in the clinic, including for example genetic testing, detection of single nucleotide polymorphisms (SNPs), detection of disease-associated aberrations, chromosome defects, chromosomal aberrations, copy number quantification, cancer diagnosis, tumor detection, biomarker assay development, companion diagnostics to screen and strategize patients for treatment (for example profiling immune checkpoint inhibitors such as PD-1 and PDL-1 in tumor tissue). The disclosed embodiments can also serve as in vivo diagnostics, based on in situ staining of synthetic probes inside of an organism (for example human) using, for example, a fiber optic. Furthermore, the system can be fully automated and/or operate with a multi-well plate (for example 96 or 384 well plates) or other high through sample systems. The system can be made portable for point-of-care or on-site applications. Additionally, the tools as provided herein can complement or validate data obtained from other existing technologies that are often incapable of spatial analysis for example gene expression obtained using single cell RNA sequencing.

Provided herein are compositions and methodologies to label and image biological materials or molecules within or on cells, tissues, organs, or organisms. In some embodiments, the genomic, epigenomic, proteomic, metabolomic elements of the sample are labeled and detected on a live sample. In alternative embodiments, a live sample can be a sample harboring inside or on top of a microfluidics device, substrate such as tissue culture treated plastic, or the live sample can be naturally residing in the organism. In other embodiments, these elements may be labeled and detected on a sample that has been preserved or fixed with reagents such as paraformaldehyde, acetone, and formalin.

Examples of exemplary approaches as provided herein are described; however, these only recapitulate alternative embodiments as provided herein and do not limit the various aspects embodiments as provided herein can take or comprise of. A more detailed description of each exemplary embodiment is further described in the following sections.

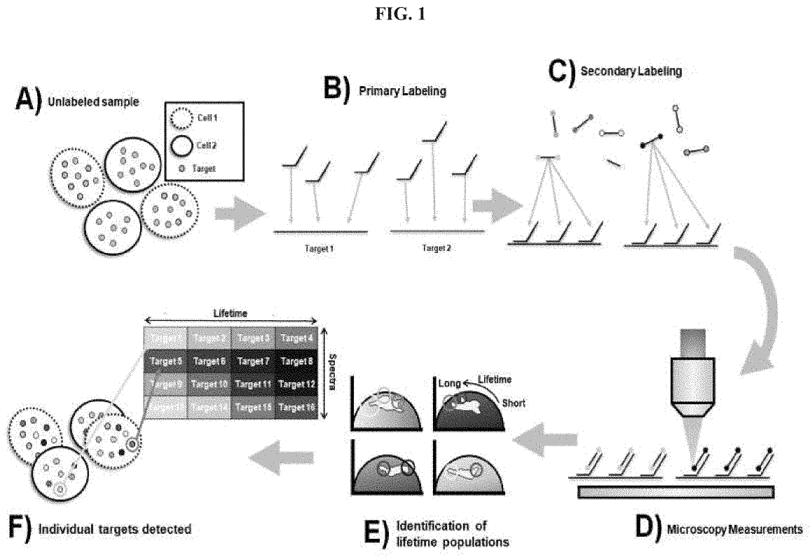

FIG. 1 depicts an exemplary method as provided herein. In Step 0, a sample is portrayed as a cluster of cells which can be alive or fixed. In alternative aspects, this sample can be of mammalian origin and in the form of tissues, organoids, organs, or even a complete organism. In other cases, this sample can be any component with viral, bacterial, archaeal, or eukarya origin. The following Step 1 depicts the addition of primary probes which can adhere to the complementary ligand or target of interest. Primary probes may be comprised of a biorecognition motif including, but not limited to, nucleic acids, modified nucleic acids, proteins, antibodies or antigen binding fragments thereof (for example, Fab fragments or single-domain antibodies (sdAb), also known as nanobodies), enzymes, carbohydrates, aptamers, peptides, lipids, biotin, engineered tag or any combination of these molecules and their modified counterparts that can bind to the specific target. Generally, these probes have a complementary region which can selectively bind to a specific portion or region of the target molecule or substrate. The primary probes should bind to only one target of interest but may also bind to multiple same or different target molecules or target epitopes. For example, the same oligonucleotide probe can bind to multiple areas of a chromosome if there is homology between similar targets of interest, for example satellite sequences in multiple areas of a chromosome. In alternative aspects, the primary probes are labeled with light-emitting moieties such as fluorophores. In yet other cases, the primary probes may contain an extension element (sometimes referred to as “read-out” or “adapter” element) which can be coupled to additional downstream labeling steps to conjugate light-emitting moieties such as fluorophores. This extension element like the primary probe itself can be comprised of for example, nucleic acids, modified nucleic acids, proteins, antibodies or antigen binding fragments thereof (for example, Fab fragments or single-domain antibodies (sdAb), also known as nanobodies), enzymes, carbohydrates, aptamers, peptides, lipids, biotin, engineered tag or any combination of these molecules and their modified counterparts. For some instances, the primary probes, upon binding to the target analyte, can trigger a downstream amplification step to generate molecular products on which additional probes can be labeled.

In Step 2, for some cases, a set of additional (for example secondary, tertiary, etc.) probes are added to the sample which generally bind to their corresponding extension elements on the primary probes or target binding-mediated (amplification) products. A secondary probe may bind to only one primary probe but may also bind to multiple primary probes if more complex binding is required (for example branching in FIG. 2F). In alternative aspects, a secondary probe may bind to a different target without the corresponding extension elements. In the exemplary embodiment shown in FIG. 1, only two labeling steps are shown. However, in other embodiments, multiple labeling steps may be used and can be of any number. Similarly, a set of tertiary probes may bind to already bound secondary probes, and so on. These additional (for example secondary, tertiary, etc.) probes can be comprised of for example nucleic acids, modified nucleic acids, proteins, antibodies or antigen binding fragments thereof (for example, Fab fragments or single-domain antibodies (sdAb), also known as nanobodies), enzymes, carbohydrates, aptamers, peptides, lipids, biotin, engineered tag, or any combination of these molecules and their modified counterparts. In this exemplary embodiment, the secondary probes are double conjugated to fluorophores on each end. In other embodiments, the secondary probes can be triple conjugated or conjugated to any number of fluorophores. In alternative aspects, these additional probes are labeled with light-emitting moieties other than fluorophores including, but not limited to, chromophore(s), phosphorescent element(s), bioluminescent element(s), or inorganic materials such as quantum dots that exhibit distinct lifetime characteristics. In yet other embodiments, several different light-emitting moieties are assembled on the said primary and/or additional probes for combinatorial barcoding lifetime and/or spectrum, which can be used to detect multiple different target analytes in a high degree multiplex assay. It should also be understood that for all the probes as provided herein, they can be modified or conjugated using standard chemical or enzymatic methods with moieties to introduce additional functionality (for example antibodies or antigen binding fragments thereof (for example, Fab fragments or single-domain antibodies (sdAb), also known as nanobodies), hapten, biotin, etc.).

After labeling, the decorated targets are then imaged under a microscope (Step 3). This microscope can be a commercial microscope or a custom-built microscope. This microscope can take the form of any configuration or setup such as a portable stand-alone instrument, phone application, supplemental gadget to a phone, or a benchtop appliance. In this particular embodiment, the technique used to image these labeled targets as illustrated in Step 3 is FLIM. In other embodiments, this technique can be polarization-based, STED, structured illumination, confocal microscopy, etc. Additionally, this technique can be any integrated combination of these imaging modalities. Further details on the potential embodiments this technique can take will be described in greater detail in the following sections.

Step 4 depicts one of the possible methods that can be used to analyze the labeled targets of interests. Depicted here is a phasor plot approach to identify molecules based on fluorescence lifetime and spectral behavior. However, any transformation of data involving replotting pixels of an image into a new subspace for further analysis may be used. Furthermore, shown in the phasor plot are different populations of pixels that can be separated by lifetime and/or spectrum. Each population can represent a different type of target of interest on the sample. In some aspects, molecules with combinatorially labeled fluorophores that elicit a unique lifetime signature or phasor position on the plot may represent one of these populations of pixels. In other aspects, targets labeled with molecules with particular FRET pairs that elicit a different unique lifetime signature may represent one of these populations. Essentially, any molecular interactions which can create a distinct detectable signature may represent a unique population of pixels that is distinguishable from each other in this manner. These molecular interactions may be intensity-based, lifetime-based, or chromogenic-based.

Step 5 shows the usage of a codebook to identify the detected target. Since there is an enormous variety of potentially different molecular interactions that may exist and be detected in the exemplary labeling approach as illustrated in FIG. 2, a codebook that pairs a certain molecular interaction to a certain marker of interest may be used to encode a significant number of potential biomarkers for post analytical identification.

In another aspect, the said sample can be (cultured) cells, tissues, spheroids, neurospheres, organoids, 3-dimensional (3-D) cell culture, tumoroids, engineered (human) organs, embryoid bodies or organisms that are either live (or living) or fixed and preserved. In alternative embodiments the sample comprises both cellular and extracellular materials. In some aspects, the sample is a biopsy (for example tumor, colon, or bone marrow sample) or a blood sample for clinical pathology and disease diagnosis purposes. In alternative aspects, the sample is from a subject, for example, an animal, mammal, plant, fungi, archaebacteria, eubacteria, or protist. In alternative aspects, the sample is derived from for example a human, mouse, rat, monkey, or pig or naturally residing in the organism. In other cases, the sample can be an entire organism such as zebrafish and Drosophila. In alternative aspects, the samples are tumor tissues. In cases of fixed samples, the samples can be fixed or preserved using standard methods including both physical (for example cryo-preservation (freeze drying), heating, micro-waving) and chemical means using various fixatives such as formalin, (para)formaldehyde, acetone, osmium tetroxide, methanol, and ethanol. In some aspects, the samples are formalin-fixed, paraffin-embedded (FFPE). Fixation can preserve the structure and components of a biological sample for durable, stable, long term storage in a variety of different conditions. In alternative embodiments, the samples can be fresh frozen tissue, or the samples can be optimum cutting temperature (OCT) preserved tissue.

In alternative embodiments, an advantage of methods as provided herein is that the time-resolved measurements can reduce or remove sample background or autofluorescence and therefore improve detection sensitivity, SNR, and efficiency. For instance, subtraction of a background signal can be done through for example multi-harmonic Fourier transform spectroscopy and frequency-domain analysis. In alternative embodiments, sample and tissue autofluorescence can be used in conjunction with external probes to effectively identify and quantify various target molecules or biological processes.

In some aspects, the said sample can be mounted on a substrate that is plain glass, glass rendered electrostatic via physical or chemical treatment, glass chemically conjugated to adhesive ligands, tissue culture-treated plastic, polydimethylsiloxane (PDMS), polypropylene, or any type of material which can allow biological materials to adhere to. In alternative aspects, the samples are fixed in a matrix material such as (hydro)gels or polymers (for example agarose and polyacrylamide). In another aspect, the sample can be expanded and further processed (for example capture, conjugation, digestion, washing) to facilitate probe binding and improve imaging quality, such as those used in expansion microscopy and Clear Lipid-exchanged Anatomically Rigid Imaging/immunostaining-compatible Tissue hyYdrogel (CLARITY) (see for example Chung et al. Nature 2013, vol 497:332-337; Du et al. Exp Ther Med. 2018 September vol 16(3):1567-1576.). In some aspects, the cells that constitute the said sample can include for example a primary cell, cancer cell, tumor cell, immune cell (for example T cell, B cell, NK cell, macrophage, monocyte, neutrophil, dendritic cell, mast cell), neural cells, engineered cell, fused cell, hybridoma, therapeutic cell, stem cell, (induced) pluripotent stem cell, progenitor cell, adult cell (for example fibroblast), eukaryotic cell, prokaryotic cell, animal cell, plant cell, bacterial cell, yeast cell, fungal cell, archaeal cell, eubacterial or a mixture of the aforementioned cell types.

In one embodiment, the said sample to be analyzed is placed in a simple flow compartment on top of a microscope where staining occurs before imaging. In another embodiment, the flow compartment can comprise a network of fluidic and/or microfluidic channels for additional sample processing steps (for example, capture, conjugation, digestion, washing) in addition to sample staining. In yet another embodiment, a higher sample throughput flow compartment can be used to analyze a plurality of samples. In this embodiment, the flow compartment comprises a plurality of fluidic and/or microfluidic networks generally known as a microfluidic system.

In some aspects, the target biological materials or molecular processes comprise or involve one or a plurality of biological molecules that exist within, on or outside of a cell. Often, one or multiple different targets are analyzed in a singleplex or multiplex fashion. In alternative aspects, the said biological molecules are nucleic acids, polynucleotides, oligonucleotides, DNA, chromosomal DNA, genomic DNA (gDNA), introns, mitochondrial DNA, complementary DNA (cDNA), plasmid DNA, RNA, coding RNAs, mRNAs, tRNAs, snRNAs, shRNAs, guide RNAs, rRNAs, poly(A)RNAs, transcripts (such as nascent transcripts), non-coding RNAs, regulatory RNAs, microRNAs, siRNA, mature RNAs, nascent RNAs, circular RNAs (circRNAs), competitive endogenous RNAs (ceRNAs) and nuclear pre-mRNAs. In some aspects, the target nucleic acids can be endogenous or exogenously introduced including, but not limited to, viral DNA or RNA, recombinant DNA or RNA, bacterial DNA or RNA, and other pathogenic DNA or RNA. In alternative aspects, the said nucleic acid targets can exist in nuclear, cytoplasmic or extracellular space. In some aspects, the said biological molecules are endogenous or exogenously introduced proteins, peptides and polypeptides, and their derivatives. In yet other aspects, the target molecules or molecular processes are or involve any other types of cellular constituents or externally administered moieties including, but not limited to, lipids, carbohydrates, small molecules, biologics, and pharmaceuticals. In some aspects, the said biological molecules are modified derivatives through endogenous or exogenous processes including chemical or enzymatic reactions such as synthetic, chemically modified nucleic acids, epigenetic modifications of DNA, post-transcriptional RNA modifications, and post-translational protein modifications. In some aspects, the said biological molecules are molecular complexes comprising any of the above-mentioned moieties as a subunit. In some aspects, the said biological molecules are cell membrane (or cell wall) or transmembrane constituents (for example GPCRs). In another aspect, the said biological molecules exist in the extracellular environment such as secreted factors or extracellular matrix constituents (for example, collagen). Often, the said biological molecules are signaling molecules, receptors, growth factors, or transcriptional targets of major signaling pathways.

Probes as provided herein, when analyzed through lifetime (and optionally together with spectral) imaging or spectral imaging, can spatially detect or report the presence and dynamics of biological molecule(s) or biological process(es). At least one set of probes as provided herein are therefore labeled, conjugated or complexed with light-emitting moieties. Together, one or multiple sets of probes should possess at least two functions: a) target binding, and b) light-emitting. Depending the target biological molecule(s) or biological process(es), at least one set of probes may be comprised of a biorecognition or an affinity motif including, but not limited to, nucleic acids, modified nucleic acids, receptors, proteins, antibodies or antigen binding fragments thereof (for example, Fab fragments or single-domain antibodies (sdAb), also known as nanobodies), enzymes, carbohydrates, aptamers, peptides, lipids, biotin, engineered tag or any combination of these molecules and their modified counterparts that can bind to the specific target. In alternative aspects, at least one set of probes are covalently or non-covalently bound to light-emitting moieties.

In alternative embodiments, provided are methods and uses of nucleic acid probes for fluorescence in situ hybridization (FISH) applications as one exemplary demonstration. In this case, the target molecule is a DNA or RNA. The probes are oligonucleotides, their modified counterparts, nucleic acids with modified bases, chimeric nucleic acids, peptide nucleic acids (PNA), locked nucleic acids (LNA), molecular beacons, hairpin structures, aptamers, siRNA, shRNA, or nucleic acid origami.

People skilled in the art can use various tools including both computational and manual methods available to design such in situ hybridization probes (see, for example, Femino, A. M., et al, 1998. Visualization of single RNA transcripts in situ. Science, 280(5363), pp. 585-590; Lyubimova, A. et al, 2013. Single-molecule mRNA detection and counting in mammalian tissue. Nature protocols, 8(9), p. 1743; Tsanov, N., et al, 2016. smiFISH and FISH-quant—a flexible single RNA detection approach with super-resolution capability. Nucleic acids research, 44(22), pp.e165-e165; Raj, A. et al, 2010. Detection of individual endogenous RNA transcripts in situ using multiple singly labeled probes. In Methods in enzymology (Vol. 472, pp. 365-386). Academic Press; Yilmaz, et al, 2011. mathFISH, a web tool that uses thermodynamics-based mathematical models for in silico evaluation of oligonucleotide probes for fluorescence in situ hybridization. Applied and environmental microbiology, 77(3), 1118-1122; Rouillard, et al. 2003. OligoArray 2.0: design of oligonucleotide probes for DNA microarrays using a thermodynamic approach. Nucleic acids research, 31(12), pp. 3057-3062). In one aspect, a set of primary probes are involved and designed to specifically hybridize with the target nucleic acid sequence (for example chromosomal DNA, mRNA). In another aspect, the said primary probes also comprise “readout” or “adaptor” sequence(s) that allow further hybridization with additional (for example secondary) probes. In an exemplary example of mRNA detection, the computational tools mentioned above can initially screen specific sequence lengths for distinct GC content and maximize the numbers of primary probes per target mRNA transcript. The ‘collective’ binding of primary oligo probes to the target mRNA molecule results in the appearance of a single bright fluorescent spot. Additional tools can further interrogate probes for potential binding to other genomic targets. To this end, tools such as mathFISH, OligoArray and OligoMiner have recently been developed. For example, OligoArray can adjust probe length based on a specific narrow melting temperature range (Tₘ), the uniqueness of each probe is then verified against a BLAST database, including those for whole genome and transcriptome. The computation can be performed using the thermodynamic parameters contained in the MFOLD package. Meanwhile, Oligominer can improve the speed and flexibility of probe design by employing a Python scripting tool that utilized Bowtie2 sequence alignment tool thus reducing alignment time from days to minutes. By also employing batch processing OligoMiner can further enable the multiplexed bioinformatic design of RNA FISH probes at genome-scale. In alternative embodiments, primary oligonucleotide probes used in embodiments as provided herein can comprise 6 to 120 unmodified or modified nucleotides (nt), optionally, 20 to 30 nt in length. In other cases, the primary oligonucleotide probes can comprise at least or about 4, 5, 6, 7, 8, 9, 10, 12, 15, 18, 20, 21, 22, 23, 24, 25, 26, 27, 28, 29, 30, 33, 35, 38, 40, 43, 45, 48, or 50 nt. In alternative aspects, for a given target mRNA, a typical primary probe library consists of 1 to 120 different oligonucleotides, optionally, 20 to 60 different oligonucleotides. In other cases, a primary probe library to stain a given target mRNA can comprise at least or about 1, 2, 3, 4, 5, 6, 7, 8, 9, 10, 12, 15, 18, 20, 23, 26, 27, 28, 29, 30, 31, 32, 33, 34, 35, 36, 37, 38, 39, 40, 41, 42, 43, 44, 45, 46, 47, 48, 49, 50, 52, 54, 56, 58, or 60 different oligonucleotides. Therefore, a primary probe library can comprise any number of oligonucleotides in the range of 10s, 100s, 1000s, or 10,000s in order to profile multiple or a large number of different mRNA transcripts. In additional embodiments, the “readout” or “adaptor” sequence(s) on the primary probes are products of a target-binding triggered event or amplification reaction such as rolling circle amplification (RCA). In this case, the length of readout sequence can range from 100 nt to 1,000 nt, or greater.

In alternative embodiments, this application comprises designing probes using an automated software and retrieves the expression level of the probe binding region from sequencing data to filter for probes that bind to high expression regions. In alternative embodiments, the mRNA or coding sequence file is used as the input file and a list of probes are generated within the sequence using the user define parameters (length, GC %, melting temperature, spacing, prohibited sequences, etc.). The list of probes is then aligned to the genome to determine if the sequence is unique and specific to the target region. Unique candidate probes are then aligned to next-generation sequencing data to obtain the read count for each binding region. Probes with high read count, thus higher expression, are then placed into a final list.

In some embodiments, additional (for example secondary) probes are used to translate, sometimes together along with amplifications, multiple primary probes from a single target into a distinct barcoded lifetime signal. In the case of RNA FISH as an example, the secondary probe sequences may have identical lengths, similar melting temperature and GC content so that their hybridization properties are similar under the same conditions. The kinetic and equilibrium properties need to be similar so that oligonucleotide labelling reactions need to reach steady state at the same rate to ensure uniform transcript labelling. As false positive signal mostly comes from secondary sequences binding to off-target binding sites, sequences of additional probes need to be screen for homology to the host genome. In addition, the readout sequences need to be orthogonal to each other, meaning that they should have minimal homology between themselves to prevent binding to the wrong sequence being read out. In some embodiments, the secondary probe sequences are composed of a three-base nucleotide base composition, which minimizes secondary structure that can impede on-target binding and increase to off-target binding. Libraries and databases of over 200,000 orthogonal sequences are available online (Xu, Q., et al 2009. Design of 240,000 orthogonal 25mer DNA barcode probes. Proceedings of the National Academy of Sciences, 106(7), pp. 2289-2294). Furthermore, improved algorithms, such as the one reported in Casini, A., et al, 2014. R2oDNA designer: computational design of biologically neutral synthetic DNA sequences. ACS synthetic biology, 3(8), pp. 525-528) can auto-generate hundreds of sequences of defined length, nucleotide base composition, with specified exclusion criteria such as certain base repeats that must be excluded (for example G quadruplex). These tools as well as the ones that can screen secondary structures (for example NUPACK) are freely available and their algorithms and code are well documented online. In alternative embodiments, these additional (for example secondary) oligonucleotide probes used in embodiments as provided herein can comprise 4 to 1,000 unmodified or modified nucleotides (nt), optionally, 15 to 30 nt in length. In other cases, the additional (for example secondary) oligonucleotide probes can comprise at least or about 4, 5, 6, 7, 8, 9, 10, 11, 12, 13, 14, 15, 16, 17, 18, 19, 20, 21, 22, 23, 24, 25, 26, 27, 28, 29, 30, 33, 35, 38, 40, 43, 45, 48, 50, 60, 80, 100, 120, 160, 200, 300, 500, 1,000 nt, or greater. In additional embodiments, the “readout” or “adaptor” sequence(s) on these additional probes are products of a target-binding triggered event or amplification reaction such as branching reactions or RCA (further details are provided below). In this case, the length of readout sequence can be from 100 nt to 1,000 nt, or greater. In further embodiments, the probe GC content can range from 30 percent to 80 percent. In other exemplary embodiments, GC content can range from 35 to 65 percent, or from 40 to 70 percent. In general, oligonucleotide or polynucleotide probe sequences may not contain repeat nucleotides of more than 3 bases (for example a G quadruplex, or a C quadruplex, or a A quadruplex, or a T quadruplex). Normally, nucleotide base composition in the oligonucleotide probe has the four canonical bases (A,C,G,T). In certain embodiments it is required to have a base composition of only three bases to limit secondary structure and off-target binding. For example a base composition of (A,C,T) or (A,G,T) can be used. In other cases, non-canonical bases or modified nucleotides are used such as those found in PNA, LNA, xeno-nucleic acids (XNA) (Chaput et al, 2019. Angewandte Chemie International Edition, 58, 11570-11572).

In some embodiments, provided are methods and concepts to design and use luminescence lifetime (optionally together with spectrum) encoded or barcoded probes including fluorescence lifetime (optionally together with spectrum) encoded or barcoded probes. In alternative embodiments, methods and compositions as provided herein are used in the context of FISH for nucleic acid detection as examples, but it should be understood that they can be generally applied to any other probe designs as provided herein, and can be for detection of any other targets (for example protein imaging).

FIG. 2 depicts exemplary molecular configurations, orientations or interactions of probes which may be used to label targets of interest with. It is important to note that this figure represents only generalized classes of labelling approaches which may be used to label targets of interest but does not limit other embodiments or approaches which a target may be labelled with. In some embodiments, targets may be labeled in a way where only a single type of probe (tethered with a luminophore such as a fluorophore) is used for downstream detection and analysis (FIG. 2A). In this particular instance, a commercial fluorophore such as an Alexa 647 dye can be used to label target 1 and another commercial fluorophore such as Atto 647 can be used to label target 2. Each target will elicit a different signal upon detection which can be used to discriminate and identify the targets. However, this label is not limited to just fluorophores but can be any component which can elicit a signal or be conjugated to another counterpart or many other counterparts which can elicit a signal for example, nucleic acids, modified nucleic acids, proteins, antibodies or antigen binding fragments thereof (for example, Fab fragments or single-domain antibodies (sdAb), also known as nanobodies), enzymes, carbohydrates, aptamers, peptides, lipids, biotin, engineered tag or any combination of these molecules and their modified counterparts.

In another embodiment, targets are labeled with a FRET pair that can generate a FRET response or a fluorophore-quencher pair upon residing in close proximity to each other (FIG. 2B). Some possible FRET pairs that may be used to label targets are Cy3 and Cy5, GFP and mCherry, or CFP-YFP. Likewise, these FRET pairs may be any component or consist of any components which can induce a FRET response. Since, each FRET pair has a different molecular configuration which can elicit a different detectable signature, they can be distinguished from each other upon analysis to permit high degree multiplexing capabilities. In another embodiment, targets may be labeled with FRET pairs that vary in distance to create unique molecular configurations which can be distinguishable from each other upon detection and analysis (FIG. 2C). One target may be labeled with a FRET pair such as Cy3 and Cy5 which are 2 nm from each other while another target may be labeled with the same FRET pair which are 3 nm from each other. The distances which can be varied with the FRET distance can be anywhere from 1 nm to 12 nm, or optionally from 2 nm to 10 nm. In further embodiments, different probe orientations (for example head to tail, head to head, etc.) can be adapted when assembled on the target or readout domains to further modulate the optical properties (for example lifetime) of the light-emitting moieties on the probes. In further embodiments, dual, multiple or tandem FRET can be used to further encode lifetime (optionally together with spectrum), improve specificity and lower false positives.

In alternative aspects, targets are labeled with an amplifiable probe component which can induce reactions to deposit detectable reactive molecules (for example, enzymes that can mediate catalytic conversion of a substrate to produce light, secondary antibody-dye conjugates) to improve signal or detection of a particular target (FIG. 2D). Example components which can be used include for example enzymes such as horseradish peroxidase (HRP), alkaline phosphatase (AP), glucose oxidase (GO), β-galactosidase (BGAL), etc. Detectable reactive molecules which may be used for this labeling approach may be for example 3,3′-diaminobenzidine (DAB), Aminoethyl carbazole (AEC), Fast Red, Nitro blue tetrazolium chloride (NBT), 5-bromo-4-chloro-3 indolyl phosphate (BCIP), or 5-bromo-4-chloro-3-indolyl-β-D galactopyranoside (BCIG or X-Gal).

Further embodiments to label targets may be probes that comprise bioluminescence resonance energy transfer (BRET) pairs to create a unique detectable molecular signature or label which does not require excitation (FIG. 2E). For each BRET pair, a donor and an acceptor may react in close proximity to facilitate non-radiative energy transfer to elicit a unique detectable signal. BRET donors may be enzyme variants such as RLuc, Aequorin, Firefly or luciferase. BRET acceptors may be for example GFP, YFP, Topaz, RFP, or any other fluorophore. To facilitate this BRET response, substrates such as coelenterazine, coelenterazine h, coelenterazine deepblueC, or d-luciferin may be used.

In alternative aspects, targets are labeled sequentially with a series of probes to create a branch-like structure that clusters out from each target (FIG. 2F). Each branch cluster may be comprised of any component which can elicit a signal or any component which can be used to extend out the branch for subsequent attachment to another component that can elicit a signal. Shown in FIG. 2F is generalized example of a target which can be a nucleic acid target which may be labeled with a series of subsequent nucleic acid probes to create a nucleic acid branch entity which can then be labeled with components that can elicit a signal such as commercial fluorophores.

However, any of the components which may comprise the branch may be nucleic acids, modified nucleic acids, proteins, antibodies or antigen binding fragments thereof (for example, Fab fragments or single-domain antibodies (sdAb), also known as nanobodies), enzymes, carbohydrates, aptamers, peptides, lipids, biotin, engineered tag or any combination of these molecules and their modified counterparts. Because each branch cluster may be labeled with many unique combinations of components, there is a wide variety of potentially different molecular signatures which can be used to permit greater multiplexing capabilities for target detection. For other aspects, targets may be labeled or barcoded combinatorially (FIG. 2G). In this generalized example, different targets may be labeled with similar components but as long as they differ in one component, a different molecular signature can be detected with a microscope or any instrument that can differentiate them from each other. Target 1 may be labeled with the encoding components: circle, star, and triangle while target 2 may be labeled with the encoding components: circle, square, and triangle (circle, star, and triangle refer to different luminophores or fluorophores). Upon measurement and analysis, both detected targets may be differentiated by their unique star or square label. Since labeling targets this way scales up multiplexed detection of targets combinatorially, a small increase in the number of possible labels to use or choose out from will entail a significantly large increase in multiplexing capabilities. For instance, for the given combinatorial equation, C(n,r)=n!/[(n−r)!r!], where C is the number of combinations possible, n is the number of components that can are available, r is the number of components which is used to label each target, and ! is the factorial function, if 16 components are available to be used and only 3 components will be used in any combination to label each target, there are 560 unique possible combinations or molecular signatures which a target can be labeled with. This is a drastic increase compared to the approach shown in FIG. 2A where labeling each target with a single fluorophore allows detection up to only 16 plex.

In alternative aspects, a target can be labeled with a combination of three components while in another aspect a target may be labeled with a combination of four components which would yield an additional 1,820 unique molecular signatures to the 560 unique possible combinations to encode additional different targets of interests. In alternative embodiments, targets are labeled with 1, 2, 3, 4, 5, 6 or 7 or more components and combinations of components with no limitation. Using biophotonics techniques which may be spectral imaging or lifetime or others, each target may be labeled with a large pool of available components which can elicit a signal. Each labeling scheme can thus be encoded to represent a particular target of interest. To decode and identify the target of interest, a codebook or index library can be utilized which will be described in more detail shortly. Furthermore, these components or probes can be commercially or manually synthesized and conjugated fluorophores such as Alexa dyes which can differ by for example 5 nm emission wavenumber for example Alexa 555 vs. Alexa 560 which can be separated and distinguished by spectral imaging and spectral phasor analysis. These components can also be commercially or manually synthesized conjugated fluorophores which can be excited at the same wavelength but have different lifetime properties such as Alexa 647 which has a lifetime of 1 ns in PBS and Atto 647 which has a lifetime of 4 ns in PBS. Furthermore, these components can also have the same intensity-based and/or lifetime-based property in a particular solvent (or media) such as PBS but differ in their intensity-based and/or lifetime-based property in a different solvent. For example, Alexa 647 may have a lifetime of 1 ns in PBS but may have a lifetime of 1.65 ns in glycerol solution. If a particular component differs from another particular component in its intensity-based and/or lifetime-based property in a particular solvent but has the same properties in all other solvents, these two particular components can still be differentiated and can be used as different components for this combinatorial labeling strategy. Therefore, we can encode probe lifetime in a broad range from about 1 picosecond to about 1 second, optionally, for fluorescence lifetime from about 100 picoseconds to about 1,000 nanoseconds, and optionally, for phosphorescence lifetime lifetimes on the order of microseconds, milliseconds or longer. Furthermore, the generalized branching-based labeling method in FIG. 2F may employ the same combinatorial labelling method exemplified in FIG. 2G. As such, it is important to note that although each labeling method may look specific and unique, they can be employed in any combination with each other to permit greater multiplexing capabilities. In alternative aspects, one light-emitting moiety with known or defined lifetime can be used as a reference to calibrate or determine the lifetime of other light-emitting moieties.

In another set of embodiments, molecular beacon-based labeling may be used to label a target (FIG. 2H). Molecular beacons may be made of for example nucleic acid molecules which have a hairpin configuration in its closed state when not bound to a target and may have an elongated configuration in its opened state when bound to a target. However, molecular beacons may also be made of for example any nucleic acids, modified nucleic acids, proteins, aptamers, peptides, or any combination of these molecules and their modified counterparts that can react in this particular way. Shown in FIG. 2H is a schematic of two molecular beacons being used, one for each target. Generally only when bound to the target a molecular beacon would elicit a signal because its donor moiety is now separated from its quencher moiety to decrease quenching for a more noticeable signal. If the molecular beacon is non-specifically bound to any other non-targets, its closed configuration will elicit zero or minimal signal to facilitate greater signal to noise detection. Molecular beacons may be constructed of any size and may have donors made of commercial fluorophores or donors of any component which can elicit a signal. Acceptors may be commercial quenchers such as TAMRA, DABYCL, blackhole quencher 1 (BHQ-1), blackhole 2 (BHQ-2), or any quencher which reacts with the donor in this particular fashion.

In further aspects, as shown in explementary FIG. 2, an exemplary embodiment representing an important class of labeling methods which can be used to label targets is sequential or serial labeling, stripping, and imaging of targets to allow greater multiplexed detection by time rather than space. For example, a target may be labeled with the approach illustrated in FIG. 2A and then imaged to attain a characteristic molecular signature and detection. This label may then be removed via any reagent or physical means such as heat that can displace the component away from the target to permit subsequent labeling to that same target with another label. Another set of labels can then be re-added to the sample which can target the same target but with a different component or different targets with a similar component. Each round of labeling, imaging, and stripping can then be used to improve multiplexing capabilities. Each round can also use a different labeling approach each time to vary the possibilities. For example, target 1 may be labeled with the single labeling approaches as shown in FIG. 2A and then labeled with a combinatorial labeling approach as shown in FIG. 2G. Through this approach, an unlimited number of multiplexing capabilities is possible as long as there is enough rounds or series of labeling, imaging, and stripping that will be utilized.

In alternative embodiments, any molecular interactions which can create a distinct detectable signature may represent a unique label. These molecular interactions may be intensity-based, lifetime-based, spectral-based, chromogenic-based, or any biophotonics-based property (for example blinking). To accommodate such a diverse set of labeling possibilities for each target, a codebook may be used as a legend or an index that pairs a certain molecular interaction to a certain marker of interest to encode a significant number of potential biomarkers for post analytical identification, quantification, and spatial validation. Each measured and distinct detectable signature or unique label can thus be decoded for identification. In some embodiments, this codebook may be a simple library that matches each specific molecular label or signature to a specific target of interest on a one to one basis. In other embodiments, this codebook may be a library which encodes each target of interest with multiple labels with redundancy or degeneracy. For instance, if there are 64 labels or set of labels available to be used to label targets, even if each label or set may correspond only to a specific target, a target may correspond to multiple labels or sets. Furthermore, the codebook may encode a particular target with only one unique label but encode several different targets with the same label. For instance, a certain gene such as UBC may be labeled with Alexa 647 and Atto 647 while a family of single nucleotide polymorphism (SNP) genes such as KRAS may all be labeled the same with Atto 647 and Atto 565.

In alternative embodiments, the codebook may also employ an error correction statistical mechanism where even if a target should be labeled with 5 labels, even if two of those labels do not show up, it can still assign a probability factor to that target as being correctly identified as the particular target of interest. Another error correction mechanism which the codebook can apply can be sequential labeling error correction where if certain labels show up at certain rounds but not others, there is a likelihood that it's still that particular target of interest with a probability factor associated to it. Furthermore, the codebook may employ a certain code for a particular target for certain conditions such as being immersed in PBS where a certain intensity and/or lifetime signature may be detected while employing a different code for that same target that will elicit completely different intensity and/or lifetime signature upon changing the solvent. If certain expected labels show up in a certain solvent but does not show up in a different solvent for that same target, the probability of that target being correctly identified may be increased with a certain probability factor. In addition, the codebook may employ an error correcting cleavage mechanism where if certain specific labels which are cleavable are reimaged, a probability factor can be associated with that target to determine if the identification of that target is accurate. This codebook may also employ several biophotonic techniques simultaneously to determine if a particular target is correctly identified. Targets which are encoded to elicit certain lifetime signatures can also be detected of their polarization-based signatures to determine if that lifetime signature correctly corresponds to the particular target with a probability factor based on how well it correlates with the polarization measurement.

In alternative embodiments, this codebook can employ any combination, derivative, or sequence of the aforementioned types of coding schemes to permit greater multiplexing or more robust detection via various error correction schemes. Furthermore, this codebook may also encode certain autofluorescence signatures of naturally occurring biological or chemical components in the sample to reveal their identities. For example, fibronectin has a characteristic lifetime or polarization-based signature. This codebook may permit identification of not just components used to label targets but essentially any autofluorescence source already present in any sample as well. Indeed, the combination of lifetime barcoding of exogenous “probes” and endogenous, intrinsic lifetime signals from the sample constituents can allow increased multiplexing ability to interrogate biological molecules, processes, status or local environment (for example polarity, pH, temperature, ion concentrations, etc). For instance, using this concept, mRNA expression (detected by exogenous probes) and cellular metabolism (detected by autofluorescence via for example NAD/NADH, flavin adenine dinucleotide, and tryptophan) can be measured simultaneously (for example through morphology and artificial intelligence methods).

In some aspects, polynucleotide or oligonucleotide probes or labels described above can be synthesized through standard solid-phase synthesis and can be custom or standard ordered from any of a variety of commercial sources, such as Integrated DNA Technologies, Sigma, Thermo Scientific, Qiagen and many others. The conventional approach is limited in scale as each probe is synthesized individually. To get around this logistical constriction, multiple probes (up to about 10,000 to about 100,000 individual probe strands) can be synthesized in parallel on an array or chip. For instance, an array of probes can be synthesized on a spatially addressable solid support (for example membrane, silicon chip, plate or slide). Similarly to conventional synthesis probes are bound to the support by a cleavable linker at a unique location. (for example covalently or electrostatically). Methods of manufacturing such probes arrays (e.g microarrays) are well known in the art (Baldi et al. 2002. DNA Microarrays and Gene Expression: From Experiments to Data Analysis and Modeling, Cambridge University Press; Beaucage, 2001. Strategies in the preparation of DNA oligonucleotide arrays for diagnostic applications, Curr Med Chem 8:1213-1244; Schena, ed. 2000. Microarray Biochip Technology, pp. 19-38, Eaton Publishing; technical note “Agilent SurePrint Technology: Content centered microarray design enabling speed and flexibility” available on the web at chem.agilent.com/temp/rad01539/00039489.pdf; and references therein). Oligo arrays can be synthesized on commercially available instruments such as GMS 417 Arrayer (Affymetrix, Santa Clara, Calif.). Alternatively, pools of oligos in an array format with up to about 300 base pairs each are commercially available from Custom Array (http://www.customarrayinc.com), Twist Bioscience (www.twistbioscience.com) and IDT (www.idt.com). Those skilled in the art can further use enzymatic amplification protocols to generate probes in high quantity sufficient for spatial profiling experiments using the above-mentioned array-derived oligos as templates. An example protocol involves four steps: a) limited-cycle amplification using PCR (amplify template DNA), b) in vitro transcription (amplify templated DNA into RNA), c) reverse transcription (RT) (turn amplified RNA into cDNA), and d) degrade template RNA via alkaline hydrolysis). The RNA intermediate is utilized to maximize the quantity of nucleic acid created. These reactions although optimized at the lab bench can be scaled to commercial quantities and use commonly available enzymes (Moffitt, et al. 2016, High-throughput single-cell gene-expression profiling with multiplexed error-robust fluorescence in situ hybridization. Proceedings of the National Academy of Sciences 113.39 (2016): 11046-11051; Shah, et al, 2017. seqFISH accurately detects transcripts in single cells and reveals robust spatial organization in the hippocampus. Neuron, 94(4), pp. 752-758).

As discussed above, the probe sequences are often designed with sequences or repeats of additional binding sites for sequential hybridization to amplify the signal. Therefore, probes are often synthesized with repetitive barcode sequences or concatemers. However, when single strands beyond 200 nt are synthesized, significant numbers of synthesis errors occur. An alternative is to repetitively extend a priming strand enzymatically. This can be achieved using several methods such as primer change reaction (Femino, et al, 1998. Visualization of single RNA transcripts in situ. Science, 280(5363), pp. 585-590.), primer exchange reaction (Kishi, et al 2018. SABER enables highly multiplexed and amplified detection of DNA and RNA in cells and tissues. bioRxiv, p. 401810), programmed in situ growth of concatemers by RCA, padlock probe amplification, hybridization chain reaction (HCR), PCR, or RT-PCR. People skilled in the art can also amplify the signal through assembly of DNA structures using serial rounds of chemical ligation or sequential hybridization, programmable nucleic acid assembly in the case of an origami, or branched amplification or reaction such as those used in RNAscope® technology. In these cases, some sets of oligonucleotide probes may optionally be termed as pre-amplifiers, readout domains, amplifiers, and detectors. In some examples, each oligonucleotide probe may contain two or more pre-amplifier, readout, amplifier, and detector domains or sequences for barcoding and multiplexing purposes. These are detected by successive rounds of hybridization. In alternative aspects, oligonucleotide probes contain for example (RT)PCR primer binding domains for amplification purposes, and T7 promoter region for in vitro transcription.

In alternative embodiments, a label, modifier, functional group, biotin, dye, fluorophore or other moiety can optionally be introduced to the probe either during or after synthesis through a chemical or enzymatic process. These modifications can introduce functions such as possessing diverse abilities such as nuclease resistance, photoactivation, self-avoidance, binding to higher-ordered structures, and multicoloring. As such chemistries are generally established in the art, we only summarize several commonly used ones here using DNA probe as an example. However, it should be understood that other chemistries and moieties can also be incorporated into any probes as used in embodiments provided herein, including also protein-based as disclosed herein. For example, a biotin phosphoramidite can be incorporated during chemical or enzymatic synthesis of a polynucleotide. Alternatively, a nucleic acid molecule can be biotinylated using techniques known in the art; suitable reagents are commercially available, for example, from Pierce Biotechnology. Similarly, a nucleic acid molecule can be fluorescently labeled, for example, by using commercially available kits such as those from IDT, TriLink, Molecular Probes, Inc. or Pierce Biotechnology or by incorporating a fluorescently labeled phosphoramidite during chemical or enzymatic synthesis of a polynucleotide. Similarly, people skilled in the art can readily synthesize modified nucleic acids such as PNA, XNA, and LNA or incorporate additional functional moieties including, but not limited to, a dye, antibody, secondary antibody, antibody fragment, protein, enzyme (for example HRP), biotin, (strept)avidin, peptide, aptamer, hapten (for example, Dinitrophenyl (DNP), digoxygenin, trinitrophenyl (TNP)), pyrene, 2′-O-methyl group, engineered tag on which additional probes can bind, bead, or a nanoparticle (for example quantum dot, gold nanoparticle). Numerous standard conjugation techniques can be found in for example, Greg T. Hermanson's “Bioconjugate Techniques”, Academic Press, Third edition, 2013. In some aspects, the light-emitting moieties comprise luminophores, dyes, fluorophore(s), chromophore(s), chromogenic substrates (often used together with enzymes; for example, 3,3′,5,5′-Tetramethylbenzidine, 3,3′-Diaminobenzidine, 2,2′-azino-bis(3-ethylbenzothiazoline-6-sulphonic acid with HRP), phosphorescent materials, chemiluminescent enzymes and element(s) (for example 1,2-Bis[4-(azidomethyl)phenyl]-1,2-diphenylethene, luminol; optionally used with enzymes to generate light), bioluminescent element(s) (for example luciferase/luciferin families and their derivatives), inorganic materials such as quantum dots, or any luminescent materials including for example lanthanides and their complexes, and other metal-ligand complexes. These light-emitting moieties exhibit one or a plurality of distinct luminescence lifetime. Luminescence lifetime is generally related to the time or how quickly a luminescence decays. For example, fluorescence lifetime is the time a fluorophore spends in the excited state before returning to the ground state by emitting a photon (Weber, G. et al. 1966. Fluorescence and Phosphorescence Analysis. Principles and Applications, Interscience Publishers (J. Wiley & Sons), New York, pp. 217-240). The said lifetime is an intrinsic characteristic of the molecule and can be affected by the surrounding environment. Therefore, luminescence lifetime measurement represents a powerful tool in biology to study for example, protein-protein interactions, biomolecular mobility, biomembrane fluidity and rigidity, conformational or structural information of cellular and biomolecular constituents, chemical reaction, ion flux, cell metabolism. Depending on the light-emitting moieties, their lifetimes can range from picoseconds to hundreds of nanoseconds, or to micro-seconds, to milliseconds, or to seconds.

In some embodiments, of methods as provided herein the said light-emitting moieties are fluorophore(s) can be excited by an external light source to emit light. For example, fluorophore(s) are generally used in FLIM experiments described herein. In alternative aspects, these fluorophore(s) can be synthesized. In additional cases, these fluorophore(s) can be readily available from commercial sources and can be conjugated or complexed with the probes. In alternative embodiments, fluorophore(s) as used in methods as provided herein include, but not limited to, the BODIPY series (BODIPY 493/503, BODIPY FL-X, BODIPY FL, BODIPY R6G, BODIPY 530/550, BODIPY TMR-X, BODIPY 558/568, BODIPY 564/570, BODIPY 576/589, BODIPY 581/591, BODIPY TR-X, BODIPY 630/650-X, BODIPY 650/665-X), the Alexa series (Alexa 350, Alexa 405, Alexa 488, Alexa 514, Alexa 532, Alexa 555, ATTO 550, Alexa 568, Alexa 594, Alexa 647, Alexa 680, Alexa 750), the ATTO series (ATTO 425, ATTO 430LS, ATTO 488, ATTO 495, ATTO 514, ATTO 520, ATTO Rho6G, ATTO 542, ATTO 565, ATTO Rho3B, ATTO 490LS, ATTO Rho11, ATTO Rho12, ATTO Thio12, ATTO Rho101, ATTO 590, ATTO 610, ATTO 620, ATTO Rho14, ATTO 633, ATTO 643, ATTO 647N, ATTO 665, ATTO 655, ATTO 680, ATTO 700, ATTO 725, ATTO 740), FAM, FITC, Cy3, Cy5, PE, Coumarin, PerCP, TRITC, Texas Red, APC, quantum dots, or a fluorescent protein (for example Green Fluorescent Protein (GFP), Cyan Fluorescent Protein (CFP), and Red Fluorescent Protein (RFP)). These fluorophores can have various excitation and emission wavelengths that cover a broad range of spectrum from UV to infrared regions or with an emission wavelength from about 350 nm to about 900 nm. In alternative aspects, these light-emitting molecules comprise “split” domains (for example split fluorescent proteins, split luciferases) that, when combined, can emit light. In some embodiments, the fluorophores can serve as “quencher” to other fluorophores. Additional fluorescence quenchers can also include for example Deep Dark Quenchers (for example DDQ-I, II), Dabcyl, Eclipse quenchers, Iowa Black FQ, RQ, BHQ-1, 2, 3, QSY-7, 21, or gold nanoparticle. Fluorophore/quencher or fluorescence donor and acceptor pairs are often used in FRET and molecular beacon designs. In general, parameters including for example molar absorptivity, extinction coefficient, photo-stability, quenching, lifetime separation are considered to identify FRET or fluorophore-quencher pairs for FLIM experiments. Those skilled in the art can also employ (photo)activatable, (photo)switchable, or (photo)cleavable probes to modulate dye properties using means of external stimulus (for example heat, light) or changing local environment (for example pH, temperature). In addition, methods such as photobleaching can also be used to alter dye properties or reduce autofluorescence.

In some embodiments, the fluorophore(s) are covalently incorporated in the probes. In alternative aspects, at least one set of light-emitting moieties can be covalently incorporated into the target molecules by, for example, nick translation or recombinantly expressed (for example fluorescent proteins). In addition, each probe can contain more than one same or different fluorophores. In some aspects, light-emitting molecule-carrying probes can be subsequently conjugated to the target molecule through moieties (for example biotin) that are covalently incorporated into the target molecules. In some aspects, the probes are bound to the products resulting from amplification of the target molecule through enzymatic or non-enzymatic reactions. In other aspects, the fluorophore(s) are noncovalently complexed with the probes. For example, nucleic acid intercalating or binding dyes such as DAPI, 3,5-difluoro-4-hydroxybenzylidene imidazolinone (DFHBI), thiazole orange, Propidium Iodide, SYTO 9, and SYTOX or their its derivatives and variants can fluoresce upon nucleic acid hybridization. In some other cases, the fluorophore(s) or other light-emitting moieties can be complexed with the probes and/or with the target molecules through interactions for example pyrene moieties, forced intercalation (FIT), engineered orthogonal tags (for example peptide, RNA sequence), or through “adaptor” molecules such as a nucleic acid binding protein such as a Pumilio Homology Domain, a phage, a fluorescent protein fused protein, a scFv-antibody conjugate, an aptamer, a Cas protein, a deactivated or nuclease-deficient Cas protein (dCas) fused with a fluorescent protein, a (d)Cas/guide RNA complex, a hapten-antibody complex, a phage coat protein-fluorescent protein fusion. Those skilled in the art can also modify the target biomolecule or probe with a chemical moiety with which a second chemistry (for example click chemistry) can be conducted to tether the light-emitting moieties. Fluorescence can also be generated through a fluorogenic or chromogenic reaction such as enzymatic based (for example, HRP, alkaline phosphatase (AP), Tyramide Signal Amplification, antibody-based, etc). FRET, de-quenching or fluorescence enhancement can also be modulated in a molecular beacon, hairpin, aptamer, or nucleic acid enzyme molecule due to conformational change upon binding to a target analyte. In further embodiments, light-emitting moieties such as fluorophores are conjugated to, complexed with, or encapsulated in particles (or beads, emulsions, or vesicles) where each resultant particle is barcoded with a distinct lifetime signature. These particles can then be used to label the target molecules and be measured and analyzed through time-resolved techniques.

In some embodiments, additional parameters about light-emitting moieties and/or their surrounding environment including for example the characteristics of light-emitting moieties, distance and/or structural and architectural relations between light-emitting moieties, quenching or de-quenching of light-emitting moieties, energy transfer or charge transfer between light-emitting moieties, molecular rotation, as well as local environmental factors (for example pH, solvent, temperature, ions) can be used to modulate lifetime signatures in probe lifetime barcoding strategies. In further embodiments, provided are methods comprising adding, mixing or incubating the probes to the samples for staining, binding or hybridization and their related steps or processes. For fixed samples, permeabilization of cell membranes may be performed with agents including for example non-ionic detergents such as Triton X-100 or by physical means (for example mechanical, electrical, or sonication) to disrupt the cell membranes. For live or living cells, tissues or organisms, it should be understood a chemical, biological or physical delivery system including for example a virus, liposome, lipid, polymer, nanoparticle, protein, albumin, gel, injector, or catheter may be used to deliver the probes into the target cells or tissues. People skilled in the art can also perform various sample processing steps such as digestion with proteases, washing with detergents, using hydrogels to link to the tissue, etc. to further clear up the sample for staining and imaging. For probe staining or binding, such as oligonucleotide hybridization for FISH or antibody/protein binding for protein imaging, a series of steps can be performed. The staining, washing, mounting and imaging buffer, media or solvent often contain agents or additives including, but not limited to, formamide, sulfolane, butyrolactone, ethylene carbonate (EC), valerolactam, 2-pyrrolidone, dextran, dextran sulfate, polyethylene glycol (PEG), E. coli tRNA, Herring sperm DNA, Salmon sperm DNA, cot-1 DNA, bovine serum albumin (BSA), fetal bovine serum (FBS), saline sodium citrate, Tris, magnesium, antifade compounds such as phenylenediamine, propyl gallate, Sudan Black B, azo dyes, or sodium borohydride to facilitate specific biomolecular binding, reduce nonspecific binding and autofluorescence, or optimizing conditions for imaging. Additional factors such as probe concentration, temperature, salt and metal ion type and concentration, and pH can be adjusted to optimize staining and imaging performance.

In alternative embodiments, provided are protein imaging methods where the target is a protein, peptide, or an epitope. The said target protein can be a monomer, homodimer, heterodimer, or a multi-unit complex or structure. In alternative embodiments, the said protein can be modified, through for example post-translational modifications or can be a recombinant protein. In this case, the probes share similar design principles as described previously for nucleic acid detection. Therefore, the concepts and embodiments described above regarding light-emitting moieties, probe design and modification, labeling, lifetime barcoding, and signal amplification can all be applied herein for protein detection. In alternative aspects, protein detecting probes comprise a protein-binding motif including, but not limited to, nucleic acids, substrates, ligands, modified nucleic acids, receptors, proteins, antibodies or antigen binding fragments thereof (for example, Fab fragments) (and their various derivatives such as nanobody or single-chain variable fragment (scFv); see for example, Arlotta et al, 2019. Antibody and antibody derivatives as cancer therapeutics, 11:e1556), antibody-oligonucleotide conjugate, carbohydrates, aptamers, peptides, engineered tag or any combination of these molecules and their modified counterparts. Like nucleic acid probes as described previously, antibodies or antigen binding fragments thereof (for example, Fab fragments or single-domain antibodies (sdAb), also known as nanobodies) and other protein-binding moieties can be chemically or enzymatically synthesized or recombinantly expressed. The probes can bind to the same or different epitopes on the same protein or protein complex. In alternative aspects, at least one set of probes are covalently or non-covalently bound to light-emitting moieties. In alternative aspects, primary probes tethered with light-emitting moieties are used to directly label the target proteins. In other cases, indirect labeling schemes may be used where secondary probes tethered with light-emitting moieties are complexed with the target through adaptor molecules or readout domains on the primary probes, as described previously. In alternative aspects, probes are labeled on the “products” of protein target-mediated event or amplification including for example proximity ligation mediated padlock RCA. In alternative aspects, the protein-binding probes comprise antibody-nucleic acid conjugates or their derivatives for example antibody-oligonucleotide conjugate, or scFv-oligonucleotide conjugate. In alternative embodiments the said nucleic acid may comprise various functional domains such as barcode domain, PCR primer binding, adapter ligation domain, readout, etc. In the case of antibody-nucleic acid conjugate probe, all the concepts and embodiments we discussed in the previous nucleic acid probe sections including for example combinatory lifetime probe barcoding and labeling and signal amplification can all be applied here.

In further embodiments, the concepts disclosed here can be used alone or in combination for simultaneous or sequential detection of different types of species such as nucleic acids and proteins on the same sample. For instance, simultaneously imaging both protein and nucleic acid targets in the same sample can provide enriched information compared to either alone. In other cases, the concepts disclosed here for spatial profiling of biomolecules can be used in conjugation with existing methods in multimodal analyses. A few examples of such cases include, but not limited to, FLIM FISH analysis as provided herein for mRNA detection together with conventional immunohistochemistry (IHC), other immunolabeling or CytoTOF for protein detection or other sequencing-based methodologies. Such simultaneously or sequentially measure protein (or peptide, epitope) and transcriptome levels or other factors in the sample can provide enriched information for biology or disease diagnosis.

Time-resolved imaging and analysis: To detect, image, and discriminate the different types of probes or probe combinations including for example those outlined in FIG. 2 that may be used to label the target(s) of methods as provided herein that employ time-resolved measurements and analyses. One example is fluorescence lifetime imaging microscope (FLIM) that measures the time of arrival of individual photons upon excitation. It is important to note that this type of lifetime-resolved imaging may supplement or be used in combination with any other type of biophotonics imaging approaches including, but not limited to, intensity, amplitude or spectral based fluorescence measurements, superresolution imaging, light sheet microscopy, expansion microscopy, fluorescence (lifetime) correlation spectroscopy (FLCS), Fluorescence (lifetime) Cross-Correlation Spectroscopy (FCCS), Fluorescence Anisotropy (Polarization) and time-resolved fluorescence anisotropy, fluorescence (lifetime) fluctuation correlation spectroscopy(FLCS), second harmonic generation (SHG), and Coherent anti-Stokes Raman Scattering (CARS). For example, to image and identify a labeled target, the target may be measured by FLIM, spectral imaging, and/or polarization to discern its identity after correlating it to a codebook. Through this potential integration of imaging (though integration may not be used for certain targets), many multiplexing capabilities can be achieved that transcend the few or limited number of channels of conventional intensity-based conventional epifluorescence or confocal fluorescence microscopy.

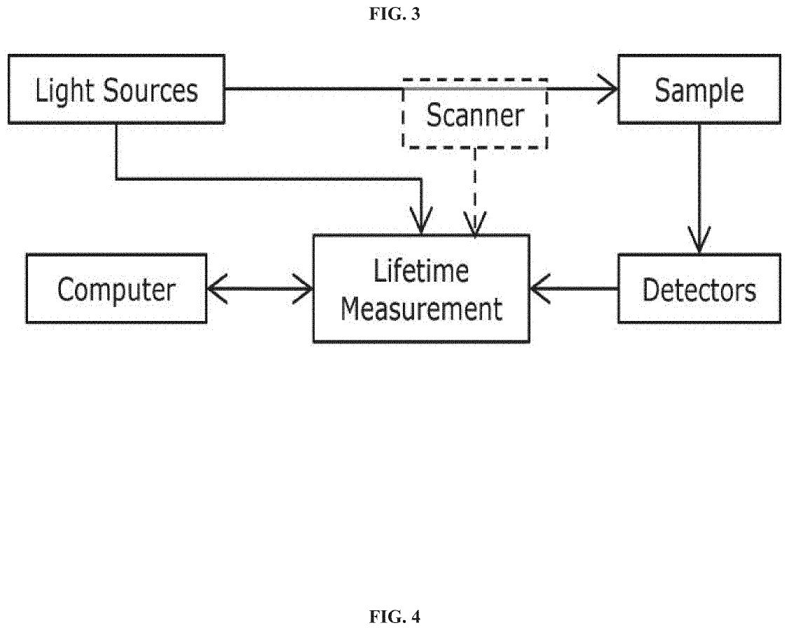

In one aspect, to utilize the said lifetime-resolved measurement, a standard microscope (for example scanning, wide-field), equipment, apparatus or instrument either a commercially available or a custom-built (an exemplary scheme is presented in FIG. 3) may be used which can be comprised of a light source to excite the sample and a set of detectors to collect the light emitted by the sample. Often, light source(s) excite the sample that are stained with probes and the emission is collected by the detector(s)/camera(s). Individual photon counts are registered by the lifetime imaging electronic device which in turn uses the clock from the light source to establish the time of arrival of the photons with respect to the excitation. If a scanner is used, it also provides a trigger signal to synchronize the spatial origin of each photon.

A detailed description of each exemplary component is presented in following sessions. In the case of lifetime imaging, the light source may be modulated or pulsed. In another embodiment, a light source may not be needed if the target is labeled with bioluminescent labels which can generate a fluorescence signal without an excitation source. Furthermore, detectors which may be used in this setup send signal counts to an electronic board that is coupled to or synchronized with the light source in order to precisely measure the arrival time or phase delay of the emitted light with respect to the excitation light. In another embodiment, this type of imaging can be conducted on a scanner-based instrument where the scanner may also provide a time stamp that allows to spatially locate the emission of each photon and therefore allows to compose images. In alternative aspects, this type of imaging can be conducted on a portable instrument. Several examples of the embodiments which these instruments are illustrated in J Biomed Opt. 2017 October; 22(12):1-10. doi: 10.1117/1.JBO.22.12.121608 “Portable fluorescence lifetime spectroscopy system for in-situ interrogation of biological tissues” or Rev Sci Instrum. 2014 May; 85(5):055003. doi: 10.1063/1.4873330 “A portable time-domain LED fluorimeter for nanosecond fluorescence lifetime measurements”. Several available lifetime imaging units including for example commercial electronic boards that may be used to acquire FLIM images, such as the FLIMbox by ISS, the SPC family by Becker & Hickl or the Harp boards (Time-Correlated Single Photon Counting (TCSPC) and Multi-Channel Scaling (MCS) board) by PicoQuant, and can be coupled to a commercial or a home built microscope. Alternatively there some available commercial microscopes that are integrated with lifetime capability such as the ISS ALBA or the Leica SP8 Falcon that may be used as well.

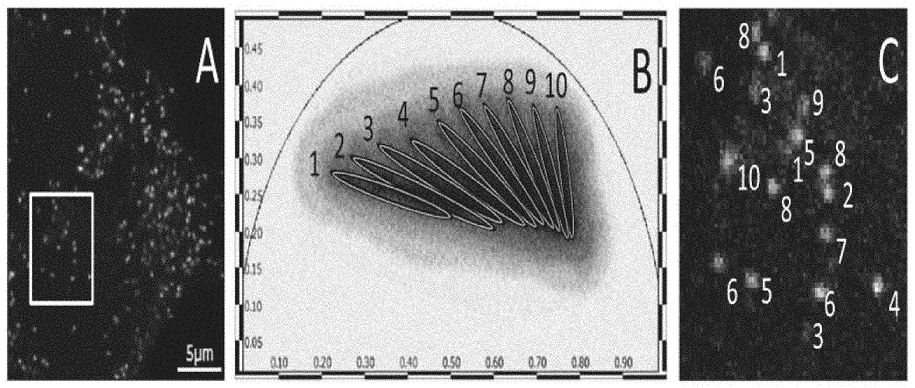

Lifetime analyses: Probes that are labeled with different characteristic fluorescence lifetime can be simultaneously excited and resolved by means of lifetime imaging and downstream analyses. In this manner, lifetime imaging multiplexes the number of unique targets detected by a single or multiple detector(s). The lifetime can be detected, analyzed, resolved, quantified, and presented in a manner to serve as metric(s) to indicate the presence of biological molecule(s) or process(es). The lifetime can be quantified as time delay of the emission from an excitation pulse or as phase and/or amplitude modulation of the emission correlating to the excitation light. Multiple algorithms or mathematical models including for example phasor approach, principal component analysis, time domain model fitting, fitting single, multiple, or a continuum of exponential functions, calculating the half-life of the lifetime decay or averaging photon arrival times, can be used for lifetime data analyses. These methods allow to extract from the measured decay and to estimate the lifetime(s), and optionally the creation of a color-mapped image where different colors represent different fluorescence decays or lifetimes. In one particular embodiment, the different lifetime populations can be resolved by fitting different decay models to the distribution of photons or by using the fit-free phasor approach to lifetime imaging (Ranjit, et al. 2018. Fit-free analysis of fluorescence lifetime imaging data using the phasor approach. Nat Protoc 13, 1979-2004). In one aspect, the phasor plots for fluorescence lifetime analysis serve as a graphical or visual representation of (fluorescence) lifetimes and-or combinations of lifetimes in one same pixel. The phasor approach maps each of the pixels in the image to a position to a two-dimensional space based on the measured histogram of the photon arrival times at the pixels. Briefly, by accumulating many photons at a single pixel, one can build a distribution of arrival times in that pixel as a function of the time between pulses (or the modulation period). This curve has a rise time that is due to the impulse response of the system and a decay which is due to the lifetime(s) of the molecules that are being excited and are contributing to that pixel. The phasor transform captures the shape of this curve by extracting two quantities from this curve that in turn define a position in the phasor space, namely the sine (S) and cosine (G) components for the time domain approach or the modulation and phase for the frequency domain. Phasors do not require data fitting, i.e. without requiring prior assumptions on the decay model. The phasor space is extremely useful because one can visually resolve different, heterogenous lifetime populations (as exemplified in FIG. 4). Note that the phasor-mapped FLIM image and the phasor plot can be cooperatively related using reciprocity iterations (i.e. feedback between phasor and image data) and optimize lifetime encoding probe design and decoding schemes. In addition, a set of algebraic rules can used on the phasor plot that allow manipulating the data and figuring out contributions of different components to the photon histogram of that pixel (“multi-component analysis”).

While this component separation in a single pixel is currently limited to two components, but also provided herein is a set of techniques based on obtaining the higher harmonics of the phasor transform, that allow resolving 2, 3, 4 and possibly more components in a single pixel. This greatly increases the throughput of the imaging since by using this method one can image much greater regions and a posteriori resolve the lifetime/spectral components present in individual pixels. The FLIM-phasor segmentation approach has traditionally been performed by hand i.e. manually selecting regions or populations in the phasor plot but here we show that we can apply machine learning and/or artificial intelligence-based approaches to segment the images in the phasor plot. The phasor approach to analyze lifetime and or spectral data can be performed using software such as the SimFCS software (as described in for example, Ranjit et al. Nature Protocols (2018) vol 13:1979-2004, or see https://www.lfd.uci.edu/globals/) that can be used to interface hardware such as FLIMbox or TCSPC. In alternative embodiments, other transformed subspace or approach for lifetime analysis of targets are employed, and methods are not limited to just the phasor approach.

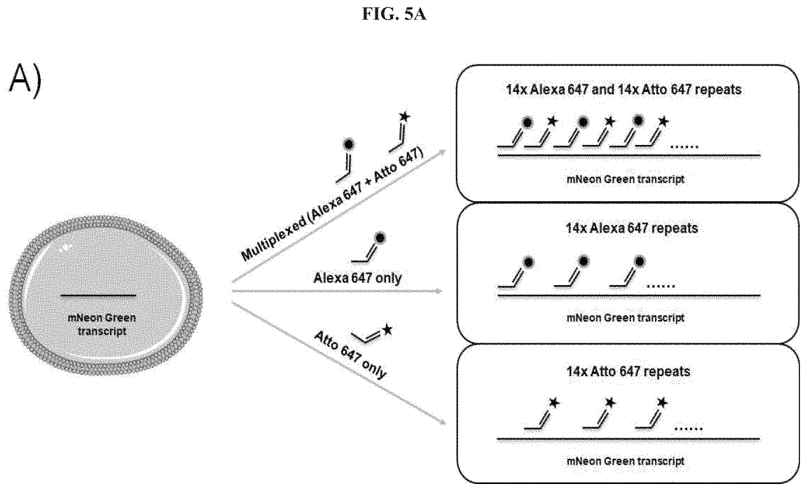

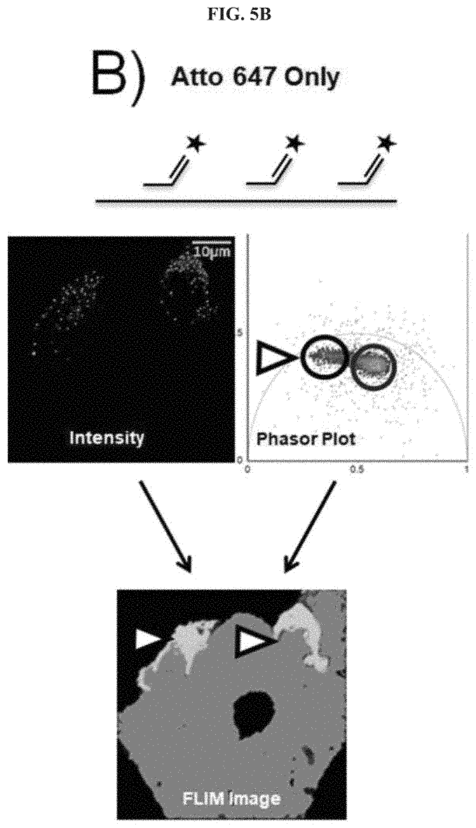

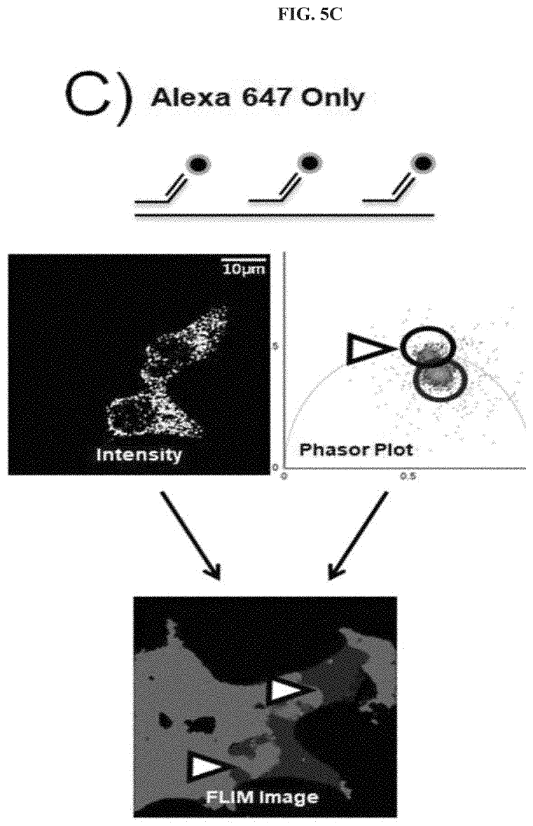

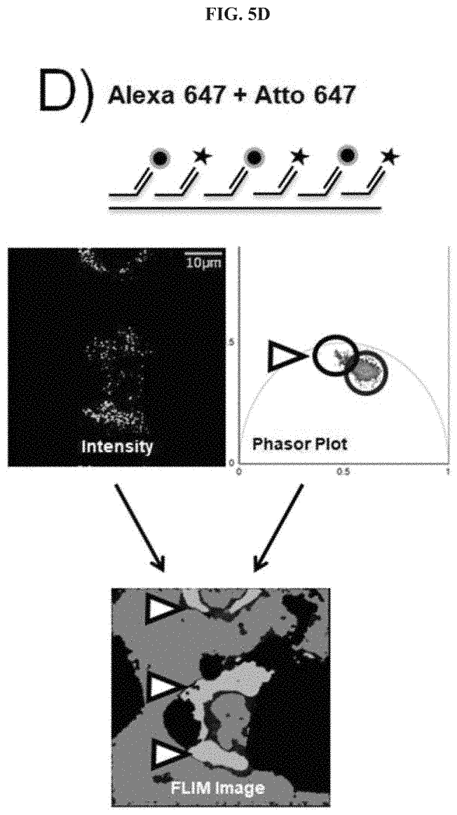

Autofluorescence: The lifetime analysis such as the phasor approach to lifetime imaging is a powerful tool that can be used to separate autofluorescence and background light components in the samples. This is due to the fact that the characteristic lifetime is in general different depending on the source in the sample and is therefore mapped to a different position on the phasor plot. By masking the areas on the phasor plot that are known to be autofluorescence one can greatly enhance the signal-to-noise ratio of the resulting images. An illustration of this can be seen in FIGS. 5, 6, and 7. Autofluorescence compounds or components may also be identified in this way and can be encoded in a codebook to allow the user of methods as provided herein to know the populations of components which are already existing in the sample prior to any labeling.

FLIM FRET: Lifetime imaging and phasor plot-based segmentation can be combined with Förster resonance energy transfer techniques and other lifetime probe barcoding strategies presented previously in order to enhance the multiplexing capabilities. In the case of FLIM FRET, for example, the concept relies on the fact that the excited state of one molecule (donor) can be transferred to a neighboring molecule (acceptor) in turn changing the lifetime of both donor and acceptor. This allows to map the spatial proximity of the two molecules on the phasor plot including FRET efficiencies and therefore to detect specific combinations of probes that are spatially connected. While FRET also changes the fluorescence intensity of both donor and acceptor and can be used to determine FRET efficiencies, the fluorescence intensity depends on many other factors such as excitation light intensity, sample refractive index, probe orientation, etc. Lifetime does not depend on such factors and can reliably quantify FRET efficiency in complex systems such as cells or tissues. Excitation Frequency: The lifetime can be measured in two ways: the time domain and the frequency domain. In general, the time domain approach (using for example time-correlated single-photon counting or TCSPC) relies on exciting the sample with a train of pulses whereas the frequency domain (analog or digital) relies on exciting with intensity-modulated light. The emitted photons from the sample are captured and the time of arrival of each of them is correlated to the excitation. The excitation frequency of the light source is a crucial aspect depending on the characteristic lifetime that one is measuring. For the case of the time domain approach, the excitation frequency is the inverse of the time between excitation pulses. For the frequency domain approach, the excitation frequency is the inverse of the period of the light source modulation. This frequency can be tuned so that in each cycle there is sufficient time for the probes to emit the fluorescence or phosphorescence, typically in the kHz-MHz region, optionally in the 1 Hz-1 GHz range, 10-100 MHz range, or 1 MHz-1 GHz range, allowing to capture lifetime emission in the ranges from about is down to about 1 ps. In the specific case of the slower lifetime phosphorescence, the frequency is in the 10 kHz-1 MHz region. In general, in order to capture faster decay, one must increase the excitation frequency and in order to capture slower decays one must reduce the frequency. In the time domain approach, a function is fitted to the decay curve obtained by accumulating many pulses, whereas in the frequency domain approach the phase shift between the excitation and the emission is measured.

Light Sources: To practice the disclosed methods, one may or may not need an external light source. In alternative aspects, for example, bioluminescent moieties can emit light without excitation from an external light source. In one aspect, some light-emitting moieties (for example fluorescent proteins) or light-emitting or chromogenic reactions as provided herein can serve as a light source to excite other light-emitting moieties. In other cases, exciting the probes is done by using a light source that will provide the required energy to excite the particular probes that are being used and that can be modulated as described in the previous section. The wavelengths used can be anywhere in the range from the ultraviolet to the infrared depending on the sample that needs to be excited. Two-photon excitation can also be used in which case the excitation light should have approximately twice the wavelength of the corresponding single photon excitation line. The light sources include but are not limited to lasers, laser diodes, fiber optics, LEDs, synchrotron radiation, mercury vapor lamps, xenon arc lamps, gas discharge lamps or incandescent lamps. In alternative aspects, one uses a specific laser line to guarantee a narrow wavelength band line but also one can select particular bands from a white light laser with interference filters or an acousto-optical filter, or an acousto-optical beam-splitter. Alternatively, one can use a very broad band and excite all probes with a single light source. In alternative aspects, the light in a FLIM setting is temporally modulated or pulsed. This can be done using intrinsically modulated laser source or by additional modulators. For example, a laser (for example Ti:Sa laser) emitting short, periodic, pulses can be used as the excitation light source. Furthermore, in the frequency domain, the demodulation of fluorescence emission and phase shift upon excitation with a high-frequency, periodically modulated illumination pattern (for example, pulse, rectangular, sine) can be measured, optionally by cross-correlation techniques. In alternative aspects, the light is polarized. While some lasers are locked to a specific pulse repetition rate, for example, 80 MHz for most Ti:Sa lasers, other lasers, especially diode lasers, can be modulated in a range of 0-340 MHz.

Multiphoton excitation: The fluorescence/phosphorescence process can be triggered by multiphoton excitation. In such cases, simultaneous absorption of more than one photon occurs such that the combined energy matches that of the required absorption of the molecules. This can be useful in order to excite many probes that have different absorption spectra while at the same time lowering the energy exposure of the sample, especially when imaging three dimensional stacks of images to avoid photobleaching of neighboring slices. Furthermore, multiphoton excitation unlike conventional confocal microscopy does not photobleach regions surrounding the point spread function (PSF) allowing one to fully image and capture large 3D sections of for example tissues, organs, organisms, etc. with minimal or negligible photobleaching. Multiphoton excitation also allows the penetration of the laser to overcome scattering effects to effectively image deeper into tissues beyond tens of microns which conventional fluorescence microscopy is restricted by. In some embodiments, utilizing multiphoton excitation techniques and their adaptations such as Deep Imaging Via Emission Recovery (DIVER) can permit millimeters thick tissues to be imaged with minimal limitations. (Dvornikov et al. 2019. The DIVER Microscope for Imaging in Scattering Media, Methods Protoc. 2, pii: E53. doi: 10.3390/mps2020053).

Detectors: The (emitted) light may be collected by means of detectors such as photomultiplier tubes, avalanche photo-diodes, single photon sensitive detectors (for example Single Photon Avalanche Diodes, SPADs), photodiodes, microchannel plate detectors, hybrid detectors and camera based instruments. Any number of detectors/cameras can be used provided that the corresponding beam splitters are used accordingly to the probes used. In general, the detector(s) comprise sufficient temporal resolution (for example picosecond resolution for nanosecond fluorescence lifetime) to resolve modulated signal information. Each detector need not be assigned to a particular probe, for example, one can sequentially excite the probes using the same detector to collect the emission. In addition, one can use pulsed-interleaved excitation (PIE) methods that can synchronize multiple pulsed lasers and can increase the imaging throughput by avoiding having to image sequentially or the use of different detectors for acquisition in different spectral ranges. This is done by intercalating pulses of different wavelengths and by matching the collected photons at the detector to the wavelength of each particular pulse. Using a lifetime camera to image large fields of view simultaneously can also increase the throughput as opposed to using a scanning microscope with a detector. Furthermore, various filters may be utilized such as any commercial dichroic filter, filter wheel, or acousto-optic tunable filters (AOTF) to permit detection of a specific or variety of different emitted wavelengths sequentially or simultaneously. In some aspects, the detector can encode and decode the signal information to be detected, optionally, in the temporal domain. In alternative aspects, the emitted signal can be patterned or modulated for detection. In another embodiment, lifetime is measured using a time-correlated single photon counting (TCSPC) system or a (multi)frequency phase fluorometer (MPF). In further embodiments, lifetime can also be measured and acquired using fast electronics (for example field-programmable gate array (FPGA)) combined with sensitive spectral hybrid detectors (for example spectral single-photon counting detectors) such as those found in the Leica SP8 FALCON system that enable (ultra)short dead time for data acquisition and analysis. In other aspects, mechanisms for example heterodyning that can convert high frequency signal to lower frequency using cross-correlation can be used. In alternative aspects, methods as provided herein use digital electronics that are capable of acquiring data using a digital parallel (multifrequency) acquisition mechanism or digitize recorded photons with separate comparators.

Super-resolution imaging: In alternative embodiments, super-resolution techniques, which are a family of microscopy techniques that allow measurements to surpass the diffraction limit due to the wave-nature of light which is around 250 nm, are also used. This is of relevance in order to spatially resolve individual targets at high resolution. Some of the techniques that can be used in conjunction with the disclosed time-resolved measurements include, but not limited to, stimulated-emission depletion microscopy (STED), structured illumination microscopy (SIM), photo-activated localization microscopy (PALM or FPALM) and stochastic optical reconstruction microscopy (STORM). STED is an example of a super-resolution technique that on one hand does not hinder the imaging throughput because it does not require additional imaging nor heavy post-processing components or demands and on the other hand can be combined with lifetime imaging to further resolve species. It relies on using a secondary laser source that follows the excitation pulse which depletes the excited state of the region around the focal volume. The depletion consists in stimulating a transition to a different state other than the natural decay to the ground state. Since this occurs in the immediate vicinity of the confocal volume this spatially decreases the region that is emitting fluorescence. This improves greatly the resolution in the imaging and allows for a more robust detection and counting of individual puncta structures. For the particular case of STED, since the depletion laser reduces the number of molecules in the excited state, that is manifested as a reduced lifetime of the fluorescence of the particular probe, this means that combining STED with lifetime imaging even further enhances spatial resolution of the images. Furthermore, methods as provided herein can employ any combination of these super resolution approaches with each other or any aforementioned biophotonics techniques such as spectral imaging, etc.

Spectral and Hyperspectral Imaging: In alternative embodiments, (Hyper)spectral imaging, which is a family of techniques that consist in separating the emission according to wavelength, is also used. It can be performed by splitting the emission and using multiple detectors or an array of detectors to collect the photons. In one embodiment, spectral imaging may permit targets labeled with fluorophores to be distinguished with up to 5 nm separation from another fluorophore. For instance, a target labeled with Alexa 565 can be distinguished from a target labeled with Alexa 560. In addition, the spectral-phasor approach is analogous to the FLIM-phasor in that it is a transformation to a phasor space where one can separate populations in order to remap back to the original image for segmentation. A cost-effective and fast approach that can be applied is to use a single detector coupled with a couple of orthogonal filters such as the sine-cosine (Dvornikov and Gratton, 2018. Hyperspectral imaging in highly scattering media by the spectral phasor approach using two filters. Biomed Opt Express. 9, 3503-3511). It is important to note that methods as provided herein can employ spectral imaging or any combination with spectral imaging and the aforementioned biophotonics techniques.

In alternative embodiments, the combination of lifetime and hyperspectral imaging are used to practice embodiments as provided herein. By utilizing both time and spectral domains for labeling and imaging, the multiplexing capability is further increased by discriminating a vast repertoire of lifetime and spectral components simultaneously within the same pixel or image of a sample. Combined hyperspectral and lifetime detection can be accommodated on both single point scanning systems such as confocal and multiphoton scanning microscopes and camera-based systems such as widefield epifluorescence and light sheet microscopy.

For scanning systems, for example, spectral separation can be achieved by splitting the light into multiple spectral channels (e.g. 8-256 channels) with a dispersive optical element such as a prism or grating, or, alternatively, detected in the same channel with tunable filters. With high speed detectors or detector arrays (e.g. 32 channels) such as photomultipliers, avalanche photodiodes and PIN photodiodes, the lifetime can be measured in each channel to complement the hyperspectral information. Together with a pulsed/modulated light source for excitation, the time delay between excitation and re-emission of a fluorescence photon can be determined in several ways including time domain lifetime measurements, frequency domain lifetime measurements, and the digital heterodyning approach for FLIM as utilized by FLIMBox. The exemplary spectral FLIM (S-FLIM) method as provided herein combines true parallel Digital Frequency Domain (DFD) electronics with a multidimensional phasor approach to extract detailed and precise information about fluorescent specimens at high (single pixel, submicron) optical resolution. This technology allows blind unmixing of spectral and lifetime signatures from multiple unknown species and unbiased bleedthrough-free and background-free FRET analysis in cells and tissues. DFD has the advantage that the bins of the fluorescence decay histogram are always a submultiple of the laser frequency. No error propagates within the mathematical operation when converting such histogram into phasors, whereas many parameters have to be taken into consideration and determined a priori when using the time-domain decay. DFD uses much less resources in a Field Programmable Gate Array (FPGA), resulting in higher scalability and highly parallel systems.

In alternative embodiments, DFD laser scanning systems as provided herein comprises 32 true parallel channels based on FPGA to obtain S-FLIM data. As an example, excitation can be achieved with a 78 MHz pulsed white light laser successively divided into parallel and independent laser lines (e.g. 405 nm, 488 nm, 561 nm, 647 nm, etc.) while the emission light can be passed through a pinhole for confocality and collected by the 32 channel spectral detector. The phasor approach can be computationally implemented by means of Fast Fourier transforms (FFT) by replacing the iterative search needed for fitting the decay with a single, parallel FFT over all points of the image. This approach allows for real time processing of S-FLIM images which is not achievable by any other computational technique without exploiting parallelization or GPU acceleration.

An exemplary setup of lifetime and hyperspectral imaging systems as provided herein comprise a custom confocal microscope with Spectral Fluorescence Lifetime Imaging Microscopy The excitation light is provided by a 78 Mhz pulsed White Light Laser (WLL, NKT Photonics SuperK Fianium FIU-15), the light is successively polarized in the direction parallel to the plane of the microscope by a laser grade polarizing beam splitter (PBS, Thorlabs) while the light perpendicularly polarized is discarded to a beam trap. The polarized light is then filtered to discard the far red and infrared light by means of a 670 long pass dichroic (LP 670, Chroma), selecting only the wavelengths in the range 400-670 nm. The visible light is separated in four spectral bands by means of a cascade of three long pass filters (LP 458, LP 525, LP 584, Chroma), each of which is then sent through a rotating linear polarizer (RLP, Thorlabs) for attenuation and successively through the appropriate band pass filter (BP 405, BP 488, BP 561 and BP 647, Semrock Laser Line). Finally, the laser lines are recombined using the same long pass dichroics used above, polarized by a rotating Glan-Thompson polarizer in the direction parallel to the plane of the microscope and sent to the main dichroic (DM 405), the scanning mirrors (SM, Cambridge Instruments), a scanner lens (SL) and the microscope body (Nikon TE2000-U) where it reaches the sample. Emission light is then transmitted through the main dichroic and is focused into a pinhole wheel (PH, Thorlabs) by means of an achromatic doublet (AD) and then refocused to either an Avalanche Photodiode (APD, Excelitas) or to the S-FLIM detector (spectrograph A10766, Hamamatsu) that has embedded a diffraction grating and an array of 32 photomultiplier tubes (H7260, Hamamatsu) for parallel spectra acquisition. The H7260 detector array is connected to an interface board (SIB232, Vertilon) which sends the signal through a high speed SAMTEC cable, connected to a custom designed PCB to separate this output in 32 separated SMA connectors. From the SMA connectors, the signals are split in two blocks of 16 (channels 1-16 and 17-32) through SMA to LEMO cables to two fast constant fraction discriminators (MCFD-16 Mesytec), each of which outputs a strip cable with the signals as LVDS (definition of this abbreviation). These cables are then sent to a LVDS to LVCMOS converter chip (SN75LVDS, Texas Instruments) and are finally collected from the FPGA (Xilinx XC7K325T-2FFG900C, Kintex-7 KC705 Evaluation Kit) where the DFD acquisition program is loaded. The FPGA also collects signals from the white laser for reference and from the scanning mirrors for reconstructing the image. The data are finally sent to the computer, for example, by means of controller, for example, using a USB 3.0 Controller (EZ-USB FX3™ SuperSpeed Explorer Kit—Cypress), and processed, for example, by a custom routine written in MATLAB R2019b. The scanning and the detection with the APD are controlled by the SimFCS software (Globals).

For camera-based systems such as light sheet or single/selective plane illumination microscopy (SPIM) the aforementioned sin/cos filters can be used to simultaneously collect hyperspectral information with a FLIM camera such as multi-tap complementary metal oxide semiconductor (CMOS) camera sensors or conventional cameras in combination with fast-switching image intensifiers. In alternative embodiments, the said SPIM system can be a sideSPIM (see e.g., Hedde, P. N., et al., sideSPIM-selective plane illumination based on a conventional inverted microscope. Biomedical Optics Express, 2017. 8(9): p. 3918-3937. 32. Hedde, P. N., et al., Ultrafast phasor-based hyperspectral snapshot microscopy for biomedical imaging. bioRxiv, 2020. DOI:10.1101/2020.10.14.339416) that comprises large, scalable sample chambers and high numerical aperture (NA), long working distance objective lenses to image samples as large as 8×8×3 mm³. In alternative embodiments, from visible or near infrared (NIR, for 2-photon excitation, 700-1000 nm) light, a light sheet can be formed at the focal plane of the detection lens from the side. Phasor-based hyperspectral snapshot microscopy performs the phasor transformation directly in hardware by passing the light though a pair of optical filters with transmission spectra in the form of a sin/cos period in the desired wavelength detection range. After sin/cos transformation of the emissions, each image pixel/voxel can be represented on a polar plot (phasor plot) with the angular position (phase) determined by the center of mass of the emission and the distance from the center (modulation) determined by the width of the spectrum. With a FLIM camera (for example, pco.FLIM, PCO) and laser modulation (1 MHz to 640 MHz) fluorescence lifetimes can be measured simultaneously. In alternative embodiments, an image splitter (e.g. MultiSplit V2, Cairn Research) can be integrated to simultaneously image all three parts (sin and cos filtered and unfiltered images) with the same camera. In alternative embodiments, a custom sample chamber can be integrated with the said light sheet imaging system to accommodate multiple samples. In alternative embodiments, image stacks are acquired by sample z translation with a motorized stage.

S-FLIM for spatial multi-omics imaging and analysis: By utilizing the lifetime and spectral domains for labeling and imaging this highly scalable approach can avoid multiple rounds of tissue processing and mechanical tissue sectioning and is expected to profile large tissue resections and whole biopsies (for example, approximately 8×8×3 mm³) within hours. The phasor approach can be used to rapidly and precisely measure and analyze fluorescence spectra and lifetimes. Representation of the lifetime decay data and hyperspectral data on the phasor plot allows the precise measurement of many spectral and lifetime components simultaneously without the need for computationally intensive and error-prone fits of the decay data in each pixel. In alternative embodiments, phasors for every single channel and pure components retrieved from the Phasor S-FLIM unmixing algorithm can be used to resolve multiple spectra and/or lifetime components. This “multicomponent analysis” is a powerful tool to ensure fidelity of target detection in spatialomics and to decode many different barcodes from different targets within the same diffraction-limited voxel. By quantifying the contributing fractions of fluorescence from each individual probe in each pixel we can achieve both high spatial resolution (single molecules) and high throughput for large tissue imaging. This approach can be used for spatial multi-omics combinatorial fluorescence hyperspectral and lifetime imaging using laser scanning or high throughput camera imaging such as light sheet microscopy. Such 2D or 3D spatial multi-omics technology integrates in situ labeling of molecular markers (e.g. mRNA, proteins) in tissues with combinatorial fluorescence spectral and lifetime encoded probes. When combined with light sheet imaging or other high throughput microscopy method, this combinatorial labeling enables rapid, high-plex spatial profiling of multi-omics biomarkers in cells, organoids, 3-dimensional (3-D) cell culture, thick tissue resections, and whole biopsy samples.

S-FLIM for Förster Resonance Energy Transfer (FRET): Different FRET pairs and their distances can be readily modulated to elicit changes in both spectral and lifetime to further enable greater multiplexing for spatial multi-omics imaging. The FRET phenomena with its nanometer-level sensitivity can resolve correctly target-bound dye-bearing strands from other nonspecific fluorescent puncta in the same or adjacent pixel/voxels. In Phasor S-FLIM, we have access to both the spectral and the lifetime information and we can combine it with the blind unmixing algorithm presented below. This combination will simultaneously account for both the bleedthrough and the unknown background contribution and can provide an unbiased and background-free multi-parameter analysis of FRET efficiency without the need for spectral calibration and with a much higher photon yield. The Phasor S-FLIM unmixing algorithm removes every contribution which may stem from unknown background (e.g. autofluorescence) or spectral bleedthrough which is always present when using emission filters. Direct excitation of the acceptor and the spectral overlap with the donor can be simply accounted for by using the Phasor S-FLIM approach. This feature can extend the applicability of precise FRET measurements for spatial multi-omics imaging to human tissue samples as well as to animal models.

Image processing and analysis software: In alternative embodiments, methods as provided herein further comprise an image processing component which can permit automatic analysis and identification of targets from a static or time-lapse 2D image or 3D z-stack. In one embodiment, automatic segmentation of individual targets in an image can be performed in the resulting two and/or three-dimensional images consists in detecting each individual structure. This provides a detailed morphological study of the spatial distribution of a particular target on top of counting the number of detected structures. Image segmentation methods that can be used include but are not limited to simple thresholding, morphological operations of the blob-detection family, statistical- or clustering-based methods, watershed-based methods or image segmentation methods based on graph theory. This can be done in individual images or z-stack slices or it can be done on a whole z-stack volume. If individual slices are segmented, each puncta can then be associated from one frame to the next using clustering methods including hierarchical-, centroid-, distribution- and or density-based methods. Segmentation of individual cells in a culture or in a tissue or particular cell organelles such as the nuclei can be performed to select relevant regions of interest or in order to provide statistics in units of density (for example per area, per cell) of puncta instead of absolute counts. Furthermore, the analysis in the phasor space, both spectral and lifetime imaging phasor, can be done automatically by means of segmentation methods such as watershed-based, thresholding-based, supervised or unsupervised clustering or combinations of segmentation and clustering such k-means or gaussian mixture model. Neural network based, machine learning or Artificial Intelligence (AI) methods also can be used to interpret the phasor space and convolutional neural networks in order to identify populations in the phasor space.

An example of an embodiment which this software program can take is a customizable MATrix LABoratory (MATLAB), R, python, C, etc. script which allows a user to input their intensity-based and lifetime-based images for automated image processing and analysis. Inputting images with measured intensity and lifetime data into this program may allow the software to register and phasor transform each pixel photon arrival time and spectra for a position on the phasor plot subspace. Afterward, populations of pixels with distinct lifetimes and spectra in this subspace may be resolved and segmented automatically based on the chosen fluorophores used in the experiment. An example of this can be seen in FIG. 4B. Furthermore, each population on the phasor plot may correspond to a different target and may be isolated and represented as a unique mask with a distinct spectra or lifetime-based labeled signature. These unique mask or channels can then be outputted as a regular TIF, JPG, PNG, etc. file. The algorithms employed in this software to permit automatic clustering on the phasor plot and for resolving independent lifetime/spectral species in single pixels may be based on standard image processing techniques such as watershed-based, thresholding-based, supervised or unsupervised clustering or combinations of segmentation and clustering such k-means or gaussian mixture model combined with phasor algebra. It is important to note that these standard image processing techniques may be done manually with open source software such as MATLAB, R, python, C, etc. since these functions are inherently built in these softwares.

In alternative embodiments, phasor algebra is manually done via publicly available software such as simFCS (Laboratory of Fluorescence Dynamics, https://www.lfd.uci.edu/globals/) by pre-measuring and assigning unique linear combination or combinations of fluorescent signatures to certain phasor positions in the subspace and then corresponding them to a certain target. The algorithm used for phasor algebra for two to three components which reside in the same pixel has been outlined in multiple papers and an example can be found here (Ranjit, et al. 2018. Fit-free analysis of fluorescence lifetime imaging data using the phasor approach. Nat Protoc 13, 1979-2004).

Since these manual processes are not automated and requires technical training or skill in various disciplines, methods as provided herein can automate this process by integrating them together in a certain sequence or combination to preclude the need for any expertise and allows user to attain decoded identification of a vast repertoire of uniquely labeled targets. Furthermore, in principle, the linear combination rule is valid for any arbitrary number of components or fluorescent signatures but becomes more difficult for more than three components per pixel due to the need to cleanly resolve each component from each other. In alternative embodiments, methods as provided herein use an algorithm which predicts every possible linear combination or combinations of fluorophores based on empirical testing, machine learning, and mathematical models. This algorithm may reliably resolve 10 to 20 components in one pixel for samples taken at high or poor resolution and allow greater multiplexing capabilities even when a sample is taken with a low magnification objective.

In alternative embodiments, these mask or channel files are processed via a commercial or open source software such as CellProfiler, imageJ, etc. (Carpenter et al., 2006. CellProfiler: image analysis software for identifying and quantifying cell phenotypes. Genome Biol. 2006; 7(10): R100) for automatic quantification of 2D and 3D images or stacks and x, y, and z spatial validation. These software can also remap the original image with each target highlighted with its corresponding unique shape or color code for target and spatial identification. In other embodiments, the outputted mask or channel files are fed into a default script, which may be written in MATLAB, R, Python, etc. which can fulfill the same function as these independent software but with more customizable features such as a bigger repertoire of shape and color code options as shown in FIG. 4C. In another embodiment, provided are integrated and customizable GUI for all these components for improved user friendliness. This integrated and customizable GUI may be written by us in MATLAB, R, Python, etc. or a third party GUI programs such as the 3D Project plugin function for Image J™. In some embodiments, all the analyzed data can be replotted in a different projection to illustrate all multiple 2D or 3D configurations of the data. The user may then select the targets of interest to project into this plot or deselect which targets that the user may not be interested in.

However, an embodiment where the user can select the type of label coding scheme which they would prefer to use in addition to the recommended default coding scheme as provided herein may be a customizable software program written in MATLAB or other languages by us. Furthermore, this GUI may allow users to manipulate this projection in real time for example inverting it, flipping it, etc. This GUI may also permit the user to perform or analyze the data in real time by allowing the user to manually gate the lifetime or spectral phasor with cursors such as the function which is available in the Leica LAS X software. This GUI program can also perform any statistical calculations or correlations such as Pearson Correlation or Area Under Curve (AUC) based on the type and number of targets present and detected on the sample as well as spatial positioning of the targets for bioinformatic analysis. This provided statistical analysis option is a component which we will build via MATLAB, R, python, etc. This GUI can also allow the user to export these data into any format of interest such as TIF, JPEG, excel, doc, etc. for personalized analysis. This software may also employ any data compression or transformation techniques to allow processing of data files with smaller sizes or in a different transformation to allow seamless manipulation of very large data files such as the Leica LAS X™ software.

In another embodiment, methods as provided herein comprise use of an integrated GUI for all these components for improved user friendliness. In this embodiment, all the analyzed data can be replotted in a different projection to illustrate all multiple two dimensional (2D) or three dimensional (3D) configurations of the data. The user may then select the targets of interest to project into this plot or deselect which targets that the user may not be interested in. The user may then select the type of label coding scheme which they would prefer to use in addition to the recommended default coding scheme as provided herein. They can also manipulate this projection in real time for example inverting it, flipping it, etc. This GUI may also permit the user to perform or analyze the data in real time by allowing the user to manually gate the lifetime or spectral phasor with cursors. This GUI program can also perform any statistical calculations or correlations based on the number of targets present and detected on the sample as well as spatial positioning of the targets for bioinformatic analysis. This GUI can also allow the user to export these data into any format of interest such as TIF, JPEG, excel, doc, etc. for personalized analysis. This software may also employ any data compression or transformation techniques to allow processing of data files with smaller sizes or in a different transformation to allow seamless manipulation of very large data files.

### Products of Manufacture and Kits

Provided are products of manufacture and kits for practicing methods as provided herein; and optionally, products of manufacture and kits can further comprise instructions for practicing methods as provided herein.

Another set of embodiments provide kit(s) for detecting one or multiple target biological materials. For example, in nucleic acid detection, the kit comprises target nucleic acid(s), sets of primary probes (often oligonucleotides), optionally, a simple amplification component, and optionally, a set of secondary probes that stain the “readout” domains of the primary probes. The kit may also comprise various other components such as agents for sample fixation, permeabilization, hybridization, blocking, washing, buffering, mounting, etc. In another example, for protein imaging, the kit comprises target protein(s) or epitopes, sets of primary probes (often antibodies or antigen binding fragments thereof (for example, Fab fragments or single-domain antibodies (sdAb), also known as nanobodies) or their derivatives), optionally, a simple amplification component, and optionally, a set of secondary probes that bind to the primary probes or products of target binding-mediated event or amplification. The kit may further comprise various agents for example for sample fixation, permeabilization, hybridization, blocking, washing, buffering, mounting, etc. It should be understood that the combination of the above embodiments or multiple sets of kit components can be used together for multiplex detection.

### Computers and Computer Systems

Methods and computer program products as provided herein, for example, the algorithm or software as illustrated in FIG. 21 for target molecule (shown as “puncta”) detection and classification following spectral/FLIM imaging, can be practiced using computers and storage memory systems for performing the operations as provided herein. In alternative embodiments, this apparatus may be specially constructed for the required purposes, or it may comprise a general-purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions.

The algorithms and displays used to practice systems and methods as provided herein are not inherently related to any particular computer or other apparatus. Various general-purpose systems may be used with programs in accordance with the teachings herein, or it may prove convenient to construct a more specialized apparatus to perform the method steps. The structure for a variety of these systems will appear from the description provided herein. In addition, embodiments provided herein are not described with reference to any particular programming language. It will be appreciated that a variety of programming languages may be used to implement and practice methods and systems as described herein.

In alternative embodiments, data generated and processed by components of systems and methods as provided herein, include generated data and programs used to practice embodiments as provided herein, are stored and processed using a machine-readable medium, which can includes any mechanism for storing or transmitting information in a form readable by a machine, for example, a computer. For example, a machine-readable medium includes a machine-readable storage medium (for example, read only memory (“ROM”), random access memory (“RAM”), magnetic disk storage media, optical storage media, flash memory devices, etc.) and/or a machine-readable transmission medium.

In alternative embodiments, programs used to process methods and/or systems as provide herein are cloud-based and use wireless systems to communicate (for example, device-to-device (D2D) connectability) with a user (for example, an individual being treated using systems or methods as provided herein) and/or an operator (for example, a person monitoring and/or administering methods or systems as provided herein as they are being practiced, for example, as described in U.S. Pat. No. 10,834,769, which teaches methods by one or more processors for managing a wireless communication network and device-to-device (D2D) connectability.

In alternative embodiments, systems or methods as provided herein use cloud computing to enabling convenient, on-demand network access to a shared pool of configurable computing resources (for example, networks, network bandwidth, servers, processing, memory, storage, applications, virtual machines, and services) that can be rapidly provisioned and released with minimal management effort or interaction with a user or manager of systems or methods as provided herein.

In alternative embodiments, provided herein is a non-transitory, machine-readable medium, comprising executable instructions for practicing programs as provided herein, for example, as illustrated in FIG. 21, that when executed by a processing system including a processor facilitate performance of operations, the operations comprising a program used to practice methods or systems as provided herein.

In alternative embodiments, systems and methods as provided herein use handheld devices and/or Bluetooth transmissions to practice embodiments as provided herein, for example, as described in U.S. Pat. No. 10,834,764.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (2012) Molecular Cloning: A Laboratory Manual, 4th Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Combinatorial Fluorescence Lifetime Encoded Probes for Multiplexed Detection

This example further demonstrates that methods and compositions as provided herein can be used to provide multiplexed detection via combinatorial labeling, for example as depicted in FIG. 5.

To scale up multiplexing capabilities, this exemplary application comprises barcoding approaches that scale combinatorially (nCr), but crucially still only requires one imaging round. In one example, a combinatorial-based labelling approach using dual dye-bearing probes on different primary strands can sparsely sample the possible barcode combinations (for example, 140 out of a possible 560 combinations).

In this example, samples containing a mixed population of a) HEK293 cells transfected to express mNeonGreen transcripts and protein (mNeonGreen is a basic (constitutively fluorescent) green/yellow fluorescent protein), and b) HEK293 cells non-transfected and not expressing any mNeonGreen transcripts and protein were labeled. Three conditions using this mixed population of cells were tested. For each sample, the mRNA transcripts were first labeled with a set of 14 of primary probes. Each primary probe is comprised of a 20 to 35 nucleotides (nt) complementary region towards the target and a 20 to 35 nt readout region which can bind to a subsequent secondary probe. One sample (FIG. 5A middle) was labeled with primary probes which have a readout region complementary to only secondary probes with Alexa 647. Another sample (FIG. 5A bottom) was labeled with primary probes which have a readout region complementary to any secondary probes with Atto 647. The third sample (FIG. 5A top) was labeled with primary probes which have a readout region complementary to secondary probes that were conjugated to Alexa 647 or Atto 647. An example of one of these probes targeting the mNeonGreen mRNA transcript is 5′-CTCGACCTTTCTCTTCTTCTTGGGGCTTTTAGAGTGAGTAGTAGTGGAGT-3′ (SEQ ID NO:216) where 5′-CTCGACCTTTCTCTTCTTCTTGGGGCT-3′ (SEQ ID NO:217) is the complementary target region and 5′-AGAGTGAGTAGTAGTGGAGT-3′ (SEQ ID NO:218) is the readout binding region. An example of a readout probe used for the previously described mNeonGreen primary probe is 5′-Alex647/ACTCCACTACTACTCACTCT-3′ (SEQ ID NO:219).

FIGS. 5B, C, and D show intensity-based and FLIM images of these differentially labeled samples taken on a microscope with FLIM capabilities. Since both fluorophores (Atto 647 and Alexa 647) are excitable at the same wavelength but differ in lifetime properties, all the mRNA transcripts shown in the intensity-based images (top left for each section) appear the same in color but may be differentiated by lifetime. However, when taken with a FLIM microscope and analyzed using phasor plots, the images of each sample can be differentiated based on the population of pixels that correspond to each labeling condition. For the sample that was labeled with only Atto 647 (FIG. 5B), the targets elicited a fluorescence lifetime signature of only the Atto 647 fluorophores approximates 4 nanoseconds (ns). For the sample that was labeled with only Alexa 647 (FIG. 5C), the targets elicited a fluorescent lifetime signature of only the Alexa 647 fluorophores approximately 1 ns. For the sample that was labeled with both Alexa 647 and Atto 647 (FIG. 5D), the targets elicited a fluorescence lifetime signature of a linear combination or blend of both Alexa 647 and Atto 647 fluorophores approximately 2.5 ns. Labeling mRNA targets with a combination of fluorophores can thus elicit a unique blend, encoded, or barcoded fluorescence signature. Depending on which fluorophore is used, the particular fluorophores used will all contribute to a unique position on the phasor plot, allowing significant multiplexing capabilities to occur based on combinatorial labeling.

### Example 2: Fluorescence Spectrum and Lifetime Encoded Probes for Multiplexed Detection of Cancer Markers

This Example describes how compositions and methods as provided herein can be used to provide multiplexed detection of cancer gene expression target.

In this example, the cell colorectal adenocarcinoma line, SW480 or ATCC® CCL-228, is used. One group of SW480 cells has been treated to express the ROBO1+ gene while another group, ROBO1− has been treated to not express this gene. From single-cell sequencing results, these two groups have a different transcriptomics profile. However, methods and compositions as provided herein can be used to map their spatial locations and acquire absolute quantification of this transcriptomics profile (36 gene expression targets). Several representative examples from this gene list are: LGR5, PCDH19, SEMA3D, ROBO2, COLCA1, and DKK4. To carry out detection, identification, and quantification of this panel, the samples are first fixed with paraformaldehyde and permeabilized with some denaturing or proteolytic agents. The samples are then blocked and incubated in blocking buffer with primary DNA probes targeting the specific genes. Each gene has 40 primary probes that are about 60 to 110 nt in length. Each primary probe is comprised of an about 20 to 35 nt complementary region towards the target and two flanking about 20 to 35 nt readout region which can bind to a subsequent secondary probe. Two readout regions are used in this particular example to allow double the number of secondary probes to bind. In total, 1,440 primary probes are used (40 probes per gene target×36 gene targets). The primary labeled sample is then incubated with the complementary secondary labeled probes. The labeling used in this iteration utilizes combinatorial labeling where 9 different fluorophores are used in different combinations of two to permit 36-plex detection via intensity-based and lifetime-based measurements. These 9 fluorophores may be ATTO 488, BODIPY 488, ALEXA 488, ATTO 565, ALEXA 532, BODIPY 532, ALEXA 647, ATTO 647, and BODIPY 647. For each gene, 20 of the primary probes can be subsequently labeled with one fluorophore while 20 of the other 40 primary probes can be subsequently labeled with a different fluorophore. Using a combinatorial scheme where there are 9 available options with 2 selection options, 36 different multiplexing options can label each target uniquely. For example, one target can be labeled with BODIPY 488 and ALEXA 488 while another target can be labeled with BODIPY 488 and ALEXA 647. Each of these fluorophores for this particular scenario will differ by either intensity-based or lifetime-based property.

After labeling, this labeled sample is then measured with a microscope such as the Alba ISS with APD and FLIM capabilities. A 20 μm z-stack of a large 500 μm×500 μm confocal area will be measured. For each target which will be represented by a population of pixels that shares the corresponding intensity-based and lifetime-based property of that specific label. A codebook which has paired a particular labeling condition with the mRNA target is then used in supplement with an automated FLIM phasor segmentation software to reveal identity of all the detected targets. This software also allows the user to get number of copies per gene and the corresponding x, y, z locations for each target. Furthermore, antibody-based labeling will be used to identify the cells. These antibodies or antigen binding fragments thereof (for example, Fab fragments or single-domain antibodies (sdAb), also known as nanobodies) can utilize any of the fluorophores previously listed or utilize a fluorophore with a different intensity-based or lifetime-based property. Since the software can predict the expected structure based on shape predicting algorithms or screening for a specific intensity-based or lifetime-based property, the user is permitted to utilize antibody-based labeling with mRNA-based labeling simultaneously with no limitations.

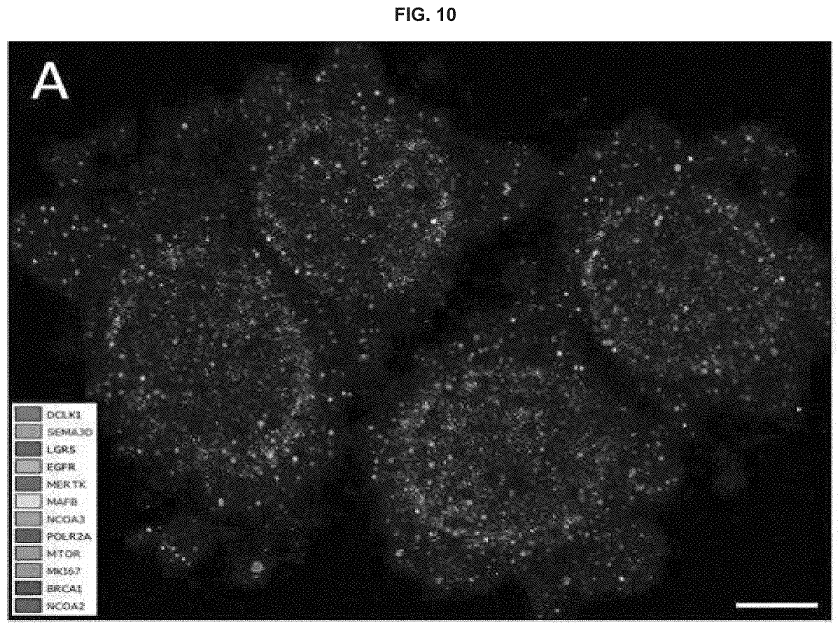

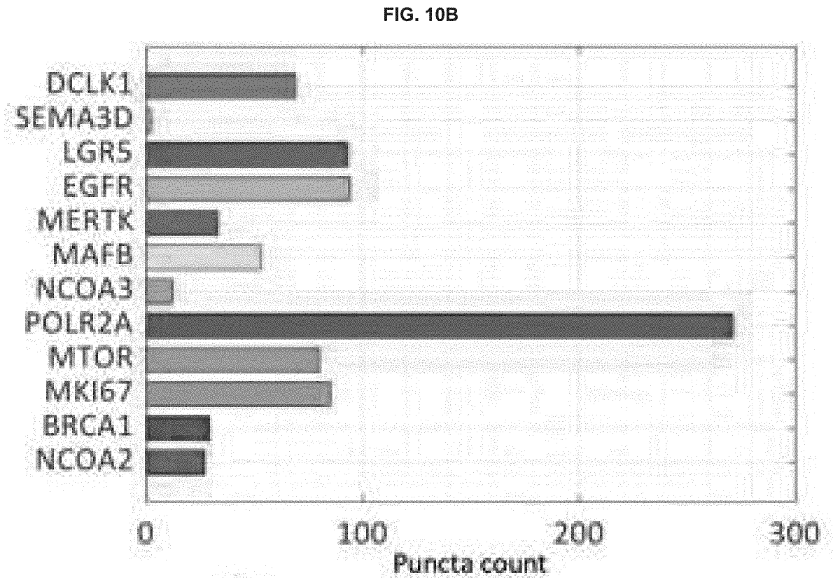

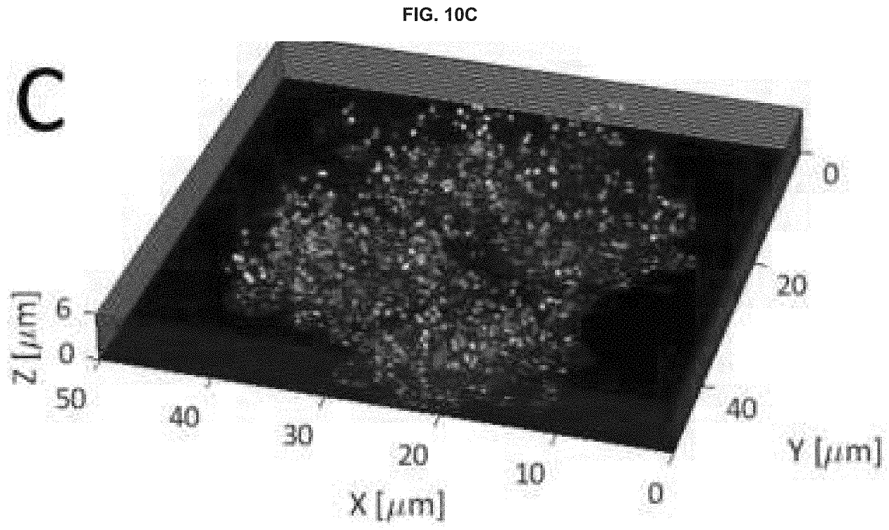

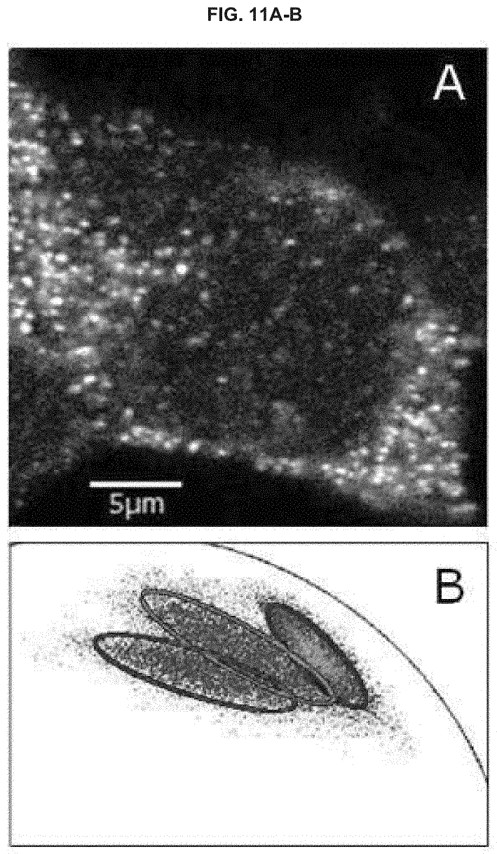

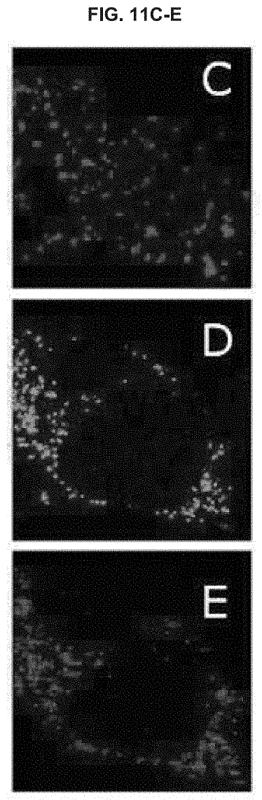

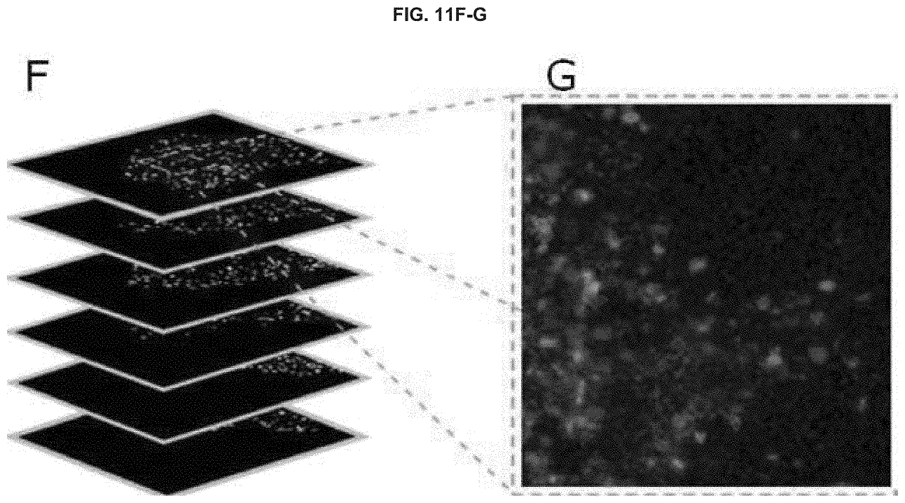

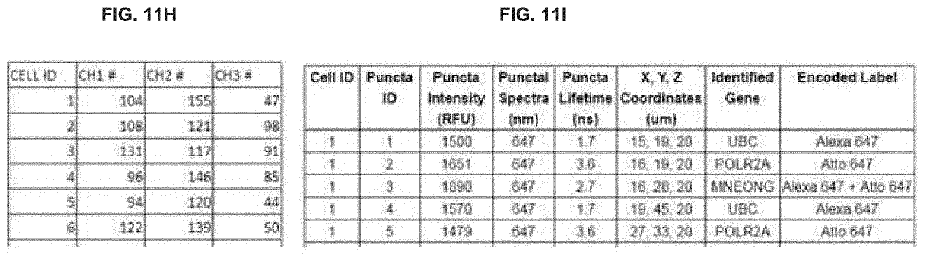

FIG. 10 further illustrates 12-plex mRNA detection with just one round of staining and imaging. Specifically, samples containing SW480 colon cancer cells which were sequenced and determined to express DCLK1, SEMA3D, LGR5, EGFR, MERTK, MAFB, NCOA3, POLR2A, MTOR, MKI67, BRCA1, and NCOA2 mRNA were labeled, imaged, and analyzed using an exemplary custom algorithm (as illustrated in FIG. 21) as provided herein (see FIG. 11). Briefly, for each detected puncta a set of features are measured including its intensity in each spectral channel and its lifetime phasor coordinates in each spectral channel. Using this set of features a decision tree classifier assigns the puncta to one of the 12 combinatorially labelled target genes based on the known spectral and lifetime characteristics of the fluorophores. Each gene was encoded to express a unique fluorescent signature which differed in either spectrum or lifetime property. With the exemplary combinatorial approach as provided herein, 6 secondary fluorophore probes can distinguish 12 different targets. For example, MERTK (ATTO 647) and MAFB (ALEXA 647), which exhibit the same spectral profile, can now be distinguished via FLIM, thereby increasing multiplexing.

### Example 3: Fluorescence Lifetime Encoded Probes for Multiplexed Detection of Brain, Neurological or Central Nervous System (CNS) Markers

This example describes how exemplary methods as provided herein can be used in neuroscience to, for example, provide multiplexed detection of Alzheimer's Disease (AD) related gene and protein expression is described below.

In this example, human Induced pluripotent stem cell (iPSC)-derived hematopoietic progenitor cells are transplanted into postnatal brains of immune-deficient mice as an investigation to study microglial homeostasis and disease-associated inflammatory responses. For histological analysis, the brain is fixed and prepared as formalin-fixed paraffin embedded (FFPE) sections. These tissues are then sliced into 40 μm sections and processed with the staining and clearing reagents and protocols described in this disclosure to label the protein and nucleic acids of interest. For cell species identification, the human protein marker, ku80 is labeled and detected to distinguish transplanted human cells from host mouse cells. For microglia identification, the protein markers, human leukocyte antigen ABC (HLA-ABC) and CD11b, are labeled and measured. Commercial directly conjugated antibodies or antigen binding fragments thereof (for example, Fab fragments or single-domain antibodies (sdAb), also known as nanobodies) to Alexa 647, Atto 647, or BODIPU 647 fluorophores can be used for each protein target. For mRNA transcripts labeling, a panel of 5 genes of interest i.e. Runt-related transcription factor 2 (RUNX2), MEF, JUN, FOS, Kruppel-like factors (KLF) are labeled to provide single cell transcript detection.

To carry out transcript labeling, the samples are blocked and incubated in blocking buffer with primary DNA probes targeting these specific genes. Each gene has 20 primary probes that are about 60 to 110 nt in length. Each primary probe is comprised of an about 20 to 35 nt complementary region towards the target and two flanking about 20 to 35 nt readout region which can bind to a subsequent secondary probe. Two readout regions are used in this particular example to allow double the number of secondary probes to bind. In total, 100 primary probes are used (20 probes per gene target×5 gene targets). The primary labeled sample is then incubated with the complementary secondary labeled probes. The labeling used in this iteration utilizes a simple labeling scheme where the primary probes for each target are labeled with only one type of fluorophore for 5-plex detection. These 5 fluorophores can be ATTO 488, BODIPY 488, ALEXA 488, ATTO 565, and ALEXA 532. Each of these fluorophores for this particular scenario differ by either intensity-based and/or lifetime-based property. A lifetime-based measurement can be particularly effective in this case to remove tissue autofluorescence that is known to be notoriously challenging for brain tissues due to autofluorescence from for example beta-amyloids and plagues.

The labeled protein and mRNA targets are then measured with a microscope such as the Alba ISS with APD and FLIM capabilities. A 40 μm z-stack of a large 2,000 μm×2,000 μm confocal area will be measured. For each target which will be represented by a population of pixels that shares the corresponding intensity-based and lifetime-based property of that specific label. A codebook which has paired a particular labeling condition with the mRNA target or protein target is then used in supplement with an automated FLIM phasor segmentation software to reveal identity of all the detected targets. Software as provided herein will then process the image to detect all the labeled targets based on fluorescence and shape matching algorithms or screening so that the user is permitted to utilize antibody-based labeling with mRNA-based labeling simultaneously with no limitations.

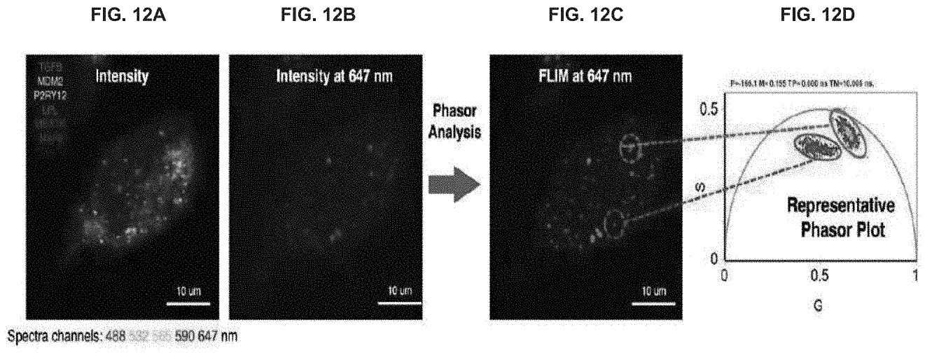

As illustrated in FIG. 12 as an example, samples containing microglia cells which were sequenced and determined to express TGFB, MDM2, P2RY12, LPL, MERTK, and MAFB mRNA were labeled, imaged, and analyzed using the exemplary approach as provided herein. Each gene was encoded to express a unique fluorescent signature which differed in either spectrum or lifetime property. Six types of puncta were simultaneously detected and revealed to directly match one of encoded signatures that was used (FIG. 12A). Importantly, MERTK (ATTO 647) and MAFB (ALEXA 647), which exhibit the same spectrum profile, can be distinguished via FLIM (FIG. 12B, C), thereby increasing multiplexing.

### Example 4: Target Quantification, Data Analysis, and Software

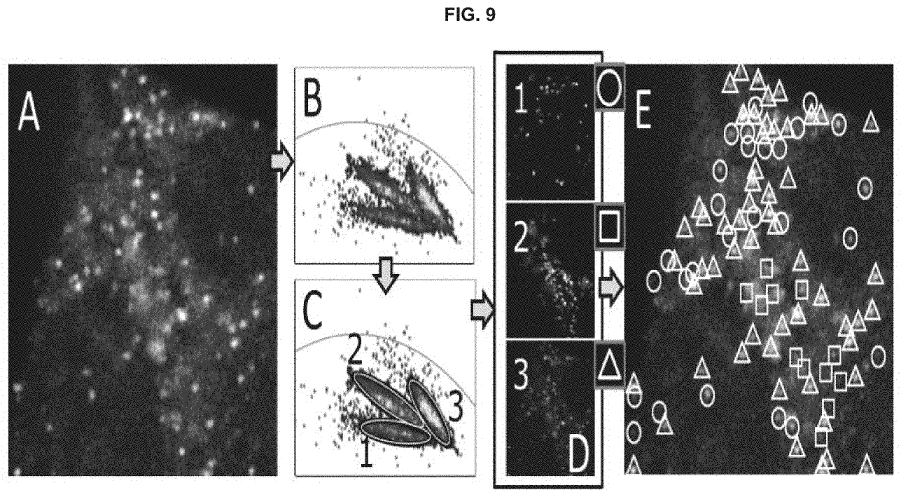

This example further describes an exemplary method comprising use of an automated phasor-FLIM target segmentation and counting software for mRNA target quantification and identification, as illustrated in FIG. 9.

As each target transcript is represented as “puncta” by a unique population of pixels that shares the corresponding spectrum and lifetime characteristics of that specific label, a codebook with a manual software tools such as simFCS or automated, custom-written, spectral and lifetime phasor segmentation software can be developed to reveal presence, identity, expression level, location, distribution, heterogeneity of each biomarker. User interface can be implemented using tools including for example HTML.

As illustrated in FIG. 21, the said software analyzes input data that are the individual photons detected by the system. In a general case, one has both the lifetime (time of arrival) and spectra (wavelength) of individual photons, although the method is also applicable in a reduced version with only spectral or only lifetime information available. Using the acquisition parameters, individual photons are assigned to each voxel (pixel for the reduced case of a single image) of the 3D stack. The resulting intensity stack (or image) is used to detect individual puncta based on local maxima detection or other traditional segmentation and blob detection algorithms. In parallel, the spectral/lifetime information in each pixel allows to construct a pixel photon distribution which is phasor transformed to obtain the pixel phasor coordinates. A machine learning clustering algorithm allows to cluster this data and relate the clusters to the expected populations based on the probes used to tag the sample. Joining the phasor information with the puncta detection, one obtains the spectral and lifetime coordinates of each puncta. These coordinates are used to classify each puncta into a particular label by means of a machine learning classifier. Finally, individual puncta morphological properties are measured, and the statistics (i.e. counts, frequency, density etc.) of the sample are obtained. By means of a cell segmentation procedure, statistics of individual cells are obtained as well.

In alternative embodiments, the input of this software is the Z-stack images and the parameters are the intensity threshold and puncta radius limit and shape morphology. Of note, puncta shape and size can be used to distinguish true positive from false positive signals resulted from autofluorescent moieties or non-specific probe binding. By collecting the x, y, z coordinates of our Z-stack images, we can reconstruct a 3D map of biomarkers of interest in the tissue. This exemplary algorithm further enables both protein and RNA detection. For protein analysis, it can determine whether a target protein is expressed, and if so, reveal its expression level, location, pattern, positive cell count, % positive population, and heterogeneity. To facilitate data analysis and presentation, we can also bin RNA and protein expression levels into “scores”. Our spatialomics data can be further visualized using e.g. standard t-SNE or UMAP plots and our custom imaging segmentation computational pipeline or others in the art such as CELLPROFILER™ (CellProfiler™), ILASTIK™ to classify and visualize single-cell phenotypes, their spatial organization and neighborhood relationship.

FIG. 11 illustrates how the phasor space and images can be iterated to enable automated detection and analysis of multi-omics biomarkers by intensity and lifetime signatures. Briefly, stacks of hundreds of images are taken from multiple combinatorial labeling experiments to serve as training sets for this software. These stacks are first screened for their spectral and lifetime components on a pixel to pixel basis to calculate the fraction of independent lifetime or spectral components. The meta data of each image are also processed to standardize differences in imaging settings such as hardware components, objective used, pixel depth, etc. Experiments utilizing single label transcripts vs. combinatorial labeled transcripts vs. fluorescent beads are imaged and tested against each other for optimization. Furthermore, these masks can be decoded by written scripts to reveal the number, location, identity, size, etc. of each multi-omic biomarker in addition to providing standard statistical algorithms to streamline analysis.

In this particular scenario, a sample containing HEK293 cells with three types of mRNA transcripts may be targeted: ubiquitin C (UBC), mNeonGreen, and DNA-directed RNA polymerase II subunit RPB1 (POLR2A). These cells may be grown on a covered chamber glass slide and then fixed with paraformaldehyde for subsequent labeling. Furthermore, they can be transfected with a mNeonGreen expressing vector to express mNeonGreen protein while UBC and POLR2A are inherently present as housekeeping genes. These mRNA targets can first be labeled with a set of primary DNA probes that varied in length from 40 to 70 nt. Each target may be labeled with 14 of these primary probes which is comprised of a 20 to 35 nt complementary region towards the target and a 20 to 35 nt readout region which can bind to a subsequent secondary probe. A representative example of one of these probes targeting the mNeonGreen mRNA transcript is 5′-CTCGACCTTTCTCTTCTTCTTGGGGCTTTTAGAGTGAGTAGTAGTGGAGT-3′ (SEQ ID NO:216) where 5′-CTCGACCTTTCTCTTCTTCTTGGGGCT-3′ (SEQ ID NO:217) is the complementary target region and 5′-AGAGTGAGTAGTAGTGGAGT-3′ (SEQ ID NO:218) is the readout binding region. Each mRNA target may have a corresponding DNA readout probe with a specific color in order to encode a unique color for target quantification and identification. An example of a readout probe that may be used for the previously described mNeon green primary probe is 5′-Alex647/ACTCCACTACTACTCACTCT-3′(SEQ ID NO:219). Three fluorophores all excitable with the same excitation laser but each containing a unique position on the phasor plot may be selected in order to allow separation and identification of these transcripts based on lifetime measurements.

FIG. 9A shows a representative image of this sample containing three types of mRNA transcripts labeled with a different fluorophore that may be taken on a microscope with FLIM capabilities. Since each fluorophore is excitable at the same wavelength and differs only in lifetime properties, all the mRNA transcripts shown in the image may appear the same. FIG. 9B shows how inputting this image into the exemplary program as provided herein can allow the software to register and phasor transform each pixel photon arrival time for a position on the phasor plot. FIG. 9C indicates how exemplary software based on the pre-measured and calibrated lifetime of the dye chosen can automatically segment the population of pixels that correspond to the particular fluorophore. In doing so, three population of pixels may be identified and gated. Furthermore, each population on the phasor plot may correspond to a different gene expression target and may be processed via a different mask allowing individual puncta to be detected and identified. FIG. 9E shows how the software can then remap the original image with each transcript highlighted with its corresponding unique shape or color code for target and spatial identification. A corresponding data file can then be outputted in any format such as excel to reveal the x, y, z spatial location of the transcripts as well as the total number of transcripts for that target and any statistics corresponding to this type of data.

### Example 5: Nucleic Acid and Protein Co-Imaging

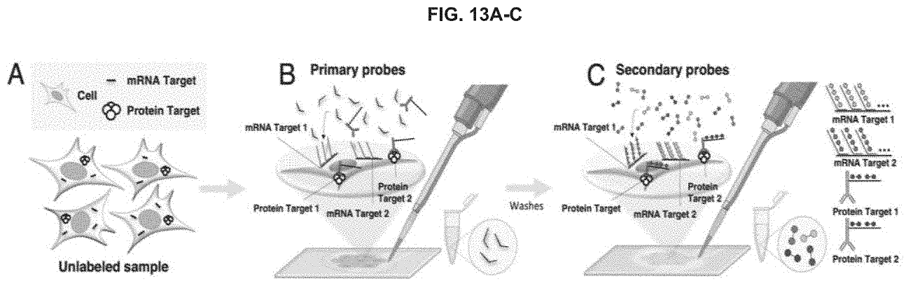

This example illustrates how methods as provided herein permit significant multiplexing capabilities for labeling, detection, identification, and spatial validation of two or more different species. FIG. 13 illustrates an example for simultaneous codetection of protein and nucleic acid targets.

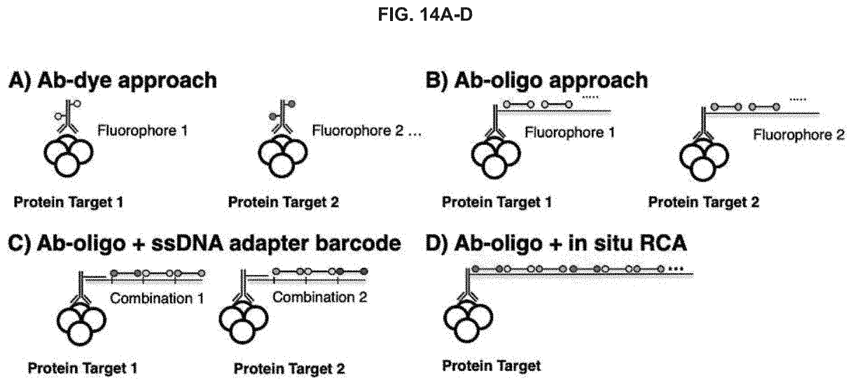

In this example, the sample is a mouse colon tissue containing 16 different cell types for example tuft, enteroendocrine, goblet, paneth, enterocytes, peyer patches cells, etc. For protein detection, each cell type has a unique and characteristic surface membrane marker or intracellular marker. Conventional fluorescence microscopy usually permits only four to five protein targets maximum to be detected at a time for example utilizing antibodies or antigen binding fragments thereof (for example, Fab fragments or single-domain antibodies (sdAb), also known as nanobodies) with fluorophores excitable at 400 nm, 488 nm, 546 nm, 647 nm, or 750 nm. In this scenario, methods as provided herein substantially enable analyses beyond this limited range by utilizing primary antibodies or antigen binding fragments thereof (for example, Fab fragments) conjugated with a fluorophore directed towards each target that differ from each other in their intensity-based, lifetime-based, or polarization-based properties. For example, to differentially label and detect the dendritic cells of the peyer patches from the goblet and paneth cells, an anti-CD14 IgG conjugated with Alexa 647 can be used for the dendritic cells while an anti-Mucin 5AC (MUC5AC) IgG conjugated to Atto 647 and an anti-DefensinA6 (DEFA6) IgG conjugated to BODIPY 647 can be used for the goblet and paneth cells, respectively. When imaged with a confocal microscope which FLIM capabilities at 647 nm excitation and in PBS, these three targets will be represented by a corresponding population of pixels with a characteristic lifetime based on the fluorophore used to label them. Dendritic cells will show up as a population of pixels with a lifetime around 1 ns while the goblet and paneth cells will show up as a population of pixels with a lifetime around 4 ns and 5 ns, respectively. In alternative embodiments, methods as provided herein use automated shape detection software to identify, quantify, and spatially validate all these 16 cells and provide an analyzed data output file for the user to use. Furthermore, in order to match the cell type with its transcriptomic profile, nucleic acids can be labeled simultaneously, detected, identified, quantified, and spatially validated to provide the transcriptomic profile of each cell type. For a panel of 64 genes, a combinatorial-based labeling method can be used to unique encode and label each mRNA target. After labeling and detection, the software can predict the expected structure based on shape predicting algorithms or screening for a specific intensity-based or lifetime-based property, the user is permitted to utilize antibody-based labeling with mRNA-based labeling simultaneously with no limitations. Furthermore, it should be understood that in the examples as provided herein, including this one, though maybe only several mRNA and/or protein markers are mentioned, the disclosed embodiments can profile 10s, 100s, 1000s or greater number of mRNA, even the whole transcriptome and/or 10s, 100s, 1000s or greater number of protein markers, even the whole proteome simultaneously using exemplary highly multiplexable lifetime encoding probe strategies. In addition, for protein detection, besides directly staining with antibody conjugated with fluorophores (FIG. 14A), antibody-nucleic acid conjugate probes can be particularly effective as the nucleic acid sequences can be utilized for efficient and high-degree spectrum and lifetime encoding using additional oligonucleotide probes including optionally a combination of different fluorophore-bearing strands to encode a combinatorial barcode for each target (FIG. 14B,C). Alternatively, the adapter molecule can be long ssDNA molecules generated using rolling-circle-amplification (RCA) (FIG. 14D) to enable signaling amplification, combinatorial barcoding and digitally counting of proteins.

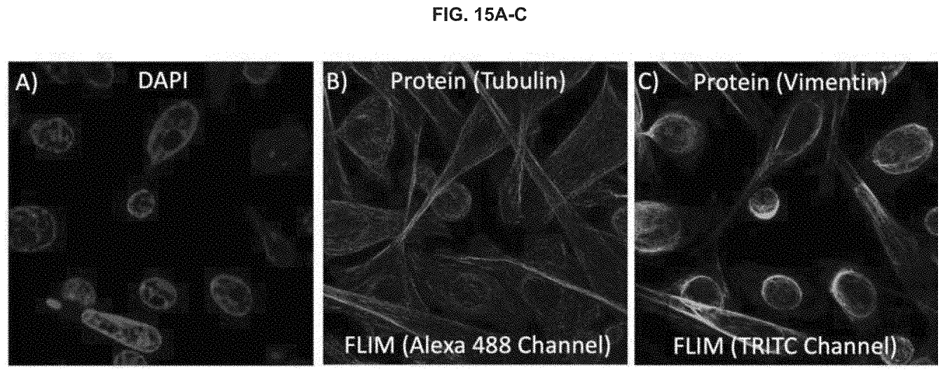

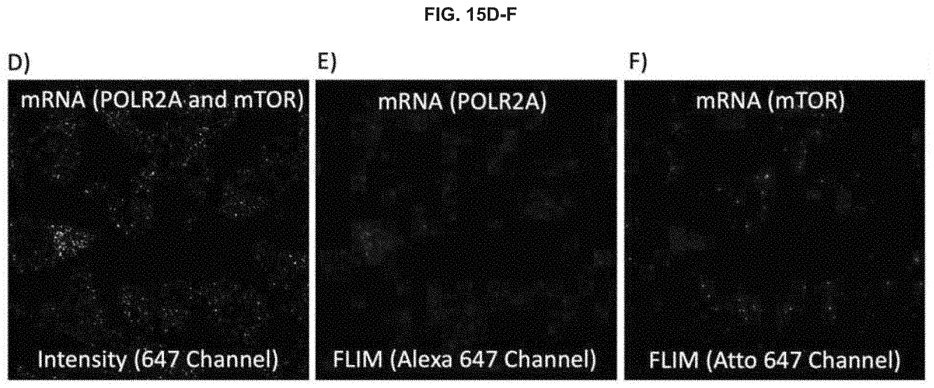

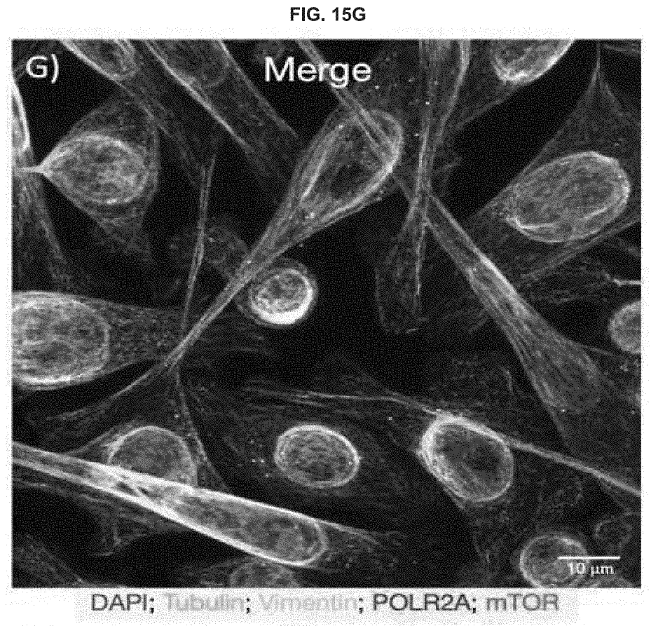

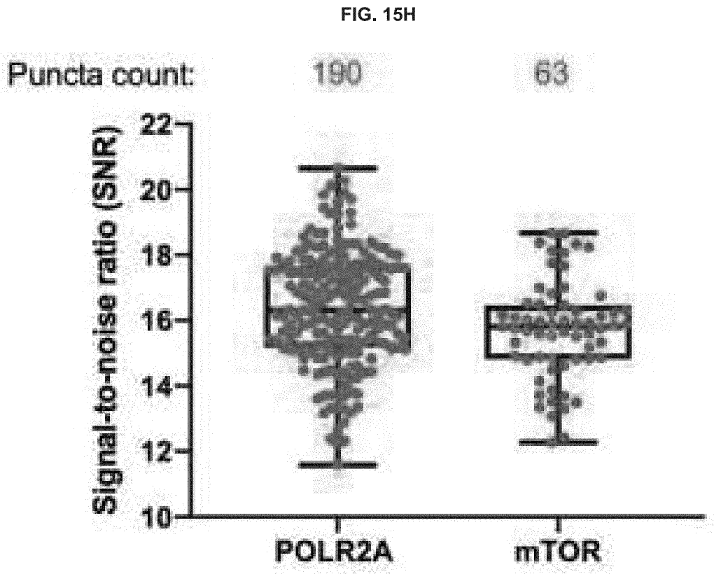

FIG. 15 further illustrates simultaneous 4-plex co-detection of protein (Tubulin and Vimentin) and mRNA (POLR2A and mTOR) in colon cancer SW480 cells using exemplary methods as provided herein.

### Example 6: Spatial Profiling of Biological Materials with Combined Super-Resolution Imaging

This example describes how super-resolution imaging approaches can be combined with labeling strategies as provided herein to improve detection resolution of targets.

In this particular example, the super-resolution technique, stimulated emission depletion (STED), was used. Here, a sample containing HEK293 cells with ubiquitin C (UBC) mRNA transcripts are labeled. UBC mRNA targets were first labeled with a set of primary DNA probes that varied in length from 40 to 70 nt. Each target was labeled with 14 of these primary probes which is comprised of a 20 to 35 nt complementary region towards the target and a 20 to 35 nt readout region which can bind to a subsequent secondary probe. An example of one of these probes targeting the UBC mRNA transcript is 5′-GAGGCGAAGGACCAGGTGCAGGGTGGATTTGGGATGTATTGAAGGAGGA T-3′ (SEQ ID NO:220) where 5′-GAGGCGAAGGACCAGGTGCAGGGTGGA-3′ (SEQ ID NO:221) is the complementary target region and 5′-GGGATGTATTGAAGGAGGAT-3′ (SEQ ID NO:222) is the readout binding region. These mRNA targets share a corresponding DNA readout probe with Alexa 647 in order to encode a unique color and lifetime for quantification and identification of this particular mRNA species. The complementary readout probe used in this example is 5′-Alex647/ATCCTCCTTCAATACATCCC-3′ (SEQ ID NO:223).

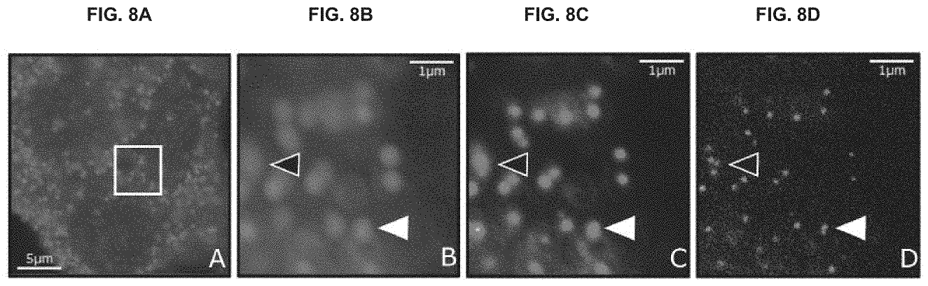

FIG. 8A shows an image of a sample containing UBC mRNA transcripts stained with Alexa 647 taken under regular confocal imaging. FIG. 8B depicts a region of interest from the same confocal image which will be compared against in STED conditions. FIG. 8C shows the same region of interest but with STED imaging. The arrows indicate how increasing the depletion laser strength leads to an increase in resolution. Puncta which appears as a single unit blob in the confocal image are revealed as groups of puncta in the STED conditions. Furthermore, FIG. 8D demonstrates how increasing the laser depletion power can lead to further improvement in resolution, allowing even further transcripts to be resolved from each other.

### Example 7: Detect mRNA in Optimum Cutting Temperature Preserved Samples

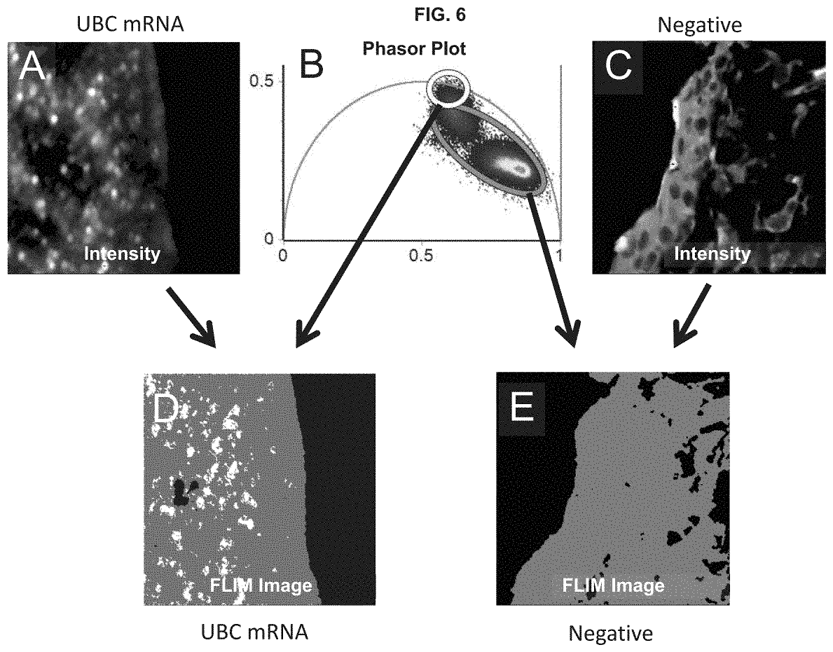

This example describes detecting mRNA transcripts in optimum cutting temperature (OCT) preserved mouse skin tissue is depicted in FIG. 6 using exemplary methods as described herein.

OCT is a common preservation method which allows samples to be stored fresh or fixed at freezing temperatures for long periods of time. Upon thawing at room temperature, these OCT tissues are immediately re-fixed to adhere the sample to the attachment substrate. Proper ensuing treatment of the sample is critical to maintain the integrity and proper labeling of the targets of interest present in the tissue. In alternative embodiments, provided are a series of methods and protocols which allow these samples to be processed effectively by using a certain set of denaturing and permeabilization reagents, temperatures, and incubation times in sequence. In this particular example, upon proper treatment, UBC mRNA transcripts from mouse skin tissue preserved via OCT medium were labeled with a set of primary DNA probes that varied in length from 40 to 70 nt. Each target was labeled with 28 of these primary probes which is comprised of a 20 to 35 nt complementary region towards the target and a 20 to 35 nt readout region which can bind to a subsequent secondary probe. An example of one of these probes targeting the UBC mRNA transcript is 5′-GAGGCGAAGGACCAGGTGCAGGGTGGATTTGGGATGTATTGAAGGAGGA T-3′ (SEQ ID NO:220) where 5′-GAGGCGAAGGACCAGGTGCAGGGTGGA-3′ (SEQ ID NO:221) is the complementary target region and 5′-GGGATGTATTGAAGGAGGAT-3′ (SEQ ID NO:222) is the readout binding region.

These mRNA targets share a corresponding DNA readout probe with Alexa 647 in order to encode a unique color for quantification and identification of this particular mRNA species. The complementary readout probe used in this example is 5′-Alex647/ATCCTCCTTCAATACATCCC-3′ (SEQ ID NO:223). FIG. 6A shows the resulting intensity image of a mouse skin tissue sample with transcripts labeled with Alexa 647 taken on the ISS Alba microscope with FLIM capabilities while FIG. 6C shows the resulting intensity image of a mouse skin tissue sample with transcripts not labeled with anything to serve as a negative control comparison. FIG. 6B shows the phasor plot of the pixels from both images 6A and 6C. When gating for the expected lifetime of Alexa 647, only the pixels constituting the labeled UBC mRNA transcripts in FIG. 6A are highlighted. As shown in FIG. 6C versus 6D, upon gating for only the expected lifetime of Alexa 647, only the targets which are labeled in FIG. 6A are correctly detected and identified while the targets which are not labeled in FIG. 6C are correctly not detected. Furthermore, when gated for any other lifetime, only pixels constituting the highly fluctuating autofluorescence background are highlighted. Indicating that background autofluorescence can be identified and separated from the fluorescent signatures emitted by the labeled targets.

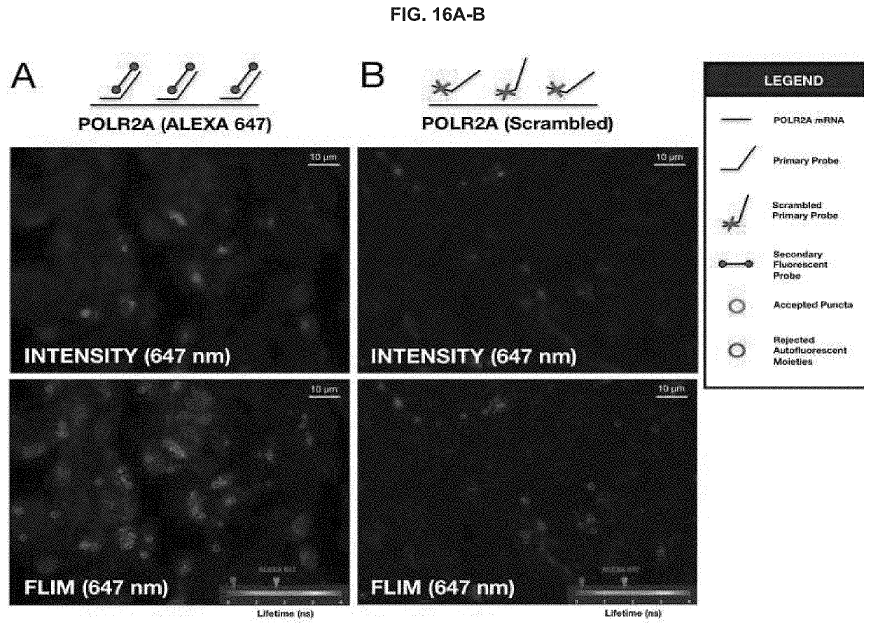

Furthermore, it was demonstrated that this exemplary method can work with highly scattering and autofluorescent tissues to remove sample artifacts and false positive moieties. As an example, we have demonstrated the detection of POLR2A transcripts in challenging matrices such as human skin tissues preserved in FFPE medium. Using standard intensity-based fluorescence microscopy, we could not differentiate between labeled puncta from autofluorescent moieties with similar SNR. However, with spectral-FLIM, background tissue artifacts (red circles) could be effectively subtracted out to reveal distinct puncta (green circles) which directly matched the encoded fluorescent signature (FIG. 16).

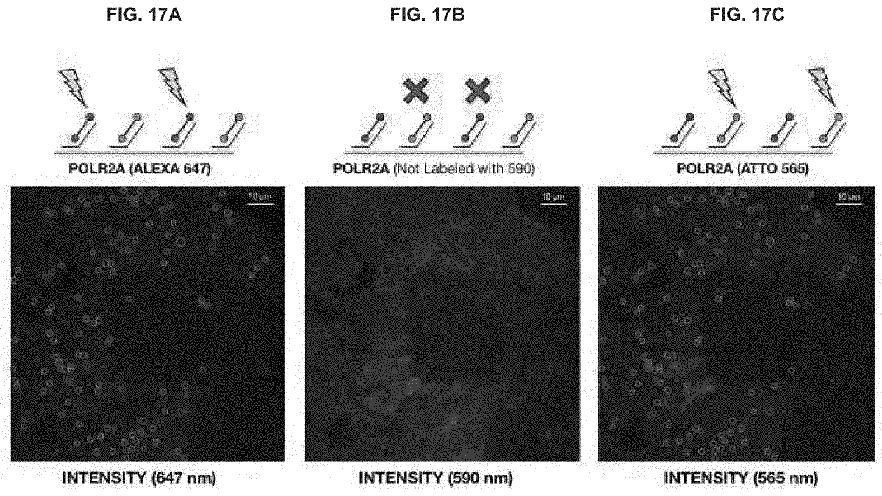

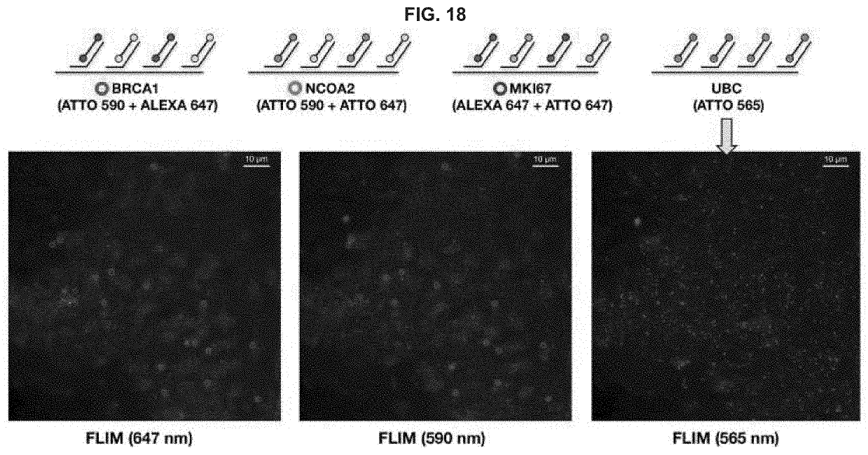

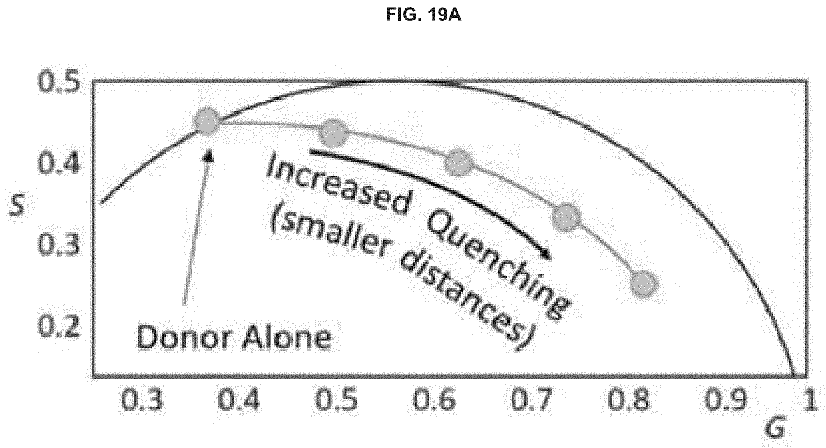

To further improve detection efficiency and implement error correction, we utilize combinatorial labeling by labeling targets with two or more dyes that are spatially colocalized. As an example, we demonstrated the detection of POLR2A transcripts labeled with ATTO 565 and ALEXA 647 (FIG. 17) with POLR2A puncta (green circles-puncta appearing in both 565 nm and 647 nm channel) being separated from autofluorescent moieties with similar SNR (red circles-only in a single channel) in human skin FFPE tissue. To demonstrate that targets were labeled specifically and appeared only in the intended channel(s), we imaged the 590 nm channel (in between the 2 target channels) and did not detect target puncta. We implemented this combinatorial labeling approach on a panel of 4 mRNA targets (BRCA1, NCOA2, MKI67, and UBC) in human skin FFPE tissue (FIG. 18). Puncta that appeared in their assigned combinatorial channels were circled and classified as the target puncta while autofluorescent moieties were removed.

### Example 8: Detect mRNA in Formalin-Fixed Paraffin-Embedded (FFPE) Samples

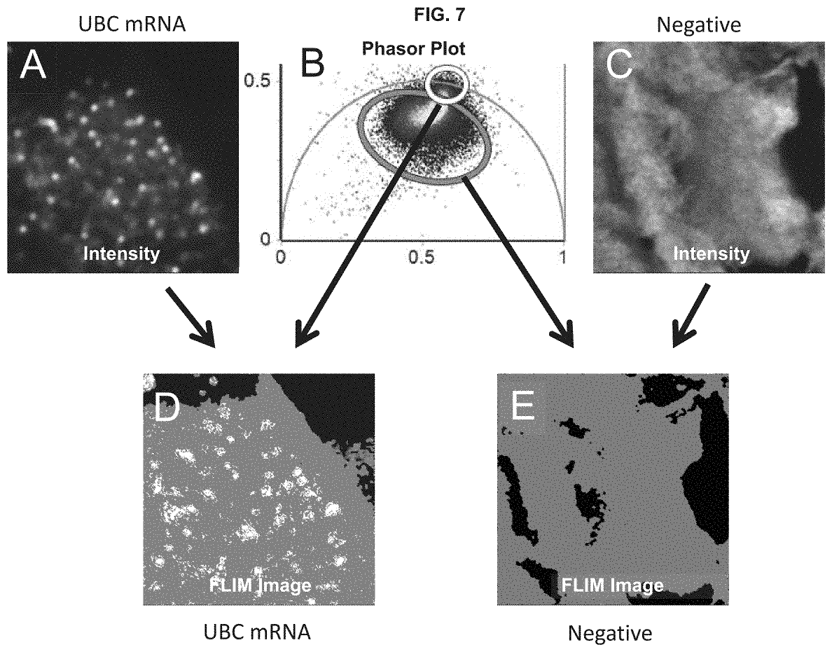

This example describes detecting mRNA transcripts in formalin-fixed paraffin-embedded (FFPE) preserved mouse colon tissue is depicted in FIG. 7 using an exemplary method as provided herein.

FFPE is a preservation method used often in the clinical setting for storing patient tissues because it allows samples to be stored at room temperature for long periods of time up to a decade. In this particular example, FFPE tissues were sectioned in 10 μm slices and were adhered to electrostatically rendered glass slides via paraformaldehyde fixation as well as high temperature incubation. Proper ensuing treatment of the sample is critical to maintain the integrity and proper labeling of the targets of interest present in the tissue. In alternative embodiments, provided are a series of methods and protocols which allow these samples to be processed accordingly by using a certain set of denaturing and permeabilization reagents, temperatures, and sequence. Upon proper treatment, UBC mRNA transcripts from the FFPE preserved mouse colon tissue were labeled with a set of primary DNA probes that varied in length from 40 to 70 nt. Each target was labeled with 28 of these primary probes which is comprised of a 20 to 35 nt complementary region towards the target and a 20 to 35 nt readout region which can bind to a subsequent secondary probe. An example of one of these probes targeting the UBC mRNA transcript is 5′-GAGGCGAAGGACCAGGTGCAGGGTGGATTTGGGATGTATTGAAGGAGGA T-3′ (SEQ ID NO:220) where 5′-GAGGCGAAGGACCAGGTGCAGGGTGGA-3′ (SEQ ID NO:221) is the complementary target region and 5′-GGGATGTATTGAAGGAGGAT-3′ (SEQ ID NO:222) is the readout binding region. These mRNA targets share a corresponding DNA readout probe with Alexa 647 in order to encode a unique color for quantification and identification of this particular mrna species. The complementary readout probe used in this example is 5′-Alex647/ATCCTCCTTCAATACATCCC-3′ (SEQ ID NO:223).

FIG. 7A shows the resulting intensity image of a mouse colon tissue sample with transcripts labeled with Alexa 647 taken on the ISS Alba microscope with FLIM capabilities while FIG. 6C shows the resulting intensity image of a mouse skin tissue sample with transcripts not labeled with anything to serve as a negative control comparison. FIG. 7B shows the phasor plot of the pixels from both images FIGS. 7A and 7C. When gating for the expected lifetime of Alexa 647, only the pixels constituting the labeled UBC mRNA transcripts in FIG. 7A are highlighted. As shown in FIG. 7C versus 7D, upon gating for only the expected lifetime of Alexa 647, only the targets which are labeled in FIG. 7A are correctly detected and identified while the targets which are not labeled in FIG. 7C are correctly not detected. Furthermore, when gated for any other lifetime, only pixels constituting the highly fluctuating autofluorescence background are highlighted. Indicating that background autofluorescence can be identified and separated from the fluorescent signatures emitted by the labeled targets.

### Example 9: Design Lifetime Encoded Probes for Highly Multiplexed Detection Using FRET Dye Pairs

This example further describes an exemplary method as provided herein as depicted in FIG. 4, which illustrates that improved and significant multiplexing capabilities may be achieved by utilizing exemplary methods comprising FRET labeling including modulating the distance between the FRET dye pairs.

In alternative embodiments, probe barcoding methods as provided herein further comprise combinatorial FRET-based labelling where the fluorescent spectrum and/or lifetime resulting from FRET between two adjacent fluorophores is unique to each fluorophore combination (FIG. 2B). In this approach, for example, our codebook can be expanded to 280 target molecules and include at least 16 unique FRET pairs. In another embodiment, FRET decay in proportion to the 6th power of the Forster radius, thus we can use the distance between fluorophores to program different FRET-dependent spectra and/or lifetimes into our molecular probes (FIG. 2C). Uniquely, this molecular programming approach, using nucleic acids to direct FRET behavior, allows sub-5 nm precision to resolve different lifetimes. The distance-FRET labelling approach can expand our codebook to 560 target molecules by separating fluorophores by a specified number of base pairs to achieve, for example, 25%, 50%, 75% and 100% FRET efficiency, depending on the specific donor-acceptor pair Förster radius. Thus, many different barcodes from different targets can be decoded within the same diffraction-limited voxel. Therefore, FRET phenomena can be used as an error correction mechanism at the nanometer level to resolve multiple target molecules in the same voxel.

In alternative embodiments, each group (for example, 10 groups total) may utilize the same fluorophores for the FRET pair but with varying distances between the donor and acceptor. In this particular example, a mouse 3T3 fibroblast sample may contain 10 different gene expression targets with each labeled with the donor, Alexa 594, and the acceptor, BHQ-2. Each gene expression target may have 40 primary probes that bind to its complementary region but with varying length. More specifically, each target may have a set of primary probes that have a binding region for the donor as well as the acceptor but varies the distance between them in order to elicit 10 different FRET signatures. For example, the longest probe, probe 1, may be 160 nt and have a 25 nt gap between the donor and quencher fluorophore. The second longest probe, probe 2, may be 158 nt and have a 23 nt gap between the donor and quencher fluorophore. Each successive probe may be 2 nt shorter than its previous longer counterpart, spanning a distance range between donor and quencher of 7 nt to 25 nt long. To carry out detection, identification, and quantification of this 10 plex panel, the samples may first be fixed with paraformaldehyde and permeabilized with some denaturing or proteolytic agents. The samples may then be blocked and incubated in blocking buffer with the corresponding DNA primary probes. Upon labeling with the same donor and quencher secondary probes, the samples may then be imaged on a microscope with FLIM capabilities with a FOV highlighted as depicted in FIG. 4A. To detect the differential FRET response from the Alexa 594 and BHQ-2 pair, a white laser selected for optimal excitation at 594 nm may be utilized while an AOTF crystal filter may be used to detect optimal emission from the Alexa 594 probe. A1 Airy unit pinhole may be used and frames may be taken until a total 1 million photons were collected from the sample. FIG. 4B illustrates a representative phasor plot of the lifetime positions of each pixel that comprised a taken image. Each pixel in the image may contribute to a position on the phasor plot, where, in this case, 10 different populations can be segmented. Each population can represent a different target with a unique encoding label based on the molecular interactions of distance-based FRET labeling. This barcode labeling scheme permits enormous simultaneous multiplexing capabilities while using only a minimal number of probes. Showing in FIG. 4C is a representative remapped image where each target is analyzed for its lifetime and/or intensity signature for identification. 10 different targets can be identified in this field of view with this approach.

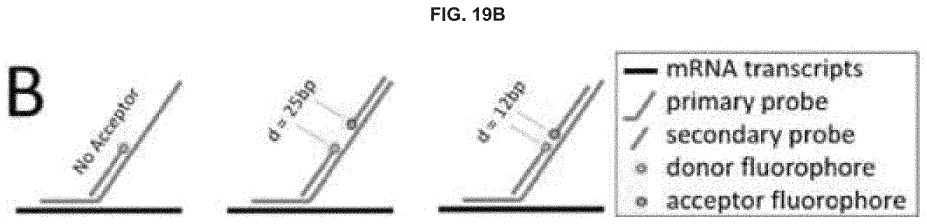

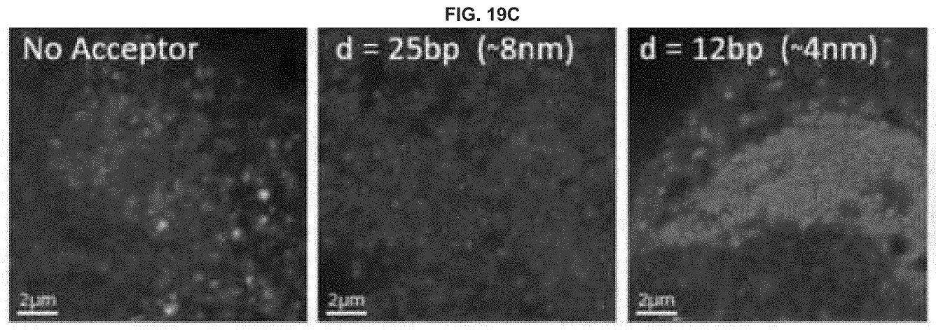

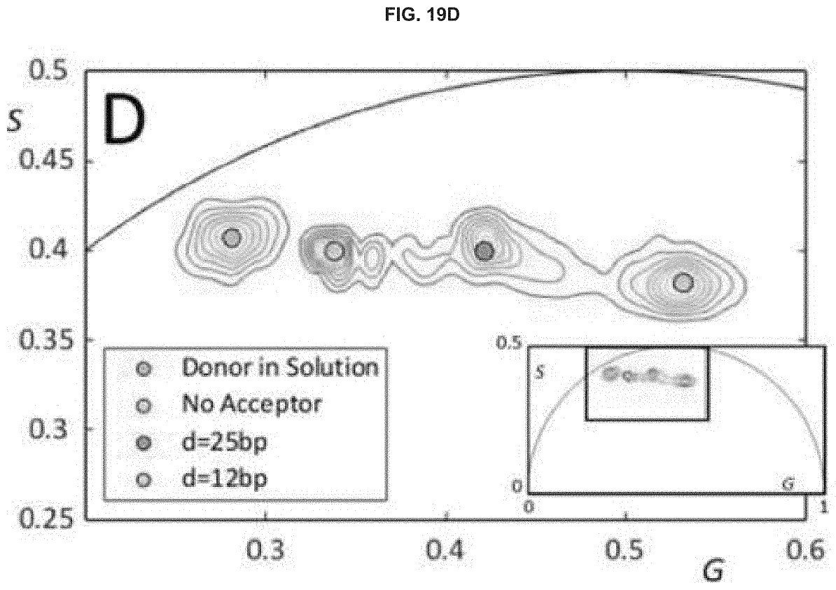

FIG. 19 further illustrates the use of FRET-based approach where different FRET pairs and their distances can be readily modulated to elicit changes in both spectral and lifetime to further enable greater multiplexing.

### Example 10: Immuno-Oncology Marker Panels for Clinical Tumor Biopsy Analyses

Methods and technologies as provided herein can be used to profile immuno-oncology marker panels for cancer tissue biopsy analysis for cancer diagnosis, prognosis and treatment stratification applications. For example, in alternative embodiments, in a clinical melanoma tissue model which represents one of the most established tumor types for immune checkpoint inhibitors, protein targets can be chosen based on their immuno-oncology applications and an exemplary panel can comprise markers for tumor cells (epithelial Pan-cytokeratins, melanoma antigen SOX10), immune cell subsets: T cells (CD3, CD4 and CD8), B cells (CD20), macrophages (CD68), and Tregs (FOXP3), myeloid-derived suppressor cells (CD11b), and immune exhaustion (PD-L1, TIGIT, LAG3). The marker panel can be expanded to cover additional tumor, immune and stromal cell subtypes and checkpoint proteins which are known in the art. For mRNA (co)detection, corresponding mRNAs for protein markers mentioned above, along with melanoma markers (e.g. PMEL) and housekeeping genes (e.g. POLR2A and K10) can be included as examples. Quantification of the number of effector T cell subtypes per region of interest (ROI) in tumor sections and their spatial colocalization with tumor cells can be used to correlate with clinical outcomes for stratified patient care.

### Example 11: In Situ Hybridization Probe Design

To rapidly design in situ hybridization oligo probes for each gene, provided herein is a modified python platform, OligoMiner™, a validated pipeline for rapid design of oligo FISH probes. The primary probes comprise complementary sequence of typically 27-30 nucleotides (nt) and are designed mostly within the coding sequence (CDS), which has fewer variation than the untranslated region (UTR), and optionally within the non-coding sequences (e.g. introns and long RNAs). Furthermore, primary probe “read-out” domains and secondary probes (typically 15 to 20 nt long) can be designed to be orthogonal to each other to avoid off-target binding. Libraries and databases of over 200,000 orthogonal sequences are available in the art and we can simply use those that have been previously validated.

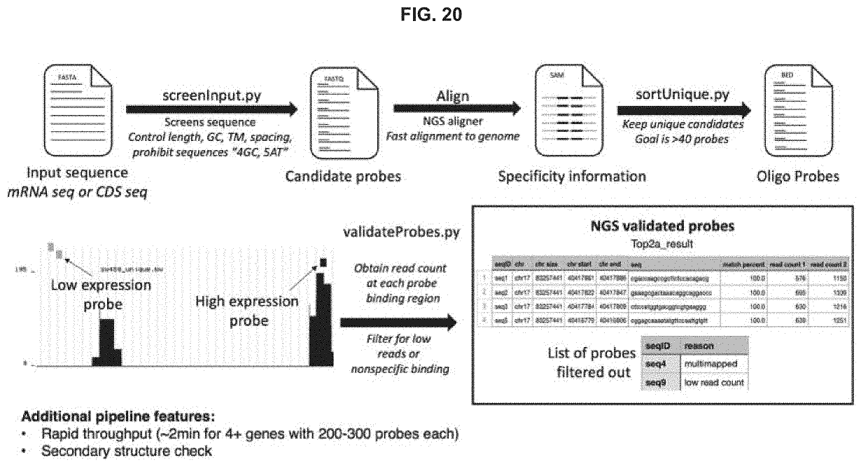

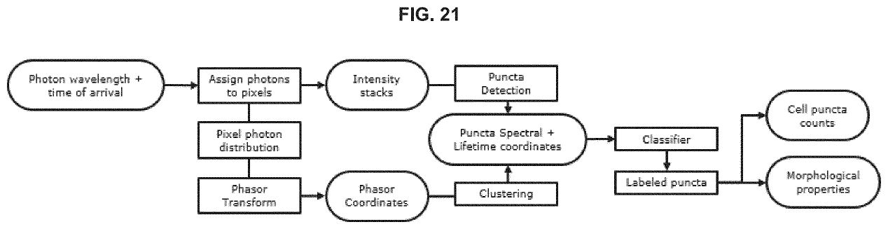

In alternative embodiments, as illustrated in FIG. 20, an exemplary automated high-throughput probe design pipeline uses the mRNA or coding sequence file as the input file to generate a list of probes that bind to the input file sequence while adhering to the user define parameters (length, GC %, melting temperature, spacing, prohibited sequences, etc). In alternative embodiments, non-coding sequences (e.g. introns and long RNAs) can be used as input file sequence for probe designs. The list of probes is then aligned to the genome using an NGS (next generation sequencing) aligner (Bowtie2) to determine if the sequence is unique and specific to the target region. The probes are then tested for uniqueness using BLAT and mapped to the genome and/or transcriptome. Those that appear more than once in the genome are labelled as “multi-mapped” and placed into a list of removed probes. The positions of each unique candidate probes are extracted and used to determine the read count of those regions from next-generation sequencing data. The user defines the threshold numerical value of what is considered as high read count. Probes considered as high read count or high expression, are then placed into a final list while probes with low read counts are placed in the list of removed probes. Multiple next-generation sequencing datasets can be used with the read counts being averaged between the datasets to determine if they are considered a high read count region. Probes binding to high read count regions will have better signal. The final list of probes includes the target name, probe sequence ID, probe sequence, length, percent of sequence aligned, chromosome location, genome position, and read count from each next-generation sequencing dataset. For instance, this automated probe design pipeline can design 200 to 300 probes each for 4 RNA genes within several minutes, greatly reducing the time required for probe design. By obtaining the read count information, probes designed with this method ensure higher success rate of hybridization.

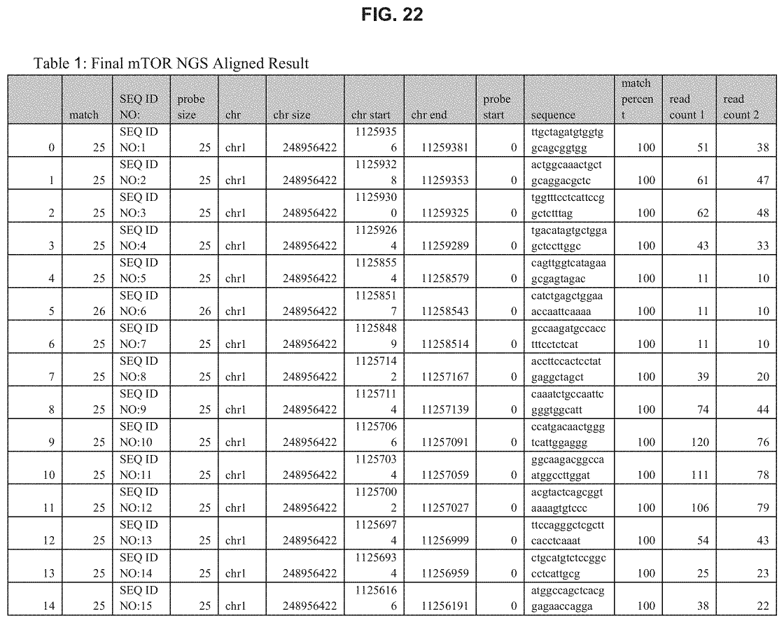

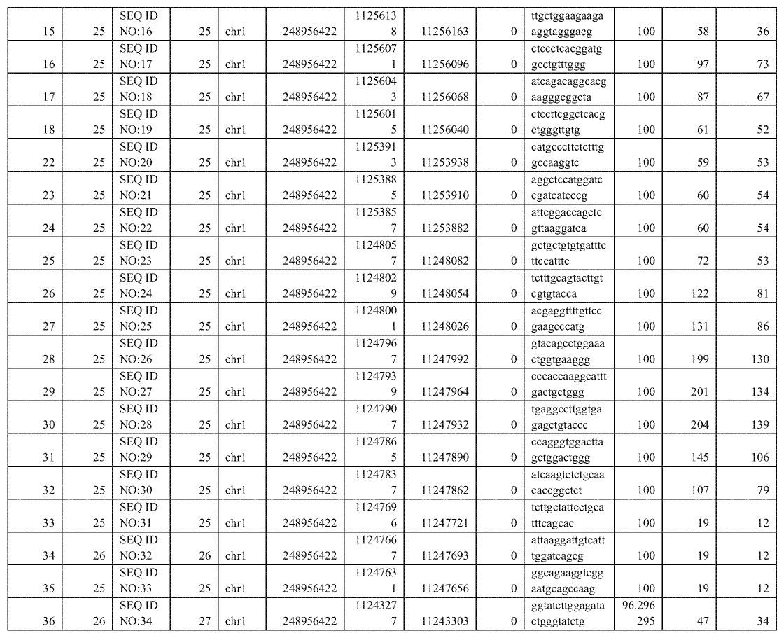

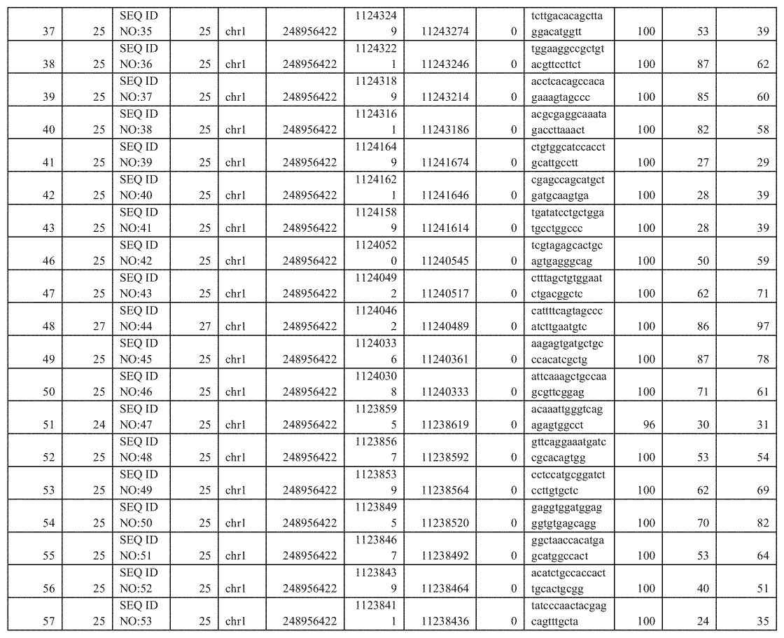

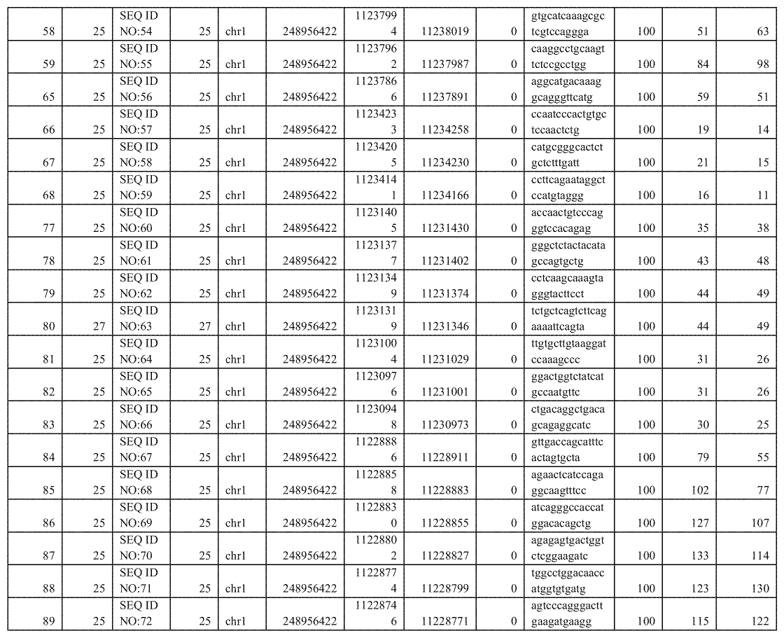

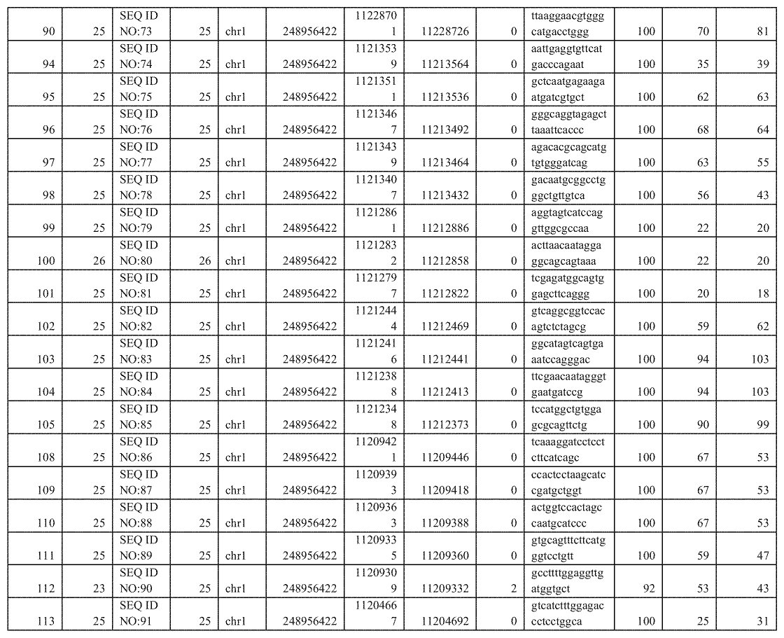

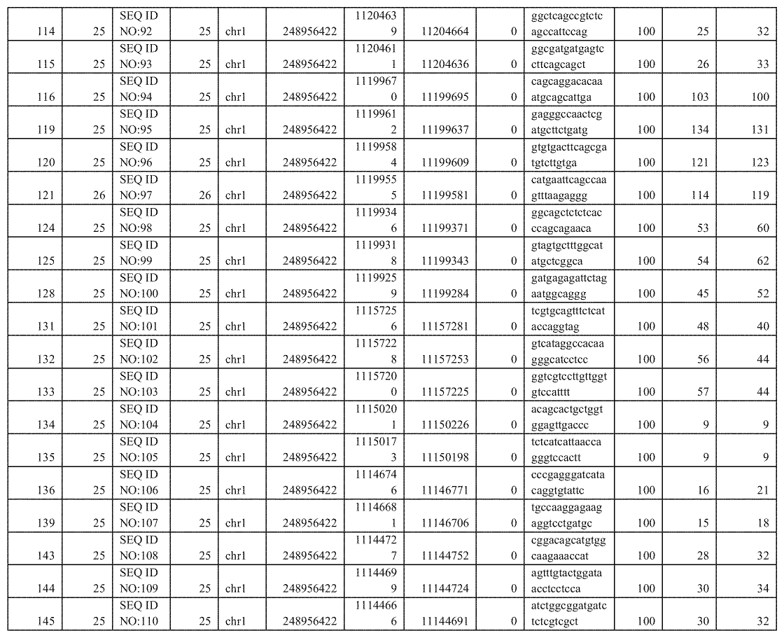

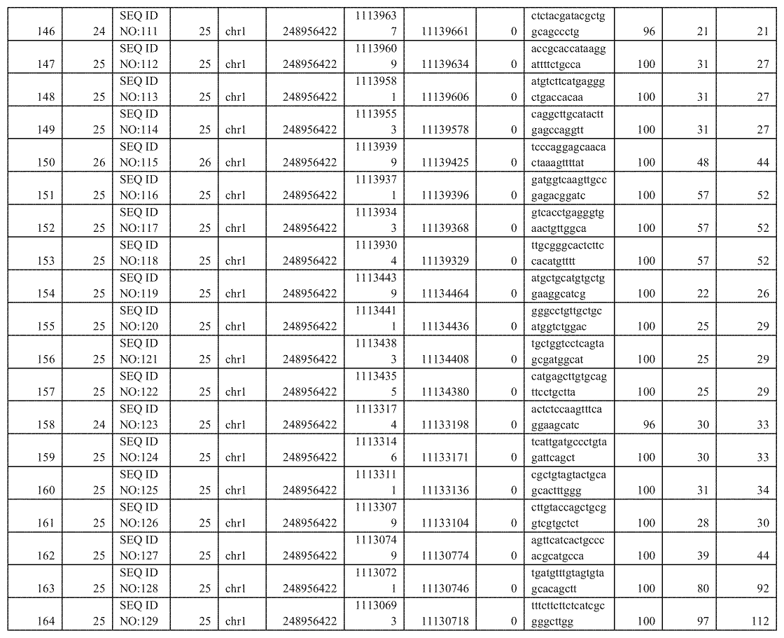

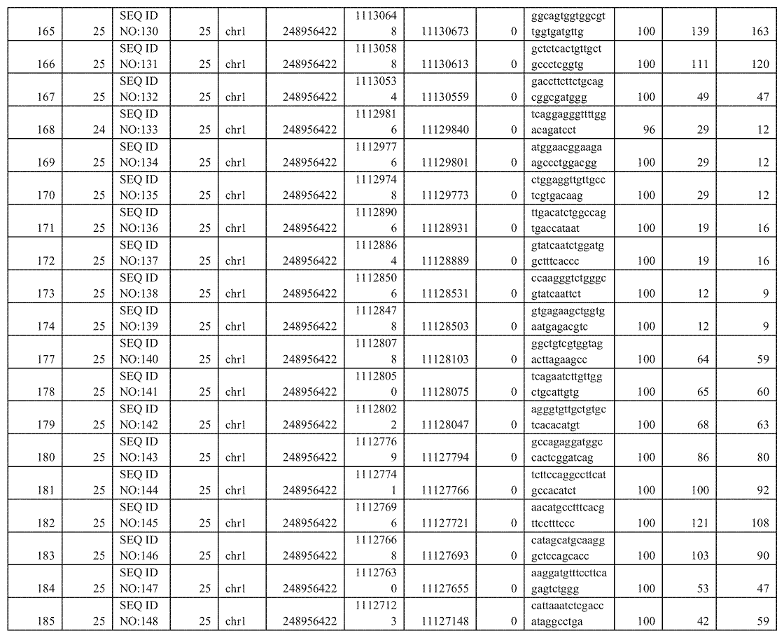

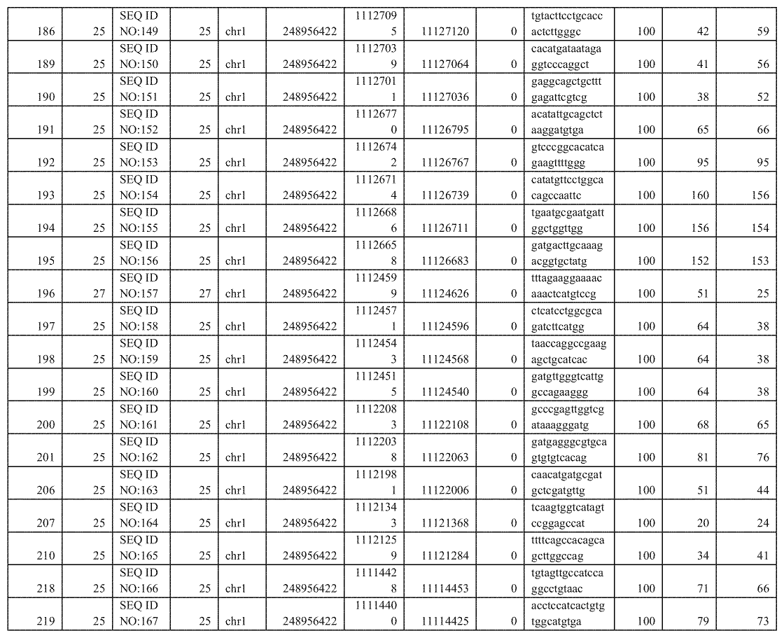

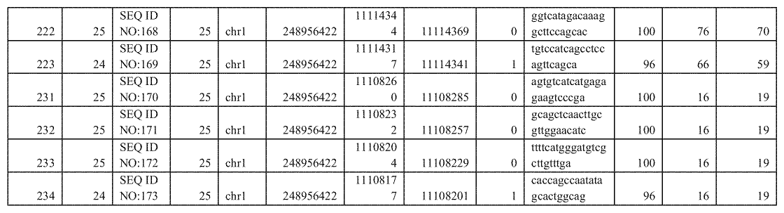

Table 1, illustrated in FIG. 22, shows: mTOR NGS (next generation sequencing) Aligned Result. A table of NGS validated mTOR probes (SEQ ID NO:1 to SEQ ID NO:173) generated by the exemplary BLAT_Aligner script which removes probes that are nonspecific using BLAT and aligns the NGS data with probes for this gene to obtain the read count for each probe region. Each probe includes the following information: number of base pairs that align, sequenceID, probe size, chromosome number, chromosome size, chromosome start position, chromosome end position, probe start, sequence, match percentage, read count average from NGS dataset 1, and read count average from NGS dataset 2 (if available).mTOR

Removed Entries: Table 2 illustrates mTOR removed entries from BLAT_Aligner script. The script filters for probes that BLAT determines as specific, appearing once. Those that appear more than once are labeled as “multimapped” and added to the removed entries list instead of the final list of NGS validated probes. The user defined parameter of “low read count” will determine if the probe is considered to align to a low read count region, based on the average read count of one or more NGS datasets. If the average is below the user defined value, they are removed and added to this list, with that label “low read count”.

TABLE 2

| SEQ ID NO: | reason | sequence |
|---|---|---|
| SEQ ID NO: 174 | multimapped | TCAAATCCCTTCTCTGCTTCTTCAA |
| SEQ ID NO: 175 | multimapped | TTAGTCCCACTGCCAGCATGGGCTC |
| SEQ ID NO: 176 | multimapped | CAAACACCTGGTCATTCAGAGCCAC |
| SEQ ID NO: 177 | multimapped | CTGAGTCGGCCCACAGTGCAGATGG |
| SEQ ID NO: 178 | multimapped | GTGCCAATTCTCCTATTGTTGCCAG |
| SEQ ID NO: 179 | low read count | CATTTCCTCATTTCCAGGCCACTAA |
| SEQ ID NO: 180 | low read count | CTGGAGCATGTCCATGATGATAATAAAA |
| SEQ ID NO: 181 | multimapped | CCTGCCTTTTGGCCAACAAAGAGGA |
| SEQ ID NO: 182 | low read count | AACATTCCCAGCTGCTGGAACAAAA |
| SEQ ID NO: 183 | low read count | TCTGATGTGGCTCTTCACAAAGGAC |
| SEQ ID NO: 184 | low read count | CTCATGAGGGTGACTATTTCATCCATATA |
| SEQ ID NO: 185 | low read count | GGTGTCGCACCAGAACTTTATTCAC |
| SEQ ID NO: 186 | low read count | AGCACATCATAGCGCTGATGATTGA |
| SEQ ID NO: 187 | multimapped | TCATCCTGTTGATCTTCATTCAGTTCA |
| SEQ ID NO: 188 | multimapped | CCATTGTCATCTCTCAGTGGCAGGG |
| SEQ ID NO: 189 | multimapped | CCTTTCTGGAACTCCAGTTCTTTGT |
| SEQ ID NO: 190 | low read count | TCCGGCTGCTGTAGCTTATTATTAAT |
| SEQ ID NO: 191 | low read count | GGCATATTCTAACACTCCGGCCGCT |
| SEQ ID NO: 192 | multimapped | GCCAGCACAGCTCTATAAAATGCCC |
| SEQ ID NO: 193 | low read count | GGTCCCTGGCCTTGTCAATGCACTG |
| SEQ ID NO: 194 | low read count | GCCATCGCAGTTAATTCAGCATCCA |
| SEQ ID NO: 195 | low read count | CCCATATGCCCGACTGTAACTCTCT |
| SEQ ID NO: 196 | multimapped | GTCAGTGGGTAGATGAGGGCCTGGG |
| SEQ ID NO: 197 | multimapped | GTGAGGTCCTTGACATTCCCTGATT |
| SEQ ID NO: 198 | multimapped | AGGATCTTCTTCTTCTCCCTGTAGTC |
| SEQ ID NO: 199 | multimapped | TCAAACACCTCCACCTTCTGCATCA |
| SEQ ID NO: 200 | low read count | CCGCTAAAGAACGGGTATAATTGGTT |
| SEQ ID NO: 201 | low read count | GGCCTAAAATATACCCAACCATTGACA |
| SEQ ID NO: 202 | low read count | CAGGATCTTCCCACTCAGACGGTCC |
| SEQ ID NO: 203 | multimapped | AATGGAATCTTCTCTGGAAACTTCTCT |
| SEQ ID NO: 204 | low read count | CATAGCATTGGTCAACATTCTTGTTAGT |
| SEQ ID NO: 205 | multimapped | CATGACACTGTCCTTGTGCTCTCGC |
| SEQ ID NO: 206 | low read count | TCGGGATCGCTTGTTGCCTTTGGTA |
| SEQ ID NO: 207 | low read count | GGCCAGCAGAGTAGGAATCCGTCCT |
| SEQ ID NO: 208 | low read count | CGTTTTCTTATGGGCTGGCTCTCCA |
| SEQ ID NO: 209 | multimapped | AATGAATAGATTCTGGCACTGTGGT |
| SEQ ID NO: 210 | low read count | TCTGGATAGCTTTCTTATTTAGGGCC |
| SEQ ID NO: 211 | low read count | GTGAGCTTATCTCGAACCCTGTTAA |

A number of embodiments of the invention have been described. Nevertheless, it can be understood that various modifications may be made without departing from the spirit and scope of the invention. Accordingly, other embodiments are within the scope of the following claims.

1. A method for spatially determining, visualizing or quantifying target biological materials, comprising:
(a) providing a biological sample;
(b) in situ staining of the sample with one or a plurality of probes labeled with light-emitting moieties that exhibit or are encoded with a distinct or defined luminescence lifetime characteristics or properties, wherein the one or the plurality of probes specifically bind to the target biological materials,
and optionally the one or a plurality of probes also exhibit or are encoded with a distinct spectrum,
and optionally the distinct or defined luminescence lifetime characteristics or properties of the light-emitting moieties of the plurality of probes comprise or are defined by characteristics, numbers, orders, positions, patterns, configurations, orientations, and interactions modulated by distance, structural and/or architectural relations of the plurality of probes;
(c) imaging of the biological sample using time-resolved luminescence, hyper-spectrally resolved luminescence, or time a hyper-spectrally resolved luminescence; and
(d) measuring the spatial profiles of the target biological materials in the biological sample.

2. The method of claim 1, wherein the biological sample comprises cells, a tissue, a fresh frozen tissue, a formalin-fixed paraffin-embedded (FFPE) tissue, an optimum cutting temperature (OCT) preserved tissue, a biopsy or an organism.

3. The method of claim 2, wherein cells comprise mammalian cells, and optionally the mammalian cells comprise human or mouse cells, or are derived from human or mouse cells.

4. The method of claim 1, wherein:
(a) the target biological materials comprise an RNA, and optionally the RNA comprises an mRNA, and optionally the target biological materials comprise a DNA, and optionally the DNA comprises a chromosomal DNA or a genomic DNA;
(b) the target biological materials comprise a protein or a peptide, and optionally the protein or peptide comprises an epitope;
(c) the target biological materials comprise multiple types of omics markers, wherein optionally the omics markers comprise nucleic acids and proteins, and optionally the omics markers are detected simultaneously;
(d) the one or the plurality of probes comprise an nucleic acid probes or a plurality of nucleic acid probes, or an oligonucleotide or a plurality of pooled oligonucleotides,
and optionally the nucleic acid or oligonucleotide probes have an average length of between about 6 and 300 nucleotides,
and optionally the one or the plurality of probes comprises an antibody-oligonucleotide conjugate, and optionally the one or the plurality of probes comprise a readout domain or domains that allow further binding of a plurality of additional probes,
and optionally the readout domain or domains are generated through a target-binding mediated event, and optionally the target-binding mediated event comprises an enzymatic or a branched amplification event;
(e) the target biological materials comprise a plurality of target molecules, and each target molecule is stained with (or is specifically bound by) 1 probe, at least about 2 probes, at least about 3 probes, at least about 4 probes, at least about 5 probes, at least about 10 probes, at least about 20 probes, at least about 30 probes, at least about 40 probes, at least about 50 probes, at least about 100 probes or more, or wherein each target molecule is stained with or is specifically bound by between about 2 and 100 probes, or between about 5 and 50 probes;
(f) the biological sample is stained with a plurality of same or different probes simultaneously or sequentially, or wherein the in situ staining of the biological sample comprises staining with a plurality of probes simultaneously or sequentially; and/or
(g) the light-emitting moieties comprise fluorophores that exhibit lifetime ranging from between about 0.2 nanoseconds to about 20 nanoseconds.

5.-13. (canceled)

14. The method of claim 1, wherein the time-resolved luminescence comprises a Fluorescence Lifetime Imaging Microscope (FLIM) comprising:
(a) irradiating the stained sample with a modulated light source;
(b) detecting photons emitted by the sample using a detector or a set of detectors;
(c) measuring and analyzing a multitude of emitting species comprising use of a phasor, or a spectral phasor, approach, wherein optionally the analyzing comprises use of spectra-phasor;
(d) analyzing multiple lifetime and spectral components in single pixels using an algorithm; and
(e) identifying and quantitating the target biological molecules at single-molecule resolution from a static or time-lapse 2D image or 3D z-stack, optionally using an image-processing component.

15. The method of claim 14, wherein the multi-component analysis phasor algorithm allows unmixing multiple lifetime and spectral components in the same pixel of an image and is used to ensure fidelity of target detection and to decode a plurality of target moieties within the same diffraction-limited voxel.

16. The method of claim 1, wherein the time-resolved luminescence imaging and analysis are further combined with spectral or hyperspectral imaging comprising parallel Digital Frequency Domain (DFD) electronics or camera-based system light sheet imaging with a multidimensional phasor,
and optionally the hyperspectral imaging and/or lifetime imaging system is equipped with sine/cosine filters.

17. (canceled)

18. The method of claim 1, wherein one, two, three, four, five, six, seven, eight, nine, ten, 100, 1,000, or 10,000 or more different nucleic acid or protein molecules are simultaneously detected or imaged on the same sample in a multiplex fashion, wherein optionally the nucleic acid comprises an RNA or a DNA.

19. The method of claim 1, further comprising placing the biological sample in a compartment that allows fluid flow for processing the sample, and optionally the compartment that allows fluid flow comprises a microfluidic system.

20. A method for designing combinatory, luminescence spectrum and/or lifetime encoded probes and using them to detect target molecules, comprising:
(a) providing a target molecule or a plurality of target molecules in a sample, wherein optionally the sample is a biological sample, and optionally the biological sample comprises a cell, and optionally the cell is a mammalian or a human cell;
(b) providing a plurality of probes that:
(i) specifically bind to the target molecule(s), and
(ii) comprise a label comprising a light-emitting moiety that exhibits a distinct luminescence lifetime characteristic or property, and optionally also comprising a spectrum characteristic;
(c) contacting the plurality of probes with the target molecule or the plurality of target molecules under conditions wherein the plurality of probes can specifically bind to the target molecule or the plurality of target molecules, thereby combinatorially labeling the target molecule or the plurality of target molecules; and
(d) detecting and measuring the specific binding of the plurality of probes with the target molecule or the plurality of target molecules using a time-resolved luminescence method,
wherein when measured and analyzed using the time-resolved luminescence method, each combinatorially labeled target molecule or molecules can elicit a unique luminescence lifetime or property, and optionally also spectrum, signature on a phasor or a spectra-phasor plot, which can identify x, y or x, y, z coordinates of the target molecule or molecules at a single-molecule resolution in the sample,
and optionally further comprising (e), a codebook or index library to decode and identify a target of interest.

21. (canceled)

22. The method of claim 20, wherein:
(a) the luminescence lifetime or property and/or spectrum characteristic comprise or are encoded through, a combinatorial combination of light-emitting moieties' characteristics, numbers, orders, positions, patterns, configurations, orientations, and interactions modulated by distance, structural and architectural relations; and/or
(b) the interactions modulated by distance, structural and architectural relations, or the interactions between light-emitting moieties, are modulated using Forster resonance energy transfer (FRET) comprising use of a FRET pair of dyes,
wherein optionally the distance between the FRET pair of dyes range from 2 nm to 10 nm,
and optionally the FRET phenomena are used as an error correction mechanism at the nanometer level to resolve multiple target molecules in the same voxel.

23. (canceled)

24. A product of manufacture comprising:
(a) a plurality of primary target molecule probes, each primary target molecule probe comprising:
(i) a biorecognition motif with a complementary region which can selectively bind to a specific portion or region of the target molecule in the sample, and
(ii) an extension element or a “read-out” or “adapter” element that can selectively bind to a specific portion or region of a secondary probe;
(b) a second plurality of secondary probes, each secondary probe comprising:
(i) a region which binds specifically to the corresponding extension element on the primary probe, and optionally further comprising a signal amplification or a signal amplification component, and
(ii) a light-emitting moiety or moieties conjugated to one or both ends of the secondary probe with each light-emitting moiety comprising a signal that is distinctly different from each other light-emitting moiety in luminescence spectrum and/or lifetime characteristic.

25. The product of manufacture of claim 24, wherein:
(a) at least one light-emitting moiety comprises a fluorophore;
(b) at least one of the plurality of primary target molecule probes comprises an oligonucleotide; and/or
(c) at least one of the plurality of primary target molecule probes comprises an antibody or antibody binding fragment thereof.

26-38. (canceled)


SEQUENCE LISTING (223 sequences)

SEQ ID NO: 1 - DNA, 25 bp, Artificial Sequence
  Synthetic
ttgctagatgtggtggcagcggtgg

SEQ ID NO: 2 - DNA, 25 bp, Artificial Sequence
  Synthetic
actggcaaactgctgcaggacgctc

SEQ ID NO: 3 - DNA, 25 bp, Artificial Sequence
  Synthetic
tggtttcctcattccggctctttag

SEQ ID NO: 4 - DNA, 25 bp, Artificial Sequence
  Synthetic
tgacatagtgctggagctccttggc

SEQ ID NO: 5 - DNA, 25 bp, Artificial Sequence
  Synthetic
cagttggtcatagaagcgagtagac

SEQ ID NO: 6 - DNA, 26 bp, Artificial Sequence
  Synthetic
catctgagctggaaaccaattcaaaa

SEQ ID NO: 7 - DNA, 25 bp, Artificial Sequence
  Synthetic
gccaagatgccacctttcctctcat

SEQ ID NO: 8 - DNA, 25 bp, Artificial Sequence
  Synthetic
accttccactcctatgaggctagct

SEQ ID NO: 9 - DNA, 25 bp, Artificial Sequence
  Synthetic
caaatctgccaattcgggtggcatt

SEQ ID NO: 10 - DNA, 25 bp, Artificial Sequence
  Synthetic
ccatgacaactgggtcattggaggg

SEQ ID NO: 11 - DNA, 25 bp, Artificial Sequence
  Synthetic
ggcaagacggccaatggccttggat

SEQ ID NO: 12 - DNA, 25 bp, Artificial Sequence
  Synthetic
acgtactcagcggtaaaagtgtccc

SEQ ID NO: 13 - DNA, 25 bp, Artificial Sequence
  Synthetic
ttccagggctcgcttcacctcaaat

SEQ ID NO: 14 - DNA, 25 bp, Artificial Sequence
  Synthetic
ctgcatgtctccggccctcattgcg

SEQ ID NO: 15 - DNA, 25 bp, Artificial Sequence
  Synthetic
atggccagctcacggagaaccagga

SEQ ID NO: 16 - DNA, 25 bp, Artificial Sequence
  Synthetic
ttgctggaagaagaaggtagggacg

SEQ ID NO: 17 - DNA, 25 bp, Artificial Sequence
  Synthetic
ctccctcacggatggcctgtttggg

SEQ ID NO: 18 - DNA, 25 bp, Artificial Sequence
  Synthetic
atcagacaggcacgaagggcggcta

SEQ ID NO: 19 - DNA, 25 bp, Artificial Sequence
  Synthetic
ctccttcggctcacgctgggttgtg

SEQ ID NO: 20 - DNA, 25 bp, Artificial Sequence
  Synthetic
catgcccttctctttggccaaggtc

SEQ ID NO: 21 - DNA, 25 bp, Artificial Sequence
  Synthetic
aggctccatggatccgatcatcccg

SEQ ID NO: 22 - DNA, 25 bp, Artificial Sequence
  Synthetic
attcggaccagctcgttaaggatca

SEQ ID NO: 23 - DNA, 25 bp, Artificial Sequence
  Synthetic
gctgctgtgtgatttcttccatttc

SEQ ID NO: 24 - DNA, 25 bp, Artificial Sequence
  Synthetic
tctttgcagtacttgtcgtgtacca

SEQ ID NO: 25 - DNA, 25 bp, Artificial Sequence
  Synthetic
acgaggttttgttccgaagcccatg

SEQ ID NO: 26 - DNA, 25 bp, Artificial Sequence
  Synthetic
gtacagcctggaaactggtgaaggg

SEQ ID NO: 27 - DNA, 25 bp, Artificial Sequence
  Synthetic
cccaccaaggcatttgactgctggg

SEQ ID NO: 28 - DNA, 25 bp, Artificial Sequence
  Synthetic
tgaggccttggtgagagctgtaccc

SEQ ID NO: 29 - DNA, 25 bp, Artificial Sequence
  Synthetic
ccagggtggacttagctggactggg

SEQ ID NO: 30 - DNA, 25 bp, Artificial Sequence
  Synthetic
atcaagtctctgcaacaccggctct

SEQ ID NO: 31 - DNA, 25 bp, Artificial Sequence
  Synthetic
tcttgctattcctgcatttcagcac

SEQ ID NO: 32 - DNA, 26 bp, Artificial Sequence
  Synthetic
attaaggattgtcatttggatcagcg

SEQ ID NO: 33 - DNA, 25 bp, Artificial Sequence
  Synthetic
ggcagaaggtcggaatgcagccaag

SEQ ID NO: 34 - DNA, 26 bp, Artificial Sequence
  Synthetic
ggtatcttggagatactgggtatctg

SEQ ID NO: 35 - DNA, 25 bp, Artificial Sequence
  Synthetic
tcttgacacagcttaggacatggtt

SEQ ID NO: 36 - DNA, 25 bp, Artificial Sequence
  Synthetic
tggaaggccgctgtacgttccttct

SEQ ID NO: 37 - DNA, 25 bp, Artificial Sequence
  Synthetic
acctcacagccacagaaagtagccc

SEQ ID NO: 38 - DNA, 25 bp, Artificial Sequence
  Synthetic
acgcgaggcaaatagaccttaaact

SEQ ID NO: 39 - DNA, 25 bp, Artificial Sequence
  Synthetic
ctgtggcatccacctgcattgcctt

SEQ ID NO: 40 - DNA, 25 bp, Artificial Sequence
  Synthetic
cgagccagcatgctgatgcaagtga

SEQ ID NO: 41 - DNA, 25 bp, Artificial Sequence
  Synthetic
tgatatcctgctggatgcctggccc

SEQ ID NO: 42 - DNA, 25 bp, Artificial Sequence
  Synthetic
tcgtagagcactgcagtgagggcag

SEQ ID NO: 43 - DNA, 25 bp, Artificial Sequence
  Synthetic
ctttagctgtggaatctgacggctc

SEQ ID NO: 44 - DNA, 27 bp, Artificial Sequence
  Synthetic
cattttcagtagcccatcttgaatgtc

SEQ ID NO: 45 - DNA, 25 bp, Artificial Sequence
  Synthetic
aagagtgatgctgcccacatcgctg

SEQ ID NO: 46 - DNA, 25 bp, Artificial Sequence
  Synthetic
attcaaagctgccaagcgttcggag

SEQ ID NO: 47 - DNA, 24 bp, Artificial Sequence
  Synthetic
acaaattgggtcagagagtggcct

SEQ ID NO: 48 - DNA, 25 bp, Artificial Sequence
  Synthetic
gttcaggaaatgatccgcacagtgg

SEQ ID NO: 49 - DNA, 25 bp, Artificial Sequence
  Synthetic
cctccatgcggatctccttgtgctc

SEQ ID NO: 50 - DNA, 25 bp, Artificial Sequence
  Synthetic
gaggtggatggagggtgtgagcagg

SEQ ID NO: 51 - DNA, 25 bp, Artificial Sequence
  Synthetic
ggctaaccacatgagcatggccact

SEQ ID NO: 52 - DNA, 25 bp, Artificial Sequence
  Synthetic
acatctgccaccacttgcactgcgg

SEQ ID NO: 53 - DNA, 25 bp, Artificial Sequence
  Synthetic
tatcccaactacgagcagtttgcta

SEQ ID NO: 54 - DNA, 25 bp, Artificial Sequence
  Synthetic
gtgcatcaaagcgctcgtccaggga

SEQ ID NO: 55 - DNA, 25 bp, Artificial Sequence
  Synthetic
caaggcctgcaagttctccgcctgg

SEQ ID NO: 56 - DNA, 25 bp, Artificial Sequence
  Synthetic
aggcatgacaaaggcagggttcatg

SEQ ID NO: 57 - DNA, 25 bp, Artificial Sequence
  Synthetic
ccaatcccactgtgctccaactctg

SEQ ID NO: 58 - DNA, 25 bp, Artificial Sequence
  Synthetic
catgcgggcactctgctctttgatt

SEQ ID NO: 59 - DNA, 25 bp, Artificial Sequence
  Synthetic
ccttcagaataggctccatgtaggg

SEQ ID NO: 60 - DNA, 25 bp, Artificial Sequence
  Synthetic
accaactgtcccagggtccacagag

SEQ ID NO: 61 - DNA, 25 bp, Artificial Sequence
  Synthetic
gggctctactacatagccagtgctg

SEQ ID NO: 62 - DNA, 25 bp, Artificial Sequence
  Synthetic
cctcaagcaaagtagggtacttcct

SEQ ID NO: 63 - DNA, 27 bp, Artificial Sequence
  Synthetic
tctgctcagtcttcagaaaattcagta

SEQ ID NO: 64 - DNA, 25 bp, Artificial Sequence
  Synthetic
ttgtgcttgtaaggatccaaagccc

SEQ ID NO: 65 - DNA, 25 bp, Artificial Sequence
  Synthetic
ggactggtctatcatgccaatgttc

SEQ ID NO: 66 - DNA, 25 bp, Artificial Sequence
  Synthetic
ctgacaggctgacagcagaggcatc

SEQ ID NO: 67 - DNA, 25 bp, Artificial Sequence
  Synthetic
gttgaccagcatttcactagtgcta

SEQ ID NO: 68 - DNA, 25 bp, Artificial Sequence
  Synthetic
agaactcatccagaggcaagtttcc

SEQ ID NO: 69 - DNA, 25 bp, Artificial Sequence
  Synthetic
atcagggccaccatggacacagctg

SEQ ID NO: 70 - DNA, 25 bp, Artificial Sequence
  Synthetic
agagagtgactggtctcggaagatc

SEQ ID NO: 71 - DNA, 25 bp, Artificial Sequence
  Synthetic
tggcctggacaaccatggtgtgatg

SEQ ID NO: 72 - DNA, 25 bp, Artificial Sequence
  Synthetic
agtcccagggacttgaagatgaagg

SEQ ID NO: 73 - DNA, 25 bp, Artificial Sequence
  Synthetic
ttaaggaacgtgggcatgacctggg

SEQ ID NO: 74 - DNA, 25 bp, Artificial Sequence
  Synthetic
aattgaggtgttcatgacccagaat

SEQ ID NO: 75 - DNA, 25 bp, Artificial Sequence
  Synthetic
gctcaatgagaagaatgatcgtgct

SEQ ID NO: 76 - DNA, 25 bp, Artificial Sequence
  Synthetic
gggcaggtagagcttaaattcaccc

SEQ ID NO: 77 - DNA, 25 bp, Artificial Sequence
  Synthetic
agacacgcagcatgtgtgggatcag

SEQ ID NO: 78 - DNA, 25 bp, Artificial Sequence
  Synthetic
gacaatgcggcctgggctgttgtca

SEQ ID NO: 79 - DNA, 25 bp, Artificial Sequence
  Synthetic
aggtagtcatccaggttggcgccaa

SEQ ID NO: 80 - DNA, 26 bp, Artificial Sequence
  Synthetic
acttaacaataggaggcagcagtaaa

SEQ ID NO: 81 - DNA, 25 bp, Artificial Sequence
  Synthetic
tcgagatggcagtggagcttcaggg

SEQ ID NO: 82 - DNA, 25 bp, Artificial Sequence
  Synthetic
gtcaggcggtccacagtctctagcg

SEQ ID NO: 83 - DNA, 25 bp, Artificial Sequence
  Synthetic
ggcatagtcagtgaaatccagggac

SEQ ID NO: 84 - DNA, 25 bp, Artificial Sequence
  Synthetic
ttcgaacaatagggtgaatgatccg

SEQ ID NO: 85 - DNA, 25 bp, Artificial Sequence
  Synthetic
tccatggctgtggagcgcagttctg

SEQ ID NO: 86 - DNA, 25 bp, Artificial Sequence
  Synthetic
tcaaaggatcctcctcttcatcagc

SEQ ID NO: 87 - DNA, 25 bp, Artificial Sequence
  Synthetic
ccactcctaagcatccgatgctggt

SEQ ID NO: 88 - DNA, 25 bp, Artificial Sequence
  Synthetic
actggtccactagccaatgcatccc

SEQ ID NO: 89 - DNA, 25 bp, Artificial Sequence
  Synthetic
gtgcagtttcttcatgggtcctgtt

SEQ ID NO: 90 - DNA, 23 bp, Artificial Sequence
  Synthetic
gccttttggaggttgatggtgct

SEQ ID NO: 91 - DNA, 25 bp, Artificial Sequence
  Synthetic
gtcatctttggagaccctcctggca

SEQ ID NO: 92 - DNA, 25 bp, Artificial Sequence
  Synthetic
ggctcagccgtctcagccattccag

SEQ ID NO: 93 - DNA, 25 bp, Artificial Sequence
  Synthetic
ggcgatgatgagtccttcagcagct

SEQ ID NO: 94 - DNA, 25 bp, Artificial Sequence
  Synthetic
cagcaggacacaaatgcagcattga

SEQ ID NO: 95 - DNA, 25 bp, Artificial Sequence
  Synthetic
gagggccaactcgatgcttctgatg

SEQ ID NO: 96 - DNA, 25 bp, Artificial Sequence
  Synthetic
gtgtgacttcagcgatgtcttgtga

SEQ ID NO: 97 - DNA, 26 bp, Artificial Sequence
  Synthetic
catgaattcagccaagtttaagaggg

SEQ ID NO: 98 - DNA, 25 bp, Artificial Sequence
  Synthetic
ggcagctctctcacccagcagaaca

SEQ ID NO: 99 - DNA, 25 bp, Artificial Sequence
  Synthetic
gtagtgctttggcatatgctcggca

SEQ ID NO: 100 - DNA, 25 bp, Artificial Sequence
  Synthetic
gatgagagattctagaatggcaggg

SEQ ID NO: 101 - DNA, 25 bp, Artificial Sequence
  Synthetic
tcgtgcagtttctcataccaggtag

SEQ ID NO: 102 - DNA, 25 bp, Artificial Sequence
  Synthetic
gtcataggccacaagggcatcctcc

SEQ ID NO: 103 - DNA, 25 bp, Artificial Sequence
  Synthetic
ggtcgtccttgttggtgtccatttt

SEQ ID NO: 104 - DNA, 25 bp, Artificial Sequence
  Synthetic
acagcactgctggtggagttgaccc

SEQ ID NO: 105 - DNA, 25 bp, Artificial Sequence
  Synthetic
tctcatcattaaccagggtccactt

SEQ ID NO: 106 - DNA, 25 bp, Artificial Sequence
  Synthetic
cccgagggatcatacaggtgtattc

SEQ ID NO: 107 - DNA, 25 bp, Artificial Sequence
  Synthetic
tgccaaggagaagaggtcctgatgc

SEQ ID NO: 108 - DNA, 25 bp, Artificial Sequence
  Synthetic
cggacagcatgtggcaagaaaccat

SEQ ID NO: 109 - DNA, 25 bp, Artificial Sequence
  Synthetic
agtttgtactggataacctcctcca

SEQ ID NO: 110 - DNA, 25 bp, Artificial Sequence
  Synthetic
atctggcggatgatctctcgtcgct

SEQ ID NO: 111 - DNA, 24 bp, Artificial Sequence
  Synthetic
ctctacgatacgctggcagccctg

SEQ ID NO: 112 - DNA, 25 bp, Artificial Sequence
  Synthetic
accgcaccataaggattttctgcca

SEQ ID NO: 113 - DNA, 25 bp, Artificial Sequence
  Synthetic
atgtcttcatgagggctgaccacaa

SEQ ID NO: 114 - DNA, 25 bp, Artificial Sequence
  Synthetic
caggcttgcatacttgagccaggtt

SEQ ID NO: 115 - DNA, 26 bp, Artificial Sequence
  Synthetic
tcccaggagcaacactaaagttttat

SEQ ID NO: 116 - DNA, 25 bp, Artificial Sequence
  Synthetic
gatggtcaagttgccgagacggatc

SEQ ID NO: 117 - DNA, 25 bp, Artificial Sequence
  Synthetic
gtcacctgagggtgaactgttggca

SEQ ID NO: 118 - DNA, 25 bp, Artificial Sequence
  Synthetic
ttgcgggcactcttccacatgtttt

SEQ ID NO: 119 - DNA, 25 bp, Artificial Sequence
  Synthetic
atgctgcatgtgctggaaggcatcg

SEQ ID NO: 120 - DNA, 25 bp, Artificial Sequence
  Synthetic
gggcctgttgctgcatggtctggac

SEQ ID NO: 121 - DNA, 25 bp, Artificial Sequence
  Synthetic
tgctggtcctcagtagcgatggcat

SEQ ID NO: 122 - DNA, 25 bp, Artificial Sequence
  Synthetic
catgagcttgtgcagttcctgctta

SEQ ID NO: 123 - DNA, 24 bp, Artificial Sequence
  Synthetic
actctccaagtttcaggaagcatc

SEQ ID NO: 124 - DNA, 25 bp, Artificial Sequence
  Synthetic
tcattgatgccctgtagattcagct

SEQ ID NO: 125 - DNA, 25 bp, Artificial Sequence
  Synthetic
cgctgtagtactgcagcactttggg

SEQ ID NO: 126 - DNA, 25 bp, Artificial Sequence
  Synthetic
cttgtaccagctgcggtcgtgctct

SEQ ID NO: 127 - DNA, 25 bp, Artificial Sequence
  Synthetic
agttcatcactgcccacgcatgcca

SEQ ID NO: 128 - DNA, 25 bp, Artificial Sequence
  Synthetic
tgatgtttgtagtgtagcacagctt

SEQ ID NO: 129 - DNA, 25 bp, Artificial Sequence
  Synthetic
tttcttcttctcatcgcgggcttgg

SEQ ID NO: 130 - DNA, 25 bp, Artificial Sequence
  Synthetic
ggcagtggtggcgttggtgatgttg

SEQ ID NO: 131 - DNA, 25 bp, Artificial Sequence
  Synthetic
gctctcactgttgctgccctcggtg

SEQ ID NO: 132 - DNA, 25 bp, Artificial Sequence
  Synthetic
gaccttcttctgcagcggcgatggg

SEQ ID NO: 133 - DNA, 24 bp, Artificial Sequence
  Synthetic
tcaggagggttttggacagatcct

SEQ ID NO: 134 - DNA, 25 bp, Artificial Sequence
  Synthetic
atggaacggaagaagccctggacgg

SEQ ID NO: 135 - DNA, 25 bp, Artificial Sequence
  Synthetic
ctggaggttgttgcctcgtgacaag

SEQ ID NO: 136 - DNA, 25 bp, Artificial Sequence
  Synthetic
ttgacatctggccagtgaccataat

SEQ ID NO: 137 - DNA, 25 bp, Artificial Sequence
  Synthetic
gtatcaatctggatggctttcaccc

SEQ ID NO: 138 - DNA, 25 bp, Artificial Sequence
  Synthetic
ccaagggtctgggcgtatcaattct

SEQ ID NO: 139 - DNA, 25 bp, Artificial Sequence
  Synthetic
gtgagaagctggtgaatgagacgtc

SEQ ID NO: 140 - DNA, 25 bp, Artificial Sequence
  Synthetic
ggctgtcgtggtagacttagaagcc

SEQ ID NO: 141 - DNA, 25 bp, Artificial Sequence
  Synthetic
tcagaatcttgttggctgcattgtg

SEQ ID NO: 142 - DNA, 25 bp, Artificial Sequence
  Synthetic
agggtgttgctgtgctcacacatgt

SEQ ID NO: 143 - DNA, 25 bp, Artificial Sequence
  Synthetic
gccagaggatggccactcggatcag

SEQ ID NO: 144 - DNA, 25 bp, Artificial Sequence
  Synthetic
tcttccaggccttcatgccacatct

SEQ ID NO: 145 - DNA, 25 bp, Artificial Sequence
  Synthetic
aacatgcctttcacgttcctttccc

SEQ ID NO: 146 - DNA, 25 bp, Artificial Sequence
  Synthetic
catagcatgcaagggctccagcacc

SEQ ID NO: 147 - DNA, 25 bp, Artificial Sequence
  Synthetic
aaggatgtttccttcagagtctggg

SEQ ID NO: 148 - DNA, 25 bp, Artificial Sequence
  Synthetic
cattaaatctcgaccataggcctga

SEQ ID NO: 149 - DNA, 25 bp, Artificial Sequence
  Synthetic
tgtacttcctgcaccactcttgggc

SEQ ID NO: 150 - DNA, 25 bp, Artificial Sequence
  Synthetic
cacatgataatagaggtcccaggct

SEQ ID NO: 151 - DNA, 25 bp, Artificial Sequence
  Synthetic
gaggcagctgctttgagattcgtcg

SEQ ID NO: 152 - DNA, 25 bp, Artificial Sequence
  Synthetic
acatattgcagctctaaggatgtga

SEQ ID NO: 153 - DNA, 25 bp, Artificial Sequence
  Synthetic
gtcccggcacatcagaagttttggg

SEQ ID NO: 154 - DNA, 25 bp, Artificial Sequence
  Synthetic
catatgttcctggcacagccaattc

SEQ ID NO: 155 - DNA, 25 bp, Artificial Sequence
  Synthetic
tgaatgcgaatgattggctggttgg

SEQ ID NO: 156 - DNA, 25 bp, Artificial Sequence
  Synthetic
gatgacttgcaaagacggtgctatg

SEQ ID NO: 157 - DNA, 27 bp, Artificial Sequence
  Synthetic
tttagaaggaaaacaaactcatgtccg

SEQ ID NO: 158 - DNA, 25 bp, Artificial Sequence
  Synthetic
ctcatcctggcgcagatcttcatgg

SEQ ID NO: 159 - DNA, 25 bp, Artificial Sequence
  Synthetic
taaccaggccgaagagctgcatcac

SEQ ID NO: 160 - DNA, 25 bp, Artificial Sequence
  Synthetic
gatgttgggtcattggccagaaggg

SEQ ID NO: 161 - DNA, 25 bp, Artificial Sequence
  Synthetic
gcccgagttggtcgataaagggatg

SEQ ID NO: 162 - DNA, 25 bp, Artificial Sequence
  Synthetic
gatgagggcgtgcagtgtgtcacag

SEQ ID NO: 163 - DNA, 25 bp, Artificial Sequence
  Synthetic
caacatgatgcgatgctcgatgttg

SEQ ID NO: 164 - DNA, 25 bp, Artificial Sequence
  Synthetic
tcaagtggtcatagtccggagccat

SEQ ID NO: 165 - DNA, 25 bp, Artificial Sequence
  Synthetic
ttttcagccacagcagcttggccag

SEQ ID NO: 166 - DNA, 25 bp, Artificial Sequence
  Synthetic
tgtagttgccatccaggcctgtaac

SEQ ID NO: 167 - DNA, 25 bp, Artificial Sequence
  Synthetic
acctccatcactgtgtggcatgtga

SEQ ID NO: 168 - DNA, 25 bp, Artificial Sequence
  Synthetic
ggtcatagacaaaggcttccagcac

SEQ ID NO: 169 - DNA, 24 bp, Artificial Sequence
  Synthetic
tgtccatcagcctccagttcagca

SEQ ID NO: 170 - DNA, 25 bp, Artificial Sequence
  Synthetic
agtgtcatcatgagagaagtcccga

SEQ ID NO: 171 - DNA, 25 bp, Artificial Sequence
  Synthetic
gcagctcaacttgcgttggaacatc

SEQ ID NO: 172 - DNA, 25 bp, Artificial Sequence
  Synthetic
ttttcatgggatgtcgcttgtttga

SEQ ID NO: 173 - DNA, 24 bp, Artificial Sequence
  Synthetic
caccagccaatatagcactggcag

SEQ ID NO: 174 - DNA, 25 bp, Artificial Sequence
  Synthetic
tcaaatcccttctctgcttcttcaa

SEQ ID NO: 175 - DNA, 25 bp, Artificial Sequence
  Synthetic
ttagtcccactgccagcatgggctc

SEQ ID NO: 176 - DNA, 25 bp, Artificial Sequence
  Synthetic
caaacacctggtcattcagagccac

SEQ ID NO: 177 - DNA, 25 bp, Artificial Sequence
  Synthetic
ctgagtcggcccacagtgcagatgg

SEQ ID NO: 178 - DNA, 25 bp, Artificial Sequence
  Synthetic
gtgccaattctcctattgttgccag

SEQ ID NO: 179 - DNA, 25 bp, Artificial Sequence
  Synthetic
catttcctcatttccaggccactaa

SEQ ID NO: 180 - DNA, 28 bp, Artificial Sequence
  Synthetic
ctggagcatgtccatgatgataataaaa

SEQ ID NO: 181 - DNA, 25 bp, Artificial Sequence
  Synthetic
cctgccttttggccaacaaagagga

SEQ ID NO: 182 - DNA, 25 bp, Artificial Sequence
  Synthetic
aacattcccagctgctggaacaaaa

SEQ ID NO: 183 - DNA, 25 bp, Artificial Sequence
  Synthetic
tctgatgtggctcttcacaaaggac

SEQ ID NO: 184 - DNA, 29 bp, Artificial Sequence
  Synthetic
ctcatgagggtgactatttcatccatata

SEQ ID NO: 185 - DNA, 25 bp, Artificial Sequence
  Synthetic
ggtgtcgcaccagaactttattcac

SEQ ID NO: 186 - DNA, 25 bp, Artificial Sequence
  Synthetic
agcacatcatagcgctgatgattga

SEQ ID NO: 187 - DNA, 27 bp, Artificial Sequence
  Synthetic
tcatcctgttgatcttcattcagttca

SEQ ID NO: 188 - DNA, 25 bp, Artificial Sequence
  Synthetic
ccattgtcatctctcagtggcaggg

SEQ ID NO: 189 - DNA, 25 bp, Artificial Sequence
  Synthetic
cctttctggaactccagttctttgt

SEQ ID NO: 190 - DNA, 26 bp, Artificial Sequence
  Synthetic
tccggctgctgtagcttattattaat

SEQ ID NO: 191 - DNA, 25 bp, Artificial Sequence
  Synthetic
ggcatattctaacactccggccgct

SEQ ID NO: 192 - DNA, 25 bp, Artificial Sequence
  Synthetic
gccagcacagctctataaaatgccc

SEQ ID NO: 193 - DNA, 25 bp, Artificial Sequence
  Synthetic
ggtccctggccttgtcaatgcactg

SEQ ID NO: 194 - DNA, 25 bp, Artificial Sequence
  Synthetic
gccatcgcagttaattcagcatcca

SEQ ID NO: 195 - DNA, 25 bp, Artificial Sequence
  Synthetic
cccatatgcccgactgtaactctct

SEQ ID NO: 196 - DNA, 25 bp, Artificial Sequence
  Synthetic
gtcagtgggtagatgagggcctggg

SEQ ID NO: 197 - DNA, 25 bp, Artificial Sequence
  Synthetic
gtgaggtccttgacattccctgatt

SEQ ID NO: 198 - DNA, 26 bp, Artificial Sequence
  Synthetic
aggatcttcttcttctccctgtagtc

SEQ ID NO: 199 - DNA, 25 bp, Artificial Sequence
  Synthetic
tcaaacacctccaccttctgcatca

SEQ ID NO: 200 - DNA, 26 bp, Artificial Sequence
  Synthetic
ccgctaaagaacgggtataattggtt

SEQ ID NO: 201 - DNA, 27 bp, Artificial Sequence
  Synthetic
ggcctaaaatatacccaaccattgaca

SEQ ID NO: 202 - DNA, 25 bp, Artificial Sequence
  Synthetic
caggatcttcccactcagacggtcc

SEQ ID NO: 203 - DNA, 27 bp, Artificial Sequence
  Synthetic
aatggaatcttctctggaaacttctct

SEQ ID NO: 204 - DNA, 28 bp, Artificial Sequence
  Synthetic
catagcattggtcaacattcttgttagt

SEQ ID NO: 205 - DNA, 25 bp, Artificial Sequence
  Synthetic
catgacactgtccttgtgctctcgc

SEQ ID NO: 206 - DNA, 25 bp, Artificial Sequence
  Synthetic
tcgggatcgcttgttgcctttggta

SEQ ID NO: 207 - DNA, 25 bp, Artificial Sequence
  Synthetic
ggccagcagagtaggaatccgtcct

SEQ ID NO: 208 - DNA, 25 bp, Artificial Sequence
  Synthetic
cgttttcttatgggctggctctcca

SEQ ID NO: 209 - DNA, 25 bp, Artificial Sequence
  Synthetic
aatgaatagattctggcactgtggt

SEQ ID NO: 210 - DNA, 26 bp, Artificial Sequence
  Synthetic
tctggatagctttcttatttagggcc

SEQ ID NO: 211 - DNA, 25 bp, Artificial Sequence
  Synthetic
gtgagcttatctcgaaccctgttaa

SEQ ID NO: 212 - DNA, 25 bp, Artificial Sequence
  Synthetic
cgaccaagccgcttctccacagacg

SEQ ID NO: 213 - DNA, 25 bp, Artificial Sequence
  Synthetic
gaaagcgactaaacaggcaggaccc

SEQ ID NO: 214 - DNA, 25 bp, Artificial Sequence
  Synthetic
cttccatggtgacggtcgtgaaggg

SEQ ID NO: 215 - DNA, 27 bp, Artificial Sequence
  Synthetic
cggagcaaaatatgttccaattgtgtt

SEQ ID NO: 216 - DNA, 50 bp, Artificial Sequence
  Synthetic
ctcgacctttctcttcttcttggggcttttagagtgagtagtagtggagt

SEQ ID NO: 217 - DNA, 27 bp, Artificial Sequence
  Synthetic
ctcgacctttctcttcttcttggggct

SEQ ID NO: 218 - DNA, 20 bp, Artificial Sequence
  Synthetic
agagtgagtagtagtggagt

SEQ ID NO: 219 - DNA, 20 bp, Artificial Sequence
  Synthetic
  modified_base (1)..(1): Alex647
actccactactactcactct

SEQ ID NO: 220 - DNA, 50 bp, Artificial Sequence
  Synthetic
gaggcgaaggaccaggtgcagggtggatttgggatgtattgaaggaggat

SEQ ID NO: 221 - DNA, 27 bp, Artificial Sequence
  Synthetic
gaggcgaaggaccaggtgcagggtgga

SEQ ID NO: 222 - DNA, 20 bp, Artificial Sequence
  Synthetic
gggatgtattgaaggaggat

SEQ ID NO: 223 - DNA, 20 bp, Artificial Sequence
  Synthetic
  modified_base (1)..(1): Alex647
atcctccttcaatacatccc